(19) United States
(12) Patent Application Publication
(10) Pub. No.: US 2023/0303544 A1
(43) Pub. Date: Sep. 28, 2023

(54) LMO2 PROTEIN INHIBITORS

(71) Applicant: UNIVERSITY OF OXFORD, Oxfordshire (GB)

(72) Inventors: Terence Rabbits, Sutton (GB); Nicolas Bery, Oxfordshire (GB); Carole Bataille, Oxfordshire (GB); Angela Russell, Oxfordshire (GB)

(21) Appl. No.: 18/021,508

(22) PCT Filed: Aug. 18, 2021
(86) PCT No.: PCT/GB2021/052143

§ 371 Date: Feb. 15, 2023

(30) Foreign Application Priority Data
Aug. 19, 2020 (GB) 2012969.8

(51) Int. Cl. C07D 413/04; C07D 413/14; C07D 277/42; C07D 263/48; C07D 417/12; C07D 403/14; C07D 403/12; C07D 495/04

(52) U.S. Cl. CPC C07D 413/04; C07D 413/14; C07D 277/42; C07D 263/48; C07D 417/12; C07D 403/14; C07D 403/12; C07D 495/04

(57) ABSTRACT

The present invention relates to compounds of Formula (I) that function as LMO2 activity: Formula (I) wherein R₁, X₁, X₂, X₃, Q, R₂, R₃ and R₄ are each as defined herein. The present invention also relates to processes for the preparation of these compounds, to pharmaceutical compositions comprising them, and to their use in the treatment of proliferative disorders, such as cancer, as well as other diseases or conditions in which LMO2 activity is implicated.

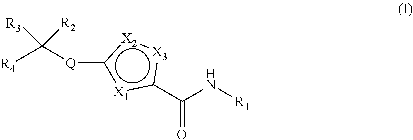

### INTRODUCTION

The present invention relates to certain pharmacologically active compounds that modulate activity of the T cell leukaemia chromosal translocation protein, LIM domain only protein 2 (LMO2). The compounds of the present invention may be used to treat diseases or conditions mediated, at least in part, by inappropriate LMO2 activity, for example hyperproliferative diseases, including cancer. The invention furthermore relates to processes for the preparation of these compounds, their use as pharmaceuticals, and pharmaceutical compositions comprising them.

### BACKGROUND OF THE INVENTION

Tumour-associated aberrant chromosomes that activate oncogenic proteins by gene activation or gene fusion are known to be caused by chromosomal translocations, wherein an unusual rearrangement of chromosomes occurs. Recurring chromosomal translocations are abundant in leukaemias/lymphomas, in sarcomas and in carcinomas (Rabbitts, 2009) and represent a class of tumour-specific proteins that could be targets for therapy. Generally, the products of chromosomal translocations are intracellular proteins and do not have enzyme active sites per se but rather are proteins that function in various cellular processes, such as transcription where protein-protein interactions (PPIs) are critical. PPIs have relatively large interaction surfaces involving several binding hotspots but also usually lack a well-defined binding site (or pocket) (Scott et al., 2016). PPIs, however, can be inhibited by macromolecules such as intracellular antibody fragments (e.g. single chain Fragment variable (scFv) (Cochet et al., 1998; Tanaka and Rabbitts, 2003; Visintin et al., 1999) or intracellular domains antibodies (iDAbs) (Tanaka and Rabbitts, 2010; Tanaka et al., 2011; Tanaka et al., 2007)) and other intracellular antibody-like formats (Bery et al., 2019; Spencer-Smith et al., 2017). The advantages of intracellular antibody-based reagents are that the natural properties of antibodies such as their high affinity and specificity can be exploited. Furthermore, their relatively quick selection processes with methods such as intracellular antibody capture (Visintin et al., 1999) allow their use to investigate their effects on a target disease in relevant preclinical models (target validation) (Tanaka and Rabbitts, 2010; Tanaka et al., 2011; Tanaka et al., 2007).

While the aim of using intracellular antibodies as drugs in their own right (termed macrodrugs (Tanaka and Rabbitts, 2008)) is still being developed, the small size of the iDAb interaction surface with target antigens has been explored as a template for small molecule surrogates in a method called Abd technology (Antibody-derived compound technology) (Quevedo et al., 2018). As reported by Quevedo et al., initial Abd selection was carried out as a biochemical assay in which a competitive surface plasmon resonance (cSPR) method was used where the location of interaction of compounds from a fragment library with HRASG12V was assessed by competition with HRAS-intracellular antibody dimer. Such an in vitro selection method yielded RAS-binding fragment hits that were developed by medicinal chemistry to nM interacting compounds. The in vitro Abd depends on favourable binding properties of the intracellular antibody with its target (very high affinity, high on-rate constant (Kₒₙ) and low off-rate constant (K_{off})) and on the selected compounds having advantageous properties in cellular uptake.

One intracellular protein produced from a chromosomal translocation is LIM domain only protein 2 (LMO2) which is activated by chromosomal translocations t(11;14)(p13;q11) and t(7;11)(q35;p13) in T cell acute lymphoblastic leukaemia (T-ALL) (Chambers and Rabbitts, 2015). LMO2 is overexpressed in more than 50% T-ALL (Ferrando and Look, 2003) and notably is not expressed in normal T cells (McCormack et al., 2003). Previously an intracellular VH, VH576, binding to LMO2 (hereafter named iDAb LMO2) has been employed to show that T cell tumours do not grow when LMO2 is blocked (Tanaka et al., 2011) and showed the iDAb binds to LMO2 causing a stable structure that precludes the PPI with its natural partners (Sewell et al., 2014). In spite of this, there remains a need for compounds that bind to the same interface of the LMO2 as the iDAb LMO2 in order to interfere with LMO2 PPI in cells and modulate the activity of the T cell leukaemia chromosal translocation protein, LMO2.

In view of the relevance of the LMO2 protein for various physiological processes outlined above, inhibitors of the LMO2 protein such as the compounds of the present invention can be used in the treatment of various disease states in which LMO2 activity plays a role or which are associated with inappropriate LMO2 activity, or in which inhibition, regulation or modulation of signal transduction by the LMO2 protein is desired. Taken together these studies suggest selective inhibition of the LMO2 protein to be a promising therapeutic approach, particularly for the treatment of hyperproliferative diseases, such as cancer.

The present invention has been devised with the foregoing in mind.

### SUMMARY OF THE INVENTION

According to a first aspect of the present invention, there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting LMO2 activity, in vitro or in vivo, said method comprising contacting a cell with an effective amount of a compound or a pharmaceutically acceptable salt, hydrate or solvate thereof as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein.

According to a further aspect of the present invention, there is provided a method of treating a disease or disorder in which LMO2 activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition.

According to a further aspect of the present invention, there is provided a compound, or a pharmaceutically acceptable salt, hydrate or solvate thereof, or a pharmaceutical composition as defined herein for use in the treatment of cancer.

According to a further aspect of the present invention, there is provided a compound, or a pharmaceutically acceptable salt, hydrate or solvate thereof, as defined herein for use in the inhibition of LMO2 activity.

According to a further aspect of the present invention, there is provided a compound, or a pharmaceutically acceptable salt, hydrate or solvate thereof, as defined herein for use in the treatment of a disease or disorder in which LMO2 activity is implicated.

According to a further aspect of the present invention, there is provided the use of a compound, or a pharmaceutically acceptable salt, hydrate or solvate thereof, as defined herein in the manufacture of a medicament for the treatment of a proliferative condition.

Suitably, the proliferative disorder is cancer, suitably a human cancer. Particular examples of suitable cancers are any cancers in which LMO2 activity is implicated and include, but are not limited to, haematological cancers such as lymphomas (including diffuse large B-cell lymphoma (DLBCL), B-cell acute lymphoblastic lymphoma (B-ALL), follicular lymphoma (FL), Burkitt lymphoma (BL) and angioimmunoblastic T-cell lymphoma (AITL)), leukaemias (including acute lymphoblastic leukaemia (ALL), which includes T-cell acute lymphoblastic leukaemia (T-ALL), acute myeloid leukaemia (AML) and chronic myeloid leukaemia (CML)) and multiple myeloma.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided a use of a compound, or a pharmaceutically acceptable salt, hydrate or solvate thereof, as defined herein in the manufacture of a medicament for the inhibition of LMO2 activity.

According to a further aspect of the present invention, there is provided a use of a compound, or a pharmaceutically acceptable salt, hydrate or solvate thereof, as defined herein in the manufacture of a medicament for the treatment of a disease or disorder in which LMO2 activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), ethylene (—CH₂CH₂—), propylene (—CH₂CH₂CH₂—), 2-methylpropylene (—CH₂CH(CH₃)CH₂—), pentylene (—CH₂CH₂CH₂CH₂CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(3-10C)cycloalkyl” means a hydrocarbon ring containing from 3 to 10 carbon atoms, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups.

Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to compounds, or pharmaceutically acceptable salts, hydrates or solvates thereof, having the structural Formula (I), shown below:

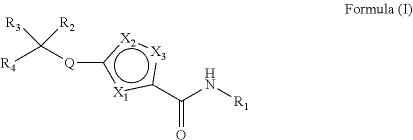

wherein:
R₁ is selected from:
   (i) (1-6C)alkyl which is optionally substituted by one or more Rₐ;
   (ii) a group of the formula:

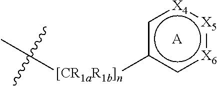

wherein
   [symbol] denotes the point of attachment;
   n is 0 or 1;
   R₁ₐ and R_{1b} are selected from hydrogen or methyl;
   X₄, X₅ and X₆ are selected from C—H, C—Rₐ or N;
(iii) a group of the formula:

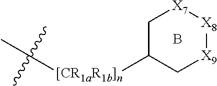

wherein
   [symbol] denotes the point of attachment;
   n, R₁ₐ and R_{1b} are as defined above;
   Ring B is a saturated or partially unsaturated ring;
   X₇, X₃ and X₉ are selected from C—H, C—Rₐ or N if a bond connecting them to an adjacent atom is an unsaturated double bond, or C—H₂, C—HRₐ, C—(Rₐ)₂, N—H, N—R_{b}, S or O if the bonds attaching them to adjacent atoms are single bonds;
wherein each Rₐ is independently selected from (1-4C)alkyl, halo, (1-4C)haloalkyl, (1-4C)haloalkoxy, cyano, nitro, (3-6C)cycloalkyl, (3-6C)cycloalkyl(1-2C)alkyl, phenyl, (CH₂)_{q1}NR_{ab}R_{ac}, (CH₂)_{q1}OR_{ab}, (CH₂)_{q1}C(O)R_{ab}, (CH₂)_{q1}C(O)OR_{ab}, (CH₂)_{q1}OC(O)R_{ab}, (CH₂)_{q1}C(O)N(R_{ac})R_{ab}, (CH₂)_{q1}N(R_{ac})C(O)R_{ab}, (CH₂)_{q1}S(O)ₚR_{ab} (where p is 0, 1 or 2), (CH₂)_{q1}SO₂N(R_{ac})R_{1ab}, or (CH₂)_{q1}N(R_{ac})SO₂R_{ab},
   and wherein:
   q1 is 0, 1, 2 or 3;
   R_{ab} is selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl and heterocyclyl(1-2C)alkyl,
      and wherein R_{ab} is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, nitro, NR_{ad}Rₐₑ, OR_{ad}, C(O)R_{ad}, C(O)OR_{ad}, OC(O)R_{ad}, C(O)N(Rₐₑ)R_{ad}, N(Rₐₑ)C(O)R_{ad}, S(O)ₚR_{ad} (where p is 0, 1 or 2), SO₂N(Rₐₑ)R_{ad}, N(Rₐₑ)SO₂R_{ad}, or (CH₂)_{q2}NR_{ad}Rₐₑ (where q2 is 1, 2 or 3); wherein R_{ad} and Rₐₑ are each independently selected from hydrogen or (1-6C)alkyl;
   and R_{ac} is selected from hydrogen or (1-2C)alkyl;
   or where R_{ab} and R_{ac} are linked to a common N atom, they may be linked such that, together with the N atom to which they are attached, they form a 5 or 6 membered heteroaryl ring or a 5 to 7 membered heterocyclic ring, each of which is optionally substituted as for R_{ab} above;
wherein R_{b} is independently selected from (1-4C)alkyl, (1-4C)haloalkyl or —C(O)R_{ba}, wherein R_{ba} is selected from (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl and heterocyclyl(1-2C)alkyl, and wherein R_{ba} is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, nitro, NR_{bd}R_{be}, OR_{bd}, C(O)R_{bd}, C(O)OR_{bd}, OC(O)R_{bd}, C(O)N(R_{be})R_{bd}, N(R_{be})C(O)R_{bd}, S(O)ₚR_{bd} (where p is 0, 1 or 2), SO₂N(R_{be})R_{bd}, N(R_{be})SO₂R_{bd}, or (CH₂)_{q3}NR_{bd}R_{be} (where q3 is 1, 2 or 3); wherein R_{b}d and R_{be} are each independently selected from hydrogen or (1-6C)alkyl;
X₁, X₂ and X₃ are selected from N, N—R₅, O, S and CR₆, wherein R₅ is hydrogen or methyl and R₆ is hydrogen, methyl or halo, with the proviso that at least one of X₁, X₂ and X₃ is selected from N, N—R₅, O and S;
Q is a group of the formula:

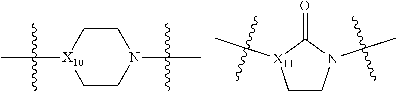

or —X₁₂—CH₂—CH₂—NR₇— or —X₁₃—C(O)—NR₈—;
wherein:
   X₁₀ and X₁₁ are selected from N or CH;
   X₁₂ and X₁₃ are selected from NR₉, CH₂, CHR₉ or C(R₉)₂; and
   R₇, R₈ and R₉ are selected from hydrogen or (1-2C)alkyl;
R₂ and R₃ are selected from hydrogen or (1-2C)alkyl;
R₄ is a phenyl, heteroaryl, or heterocyclyl ring optionally substituted by (1-4C)alkyl, halo, (1-4C)haloalkyl, (1-4C)haloalkoxy, (1-4C)aminoalkyl, (1-4C)hydroxyalkyl, cyano, nitro, NR₄ₐR_{4b}, OR₄ₐ, C(O)R₄ₐ, C(O)OR₄ₐ, OC(O)R₄ₐ, C(O)N(R_{4b})R₄ₐ, N(R_{4b})C(O)R₄ₐ, S(O)ₚR₄ₐ (where p is 0, 1 or 2), SO₂N(R_{4b})R₄ₐ, N(R_{4b})SO₂R₄ₐ, or (CH₂)_{q4}NR₄ₐR_{4b} (where q4 is 1, 2 or 3); wherein R₄ₐ is selected from hydrogen, (1-4C)alkyl, (3-6C)cycloalkyl, (3-6C)cycloalkyl(1-2C)alkyl, phenyl aryl(1-2C)alkyl, heteroaryl, heteroaryl(1-2C)alkyl, heterocyclyl and heterocyclyl(1-2C)alkyl, and wherein:
R₄ₐ is optionally further substituted by (1-4C)alkyl, halo, (1-4C)haloalkyl, (1-4C)haloalkoxy, cyano, nitro, NR₄ₐₐR_{4ab}, OR₄ₐₐ, C(O)R₄ₐₐ, C(O)OR₄ₐₐ, OC(O)R₄ₐₐ, C(O)N(R_{4ab})R₄ₐₐ, N(R_{4ab})C(O)R₄ₐₐ, S(O)ₚR₄ₐₐ (where p is 0, 1 or 2), SO₂N(R_{4ab})R₄ₐₐ, N(R_{4ab})SO₂R₄ₐₐ, or (CH₂)_{q5}NR₄ₐₐR_{4ab} (where q5 is 1, 2 or 3) and R₄ₐₐ and R_{4ab} are hydrogen or (1-2C)alkyl;
R_{4b} is selected from hydrogen or (1-2C)alkyl;
or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above.

Particular compounds of the invention include, for example, compounds of the Formula (I), or pharmaceutically acceptable salts, hydrates and/or solvates thereof, wherein, unless otherwise stated, each of R₁, X₁, X₂, X₃, Q, R₂, R₃ and R₄ and any associated substituent groups has any of the meanings defined hereinbefore or in any of paragraphs (1) to (22) hereinafter:
(1) R₁ is selected from:
   (i) (1-4C)alkyl which is optionally substituted by one or more Rₐ;
   (ii) a group of the formula:

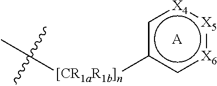

wherein
   [symbol] denotes the point of attachment;
   n is 0 or 1;
   R₁ₐ and R_{1b} are selected from hydrogen or methyl;
   X₄, X₅ and X₆ are selected from C—H, C—Rₐ or N;
(iii) a group of the formula:

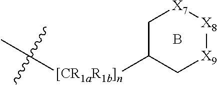

wherein
   [symbol] denotes the point of attachment;
   n, R₁ₐ and R_{1b} are as defined above;
   Ring B is a saturated or partially unsaturated ring;
   X₇, X₈ and X₉ are selected from C—H or C—Rₐ if a bond connecting them to an adjacent atom is an unsaturated double bond, or C—H₂, C—HRₐ, or C—(Rₐ)₂ if the bonds attaching them to adjacent atoms are single bonds;
wherein each Rₐ is independently selected from (1-2C)alkyl, halo, (1-2C)haloalkyl, (1-2C)haloalkoxy, cyano, nitro, (5-6C)cycloalkyl, (5-6C)cycloalkyl(1-2C)alkyl, phenyl, (CH₂)_{q1}NR_{ab}R_{ac}, (CH₂)_{q1}OR_{ab}, (CH₂)_{q1}C(O)R_{ab}, (CH₂)_{q1}C(O)OR_{ab}, (CH₂)_{q1}OC(O)R_{ab}, (CH₂)_{q1}C(O)N(R_{ac})R_{ab}, (CH₂)_{q1}N(R_{ac})C(O)R_{ab}, (CH₂)_{q1}S(O)ₚR_{ab} (where p is 0, 1 or 2), (CH₂)_{q1}SO₂N(R_{ac})R_{1ab}, or (CH₂)_{q1}N(R_{ac})SO₂R_{ab},
   and wherein:
   q1 is 0, 1 or 2;
   R_{ab} is selected from hydrogen, (1-4C)alkyl, (5-6C)cycloalkyl, (5-6C)cycloalkyl(1-2C)alkyl, aryl, aryl(1-2C)alkyl, heteroaryl, heteroaryl(1-2C)alkyl, heterocyclyl and heterocyclyl(1-2C)alkyl,
      and wherein R_{ab} is optionally further substituted by one or more substituent groups independently selected from oxo, (1-2C)alkyl, halo, (1-2C)haloalkyl, (1-2C)haloalkoxy, (1-2C)aminoalkyl, (1-2C)hydroxyalkyl, cyano, nitro, NR_{ad}Rₐₑ, OR_{ad}, C(O)R_{ad}, C(O)OR_{ad}, OC(O)R_{ad}, C(O)N(Rₐₑ)R_{ad}, N(Rₐₑ)C(O)R_{ad}, S(O)ₚR_{ad} (where p is 0, 1 or 2), SO₂N(Rₐₑ)R_{ad}, N(Rₐₑ)SO₂R_{ad}, or (CH₂)_{q2}NR_{ad}Rₐₑ (where q2 is 1, 2 or 3); wherein R_{ad} and Rₐₑ are each independently selected from hydrogen or (1-4C)alkyl;
   and Rₐe is selected from hydrogen or methyl;
   or where R_{ab} and R_{ac} are linked to a common N atom, they may be linked such that, together with the N atom to which they are attached, they form a 5 or 6 membered heteroaryl ring or a 5 to 7 membered heterocyclic ring, each of which is optionally substituted as for R_{ab} above;
(2) R₁ is selected from:
   (i) (1-4C)alkyl which is optionally substituted by one or more Rₐ;
   (ii) a group of the formula:

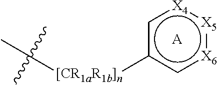

wherein
   [symbol] denotes the point of attachment;
   n is 0 or 1;
   R₁ₐ and R_{1b} are selected from hydrogen or methyl;
   X₄, X₅ and X₆ are selected from C—H, C—Rₐ or N;
(iii) a group of the formula:

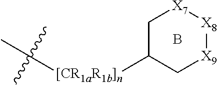

wherein
   [symbol] denotes the point of attachment;
   n, R₁ₐ and R_{1b} are as defined above;
   Ring B is a saturated or partially unsaturated ring;
   X₇, X₈ and X₉ are selected from C—H or C—Rₐ if a bond connecting them to an adjacent atom is an unsaturated double bond, or C—H₂, C—HRₐ, or C—(Rₐ)₂ if the bonds attaching them to adjacent atoms are single bonds;
wherein each Rₐ is independently selected from (1-2C)alkyl, halo, (1-2C)haloalkyl, cyano, nitro, (5-6C)cycloalkyl(1-2C)alkyl, phenyl, (CH₂)_{q1}NR_{ab}R_{ac}, (CH₂)_{q1}OR_{ab}, (CH₂)_{q1}C(O)R_{ab}, (CH₂)_{q1}C(O)OR_{ab}, (CH₂)_{q1}OC(O)R_{ab}, or (CH₂)_{q1}N(R_{ac})SO₂R_{ab},
   and wherein:
   q1 is 0, or 1;
   R_{ab} is selected from hydrogen, (1-4C)alkyl, (5-6C)cycloalkyl, (5-6C)cycloalkyl(1-2C)alkyl, aryl, aryl(1-2C)alkyl, heteroaryl and heterocyclyl(1-2C)alkyl,
      and wherein R_{ab} is optionally further substituted by one or more substituent groups independently selected from oxo, methyl, halo, cyano, nitro, NR_{ad}Rₐₑ, OR_{ad}, C(O)R_{ad}, C(O)OR_{ad}, OC(O)R_{ad}, C(O)N(Rₐₑ)R_{ad}, N(Rₐₑ)SO₂R_{ad}, or (CH₂)_{q2}NR_{ad}Rₐₑ (where q2 is 1 or 2); wherein R_{ad} and Rₐₑ are each independently selected from hydrogen or (1-2C)alkyl;
   and R_{ac} is selected from hydrogen or methyl;
   or where R_{ab} and R_{ac} are linked to a common N atom, they may be linked such that, together with the N atom to which they are attached, they form a 5 or 6 membered heteroaryl ring or a 5 to 7 membered heterocyclic ring, each of which is optionally substituted as for R_{ab} above;
(3) R₁ is selected from:
   (i) (1-4C)alkyl which is optionally substituted by one or more Rₐ;
   (ii) a group of the formula:

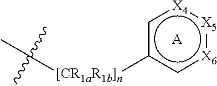

wherein
   [symbol] denotes the point of attachment;
   n is 0 or 1;
   R₁ₐ and R_{1b} are selected from hydrogen;
   X₄, X₅ and X₆ are selected from C—H, C—Rₐ or N;
(iii) a group of the formula:

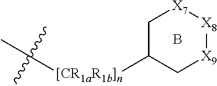

wherein
   [symbol] denotes the point of attachment;
   n, R₁ₐ and R_{1b} are as defined above;
   Ring B is a saturated ring;
   X₇, X₈ and X₉ are selected from C—H;
wherein each Rₐ is independently selected from methyl, halo, cyano, phenyl, (CH₂)_{q1}NR_{ab}R_{ac}, (CH₂)_{q1}OR_{ab}, (CH₂)_{q1}C(O)R_{ab} or (CH₂)_{q1}C(O)OR_{ab},
   and wherein:
   q1 is 0;
   R_{ab} is selected from hydrogen, (1-4C)alkyl, aryl, aryl(1-2C)alkyl,
      and wherein R_{ab} is optionally further substituted by one or more substituent groups independently selected from oxo, methyl, halo, or cyano;
      and R_{ac} is selected from hydrogen;
   or where R_{ab} and R_{ac} are linked to a common N atom, they may be linked such that, together with the N atom to which they are attached, they form a 5 or 6 membered heteroaryl ring or a 5 to 7 membered heterocyclic ring, each of which is optionally substituted as for R_{ab} above;
(4) R₁ is selected from:
   (i) (1-4C)alkyl which is optionally substituted by one or more Rₐ;
   (ii) a group of the formula:

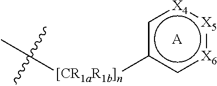

wherein
   [symbol] denotes the point of attachment:
   n is 0 or 1;
   R₁ₐ and R_{1b} are selected from hydrogen;
   X₄, X₅ and X₆ are selected from C—H, C—Rₐ or N;
(iii) a group of the formula:

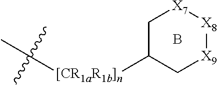

wherein
   [symbol] denotes the point of attachment;
   n, R₁ₐ and R_{1b} are as defined above;
   Ring B is a saturated ring;
   X₇, X₈ and X₉ are selected from C—H;
wherein each Rₐ is independently selected from methyl, halo, (CH₂)_{q1}NR_{ab}R_{ac}, (CH₂)_{q1}OR_{ab}, (CH₂)_{q1}C(O)R_{ab} or (CH₂)_{q1}C(O)OR_{ab},
   and wherein:
   q1 is 0;
   R_{ab} is selected from hydrogen, (1-4C)alkyl, aryl, aryl(1-2C)alkyl,
      and wherein R_{ab} is optionally further substituted by one or more substituent groups independently selected from, halo;
      and R_{ac} is selected from hydrogen;
   or where R_{ab} and R_{ac} are linked to a common N atom, they may be linked such that, together with the N atom to which they are attached, they form a 5 membered heteroaryl ring or a 5 or 6 membered heterocyclic ring, each of which is optionally substituted as for R_{ab} above;
(5) R₁ is selected from:
   (i) a group of the formula:

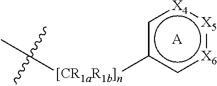

wherein
   [symbol] denotes the point of attachment;
   n is 0 or 1;
   R₁ₐ and R_{1b} are selected from hydrogen;
   X₄, X₅ and X₆ are selected from C—H, C—Rₐ or N;
(ii) a group of the formula:

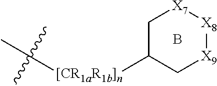

wherein
   [symbol] denotes the point of attachment;
   n is 0,
   R₁ₐ and R_{1b} are selected from hydrogen
   Ring B is a saturated ring;
   X₇, X₈ and X₉ are selected from C—H;
wherein each Rₐ is independently selected from methyl, halo, (CH₂)_{q1}NR_{ab}R_{ac}, (CH₂)_{q1}OR_{ab}, (CH₂)_{q1}C(O)R_{ab} or (CH₂)_{q1}C(O)OR_{ab},
   and wherein:
   q1 is 0;
   R_{ab} is selected from hydrogen, (1-2C)alkyl, phenyl, benzyl,
      and wherein R_{ab} is optionally further substituted by one or more substituent groups independently selected from, halo;
      and R_{ac} is selected from hydrogen;
   or where R_{ab} and R_{ac} are linked to a common N atom, they may be linked such that, together with the N atom to which they are attached, they form a 5 membered heteroaryl ring or a 5 or 6 membered heterocyclic ring, each of which is optionally substituted as for R_{ab} above;
(6) R₁ is selected from:

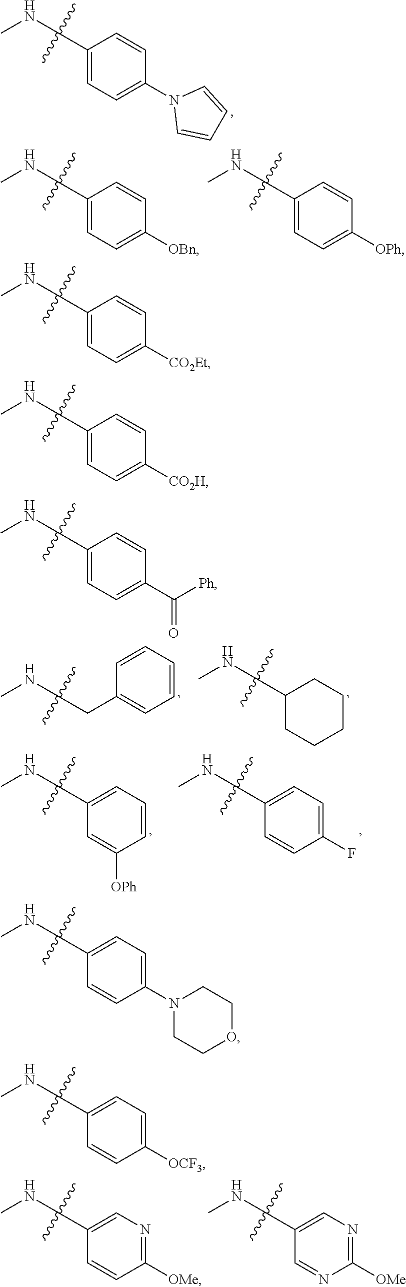

wherein
   [symbol] denotes the point of attachment;
(7) R₁ is selected from:

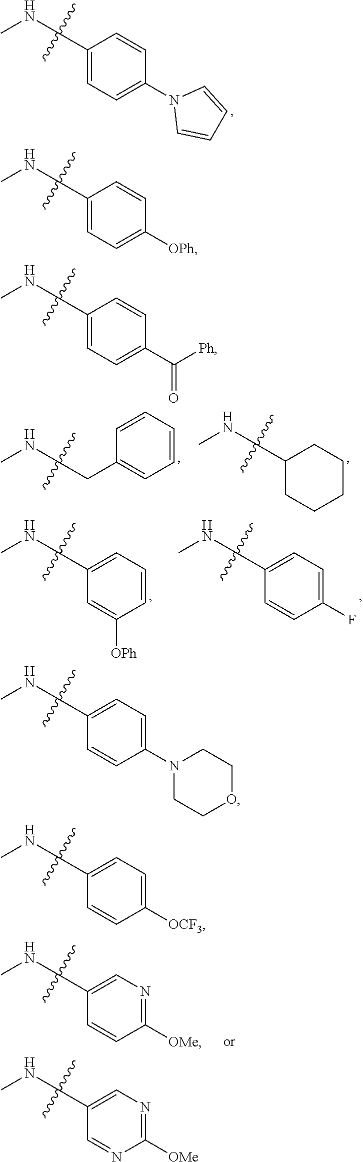

wherein
   [symbol] denotes the point of attachment;
(8) X₁, X₂ and X₃ are selected from N, O, S and CR₆, wherein R₆ is hydrogen, methyl or halo, with the proviso that at least one of X₁, X₂ and X₃ is selected from N, O and S;
(9) X₁, X₂ and X₃ are selected from N, O, S and CR₆, wherein R₆ is hydrogen, with the proviso that at least one of X₁, X₂ and X₃ is selected from N, O and S;
(10) X₁ is N; X₂ is O or S; and X₃ is CR₆, wherein R₆ is hydrogen;
(11) Q is a group of the formula:

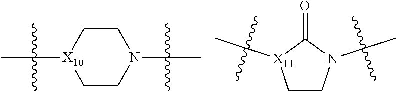

or —X₁₂—CH₂—CH₂—NR₇— or —X₁₃—C(O)—NR₈—;
wherein:
X₁₀ and X₁₁ are selected from N or CH;
X₁₂ and X₁₃ are selected from NR₉ and CH₂; and
R₇, R₈ and R₉ are selected from hydrogen or methyl;
wherein
   [symbol] denotes the point of attachment;
(12) Q is a group of the formula:

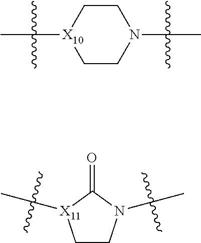

or —X₁₂—CH₂—CH₂—NR₇— or —X₁₃—C(O)—NR₈—;
wherein:
X₁₀ and X₁₁ are selected from N;
X₁₂ and X₁₃ are selected from NR₉; and
R₇, R₈ and R₉ are selected from hydrogen;
wherein [symbol] denotes the point of attachment;
(13) Q is a group of the formula:

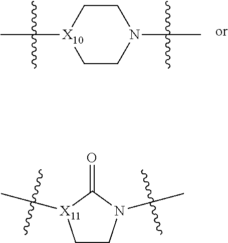

wherein:
X₁₀ and X₁₁ are selected from N;
wherein [symbol] denotes the point of attachment;
(14) Q is selected from

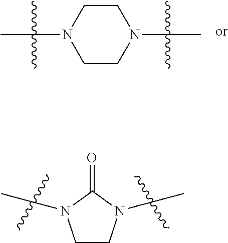

wherein [symbol] denotes the point of attachment;
(15) R₂ and R₃ are selected from hydrogen or methyl;
(16) R₂ and R₃ are hydrogen;
(17) R₄ is a phenyl, heteroaryl, or heterocyclyl ring optionally substituted by (1-2C)alkyl, halo, (1-2C)haloalkyl, (1-2C)haloalkoxy, (1-2C)aminoalkyl, (1-2C)hydroxyalkyl, cyano, nitro, NR₄ₐR_{4b}, OR₄ₐ, C(O)R₄ₐ, C(O)OR₄ₐ, OC(O)R₄ₐ, C(O)N(R_{4b})R₄ₐ, N(R_{4b})C(O)R₄ₐ, S(O)ₚR₄ₐ (where p is 0, 1 or 2), SO₂N(R_{4b})R₄ₐ, N(R_{4b})SO₂R₄ₐ, or (CH₂)_{q4}NR₄ₐR_{4b} (where q4 is 1, 2 or 3); wherein R₄ₐ is selected from hydrogen, (1-2C)alkyl, (5-6C)cycloalkyl, (5-6C)cycloalkyl(1-2C)alkyl, phenyl, phenyl(1-2C)alkyl, heteroaryl, heteroaryl(1-2C)alkyl, heterocyclyl and heterocyclyl(1-2C)alkyl, and wherein:
   R₄ₐ is optionally further substituted by (1-2C)alkyl, halo, (1-2C)haloalkyl, (1-2C)haloalkoxy, cyano, nitro, NR₄ₐₐR_{4ab}, OR₄ₐₐ, C(O)R₄ₐₐ, C(O)OR₄ₐₐ, OC(O)R₄ₐₐ, C(O)N(R_{4ab})R₄ₐₐ, N(R_{4ab})C(O)R₄ₐₐ, S(O)ₚR₄ₐₐ (where p is 0, 1 or 2), SO₂N(R_{4ab})R₄ₐₐ, N(R_{4ab})SO₂R₄ₐₐ, or (CH₂)_{q5}NR₄ₐₐR_{4ab} (where q5 is 1, 2 or 3) and R₄ₐₐ and R_{4ab} are hydrogen or (1-2C)alkyl;
   R_{4b} is selected from hydrogen or (1-2C)alkyl;
   or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above;
(18) R₄ is a phenyl, heteroaryl, or heterocyclyl ring optionally substituted by (1-2C)alkyl, halo, (1-2C)haloalkyl, (1-2C)haloalkoxy, (1-2C)aminoalkyl, (1-2C)hydroxyalkyl, cyano, nitro, NR₄ₐR_{4b}, OR₄ₐ, C(O)R₄ₐ, C(O)OR₄ₐ, OC(O)R₄ₐ, C(O)N(R_{4b})R₄ₐ, N(R_{4b})C(O)R₄ₐ, S(O)ₚR₄ₐ (where p is 0, 1 or 2), SO₂N(R_{4b})R₄ₐ, N(R_{4b})SO₂R₄ₐ, or (CH₂)_{q4}NR₄ₐR_{4b} (where q4 is 1, 2 or 3); wherein R₄ₐ is selected from hydrogen, (1-2C)alkyl, phenyl aryl(1-2C)alkyl, heteroaryl, heteroaryl(1-2C)alkyl, heterocyclyl and heterocyclyl(1-2C)alkyl, and wherein:
   R₄ₐ is optionally further substituted by (1-2C)alkyl, halo, (1-2C)haloalkyl, (1-2C)haloalkoxy, cyano, nitro;
   R_{4b} is selected from hydrogen or (1-2C)alkyl;
   or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above;
(19) R₄ is a phenyl, heteroaryl, or heterocyclyl ring optionally substituted by halo, cyano, nitro, NR₄ₐR_{4b}, OR₄ₐ, C(O)R₄ₐ, N(R_{4b})C(O)R₄ₐ; wherein R₄ₐ is selected from hydrogen, (1-2C)alkyl, phenyl aryl(1-2C)alkyl;
   R_{4b} is selected from hydrogen or methyl;
   or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above;
(20) R₄ is a phenyl ring optionally substituted by halo, cyano, nitro, NR₄ₐR_{4b}, OR₄ₐ, C(O)R₄ₐ, N(R_{4b})C(O)R₄ₐ; wherein R₄ₐ is selected from hydrogen, (1-2C)alkyl, phenyl aryl(1-2C)alkyl;
   R_{4b} is selected from hydrogen;
(21) R₄ is selected from

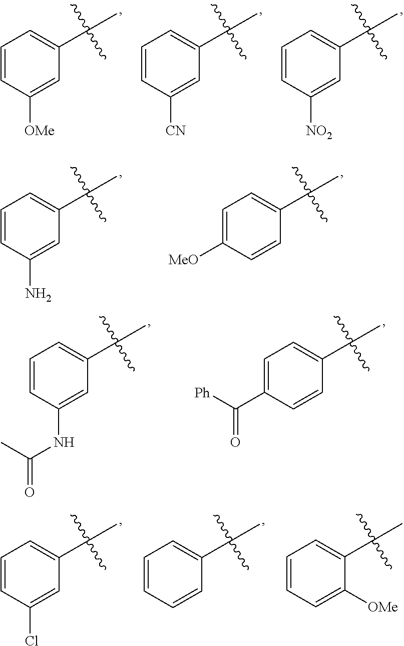

wherein
   [symbol] denotes the point of attachment;
(22) Rₐ is selected from

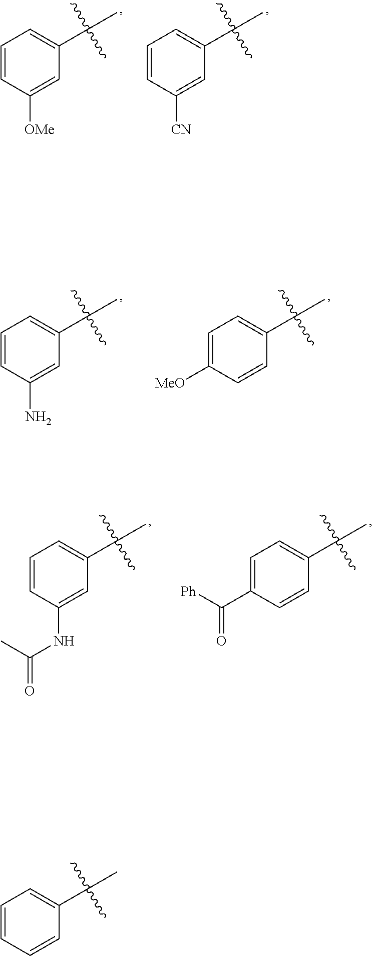

wherein
   [symbol] denotes the point of attachment.

Suitably, R₁ is as defined in any one of paragraphs (4) to (7) above. Most suitably, R₁ is as defined in paragraph (6) or paragraph (7) above.

Suitably, X₁, is as defined in any one of paragraphs (8) to (10) above. Most suitably, X₁, is as defined in paragraph (10) above.

Suitably, X₂ is as defined in any one of paragraphs (8) to (10) above. Most suitably, X₂ is as defined in paragraph (10) above.

Suitably, X₃ is as defined in any one of paragraphs (8) to (10) above. Most suitably, X₃ is as defined in paragraph (10) above.

Suitably, X₁, X₂ and X₃ are as defined in any one of paragraphs (8) to (10) above.

Most suitably, X₁, X₂ and X₃ are as defined in paragraph (10) above.

Suitably, Q is as defined in any one of paragraphs (11) to (14) above. Most suitably, Q is as defined in paragraph (14) above.

Suitably, R₂ is as defined in paragraphs (15) and (16) above. Most suitably, R₂ is as defined in paragraph (16) above.

Suitably, R₃ is as defined in paragraphs (15) and (16) above. Most suitably, R₃ is as defined in paragraph (16) above.

Suitably R₂ and R₃ are as defined in paragraphs (15) and (16) above. Most suitably, R₂ and R₃ is as defined in paragraph (16) above.

Suitably, R₄ is as defined in any one of paragraphs (19) to (22) above. Most suitably, R₄ is as defined in paragraph (21) or paragraph (22) above.

In a particular group of compounds of the invention, R₂ and R₃ are hydrogen and Q is

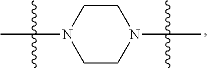

wherein [symbol] denotes the point of attachment,
the compounds have the structural formula Ia (a sub-definition of Formula (I)) shown below, or a pharmaceutically acceptable salt, hydrate and/or solvate thereof:

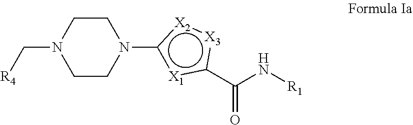

wherein each of R₁, X₁, X₂, X₃, and R₄ are as defined hereinabove.

In an embodiment of the compounds of Formula Ia:
R₁ is as defined in any one of paragraphs (1) to (7) above;
X₁, X₂, and X₃ are as defined in any one of paragraphs (8) to (10) above; and
R₄ is as defined in any one of paragraphs (17) to (22) above.

In another embodiment of the compounds of Formula Ia:
R₁ is as defined in paragraph (4) above;
X₁, X₂, and X₃ are as defined in paragraph (8) above; and
R₄ is as defined in paragraph (18) above.

In another embodiment of the compounds of Formula Ia:
R₁ is as defined in paragraph (5) above;
X₁, X₂, and X₃ are as defined in paragraph (9) above; and
R₄ is as defined in paragraph (19) above.

In another embodiment of the compounds of Formula Ia:
R₁ is as defined in paragraph (6) or paragraph (7) above;
X₁, X₂, and X₃ are as defined in paragraph (10) above; and
R₄ is as defined in paragraph (21) or paragraph (22) above.

In another embodiment of the compounds of Formula Ia:
R₁ is as defined in paragraph (6) above;
X₁, X₂, and X₃ are as defined in paragraph (10) above; and
R₄ is as defined in paragraph (21) above.

In another embodiment of the compounds of Formula Ia:
R₁ is as defined in paragraph (7) above;
X₁, X₂, and X₃ are as defined in paragraph (10) above; and
R₄ is as defined in paragraph (22) above.

In a particular group of compounds of the invention, the compounds have structural formula Ib (a sub-definition of Formula (I)) shown below, or a pharmaceutically acceptable salt, hydrate and/or solvate thereof:

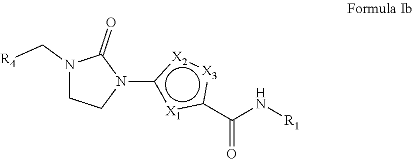

wherein each of R₁, X₁, X₂, X₃, and R₄ are as defined hereinabove.

In an embodiment of the compounds of Formula Ib:
R₁ is as defined in any one of paragraphs (1) to (7) above;
X₁, X₂, and X₃ are as defined in any one of paragraphs (8) to (10) above; and
R₄ is as defined in any one of paragraphs (17) to (22) above.

In another embodiment of the compounds of Formula Ib:
R₁ is as defined in paragraph (4) above;
X₁, X₂, and X₃ are as defined in paragraph (8) above; and
R₄ is as defined in paragraph (18) above.

In another embodiment of the compounds of Formula Ib:
R₁ is as defined in paragraph (5) above;
X₁, X₂, and X₃ are as defined in paragraph (9) above; and
R₄ is as defined in paragraph (19) above.

In another embodiment of the compounds of Formula Ib:
R₁ is as defined in paragraph (6) or paragraph (7) above;
X₁, X₂, and X₃ are as defined in paragraph (10) above; and
R₄ is as defined in paragraph (21) or paragraph (22) above.

In another embodiment of the compounds of Formula Ib:
R₁ is as defined in paragraph (6) above;
X₁, X₂, and X₃ are as defined in paragraph (10) above; and
R₄ is as defined in paragraph (21) above.

In another embodiment of the compounds of Formula Ib:
R₁ is as defined in paragraph (7) above;
X₁, X₂, and X₃ are as defined in paragraph (10) above; and
R₄ is as defined in paragraph (22) above.

In a particular group of compounds of the invention, the compounds have structural formula Ic (a sub-definition of Formula (I)) shown below, or a pharmaceutically acceptable salt, hydrate and/or solvate thereof:

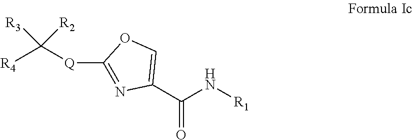

wherein each of R₁, Q, R₂, R₃ and R₄ are as defined hereinabove.

In an embodiment of the compounds of Formula Ic:
R₁ is as defined in any one of paragraphs (1) to (7) above;
Q is as defined in any one of paragraphs (11) to (14) above;
R₂ and R₃ are as defined in paragraphs (15) and (16) above; and
R₄ is as defined in any one of paragraphs (17) to (22) above.

In another embodiment of the compounds of Formula Ic:
R₁ is as defined in paragraph (4) above;
Q is as defined in paragraph (12) above;
R₂ and R₃ are as defined in paragraph (15) above; and
R₄ is as defined in paragraph (18) above.

In another embodiment of the compounds of Formula Ic:
R₁ is as defined in paragraph (5) above;
Q is as defined in paragraph (12) above;
R₂ and R₃ are as defined in paragraph (15) above; and
R₄ is as defined in paragraph (19) above.

In another embodiment of the compounds of Formula Ic:
R₁ is as defined in paragraphs (6) or paragraph (7) above;
Q is as defined in paragraph (14) above;
R₂ and R₃ are as defined in paragraph (16) above; and
R₄ is as defined in paragraph (21) or paragraph (22) above.

In another embodiment of the compounds of Formula Ic:
R₁ is as defined in paragraph (6) above;
Q is as defined in paragraph (14) above;
R₂ and R₃ are as defined in paragraph (16) above; and
R₄ is as defined in paragraph (21) above.

In another embodiment of the compounds of Formula Ic:
R₁ is as defined in paragraph (7) above;
Q is as defined in paragraph (14) above;
R₂ and R₃ are as defined in paragraph (16) above; and
R₄ is as defined in paragraph (22) above.

In a particular group of compounds of the invention, the compounds have structural formula Id (a sub-definition of Formula (I)) shown below, or a pharmaceutically acceptable salt, hydrate and/or solvate thereof:

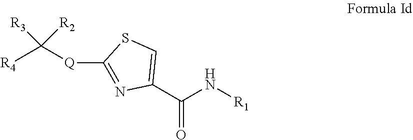

wherein each of R₁, Q, R₂, R₃ and R₄ are as defined hereinabove.

In an embodiment of the compounds of Formula Id:
R₁ is as defined in any one of paragraphs (1) to (7) above;
Q is as defined in any one of paragraphs (11) to (14) above;
R₂ and R₃ are as defined in paragraphs (15) and (16) above; and
R₄ is as defined in any one of paragraphs (17) to (22) above.

In another embodiment of the compounds of Formula Id:
R₁ is as defined in paragraph (4) above;
Q is as defined in paragraph (12) above;
R₂ and R₃ are as defined in paragraph (15) above; and
R₄ is as defined in paragraph (18) above.

In another embodiment of the compounds of Formula Id:
R₁ is as defined in paragraph (5) above;
Q is as defined in paragraph (12) above;
R₂ and R₃ are as defined in paragraph (15) above; and
R₄ is as defined in paragraph (19) above.

In another embodiment of the compounds of Formula Id:
R₁ is as defined in paragraphs (6) or paragraph (7) above;
Q is as defined in paragraph (14) above;
R₂ and R₃ are as defined in paragraph (16) above; and
R₄ is as defined in paragraph (21) or paragraph (22) above.

In another embodiment of the compounds of Formula Id:
R₁ is as defined in paragraph (6) above;
Q is as defined in paragraph (14) above;
R₂ and R₃ are as defined in paragraph (16) above; and
R₄ is as defined in paragraph (21) above.

In another embodiment of the compounds of Formula Id:
R₁ is as defined in paragraph (7) above;
Q is as defined in paragraph (14) above;
R₂ and R₃ are as defined in paragraph (16) above; and
R₄ is as defined in paragraph (22) above.

In a particular group of compounds of the invention, X₁, X₂ and X₃ are as defined in paragraph (10) above, Q is as defined in paragraph (14) above, R₂ and R₃ are as defined in paragraph (15) above and:
R₁ is as defined in any one of paragraphs (1) to (7) above;
R₄ is as defined in any one of paragraphs (17) to (22) above.

In a further group of compounds of the invention, X₁, X₂ and X₃ are as defined in paragraph (10) above, Q is as defined in paragraph (14) above, R₂ and R₃ are as defined in paragraph (15) above and:
R₁ is as defined in paragraph (5) above; and
R₄ is as defined in paragraph (18) above.

In a further group of compounds of the invention, X₁, X₂ and X₃ are as defined in paragraph (10) above, Q is as defined in paragraph (14) above, R₂ and R₃ are as defined in paragraph (15) above and:
R₁ is as defined in paragraph (5) above; and
R₄ is as defined in paragraph (19) above.

In a further group of compounds of the invention, X₁, X₂ and X₃ are as defined in paragraph (10) above, Q is as defined in paragraph (14) above, R₂ and R₃ are as defined in paragraph (15) above and:
R₁ is as defined in paragraph (6) above; and
R₄ is as defined in paragraph (20) above.

In a further group of compounds of the invention, X₁, X₂ and X₃ are as defined in paragraph (10) above, Q is as defined in paragraph (14) above, R₂ and R₃ are as defined in paragraph (15) above and:
R₁ is as defined in paragraph (6) above; and
R₄ is as defined in paragraph (21) above.

In a further group of compounds of the invention, X₁, X₂ and X₃ are as defined in paragraph (10) above, Q is as defined in paragraph (14) above, R₂ and R₃ are as defined in paragraph (15) above and:
R₁ is as defined in paragraph (7) above; and
R₄ is as defined in paragraph (22) above.

Particular compounds of the present invention include any of the compounds exemplified in the present application, or a pharmaceutically acceptable salt or solvate thereof, and, in particular, any of the following:
2-(3-benzyl-2-oxoimidazolidin-1-yl)-N-(4-phenoxyphenyl)oxazole-4-carboxamide (Abd-L6);
N-(4-(1H-pyrrol-1-yl)phenyl)-2-(3-(3-chlorobenzyl)-2-oxoimidazolidin-1-yl)oxazole-4-carboxamide (Abd-L7);
2-(3-(4-chlorobenzyl)-2-oxoimidazolidin-1-yl)-N-(3,4-dimethoxyphenyl)oxazole-5-carboxamide (Abd-L8);
N-(4-(benzyloxy)phenyl)-2-(3-(3-methoxybenzyl)-2-oxoimidazolidin-1-yl)oxazole-4-carboxamide (Abd-L9);
N-(4-(1H-pyrrol-1-yl)phenyl)-2-(3-(3-cyanobenzyl)-2-oxoimidazolidin-1-yl)oxazole-4-carboxamide (Abd-L10);
2-(3-(2-chlorobenzyl)-2-oxoimidazolidin-1-yl)-N-(4-phenoxyphenyl)oxazole-4-carboxamide (Abd-L12);
2-(3-benzyl-2-oxoimidazolidin-1-yl)-N-(4-phenoxybenzyl)oxazole-4-carboxamide (Abd-L13);
2-(3-(2-chlorobenzyl)-2-oxoimidazolidin-1-yl)-N-(4-phenoxybenzyl)oxazole-4-carboxamide (Abd-L14);
N-(4-(1H-pyrrol-1-yl)phenyl)-2-(4-(3-methoxybenzyl)piperazin-1-yl)oxazole-4-carboxamide (Abd-L15);
N-(4-(benzyloxy)phenyl)-2-(4-(3-methoxybenzyl)piperazin-1-yl)oxazole-4-carboxamide (Abd-L16);
N-(4-(benzyloxy)phenyl)-2-(4-(3-methoxybenzyl)piperazin-1-yl)thiazole-4-carboxamide (Abd-L17);
N-(4-(1H-pyrrol-1-yl)phenyl)-2-(4-(3-methoxybenzyl)piperazin-1-yl)thiazole-4-carboxamide (Abd-L18);
N-(4-(1H-pyrrol-1-yl)phenyl)-2-(4-(4-methoxybenzyl)piperazin-1-yl)oxazole-4-carboxamide (Abd-L19);
N-(4-(1H-pyrrol-1-yl)phenyl)-2-(4-(2-methoxybenzyl)piperazin-1-yl)thiazole-4-carboxamide (Abd-L20);
2-(4-(3-methoxybenzyl)piperazin-1-yl)-N-(4-(trifluoromethoxy)phenyl)thiazole-4-carboxamide (Abd-L21);
2-(4-(3-methoxybenzyl)piperazin-1-yl)-N-(6-methoxypyridin-3-yl)thiazole-4-carboxamide (Abd-L22); and
2-(4-(3-methoxybenzyl)piperazin-1-yl)-N-(2-methoxypyrimidin-5-yl)thiazole-4-carboxamide (Abd-L23).

The various functional groups and substituents making up the compounds of the Formula (I), or sub-formulae Ia to Id, are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the Formula (I), or sub-formulae Ia to Id, may exist in solvated as well as unsolvated forms such as, for example, hydrated forms. It is to be understood that the invention encompasses all such solvated forms that possess antiproliferative activity.

It is also to be understood that certain compounds of the Formula (I), or sub-formulae Ia to Id, may exhibit polymorphism, and that the invention encompasses all such forms that possess antiproliferative activity.

Compounds of the Formula (I), or sub-formulae Ia to Id, may exist in a number of different tautomeric forms and references to compounds of the Formula (I), or sub-formulae Ia to Id, include all such forms. For the avoidance of doubt, where a compound can exist in one of several tautomeric forms, and only one is specifically described or shown, all others are nevertheless embraced by Formula (I), or sub-formulae Ia to Id. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

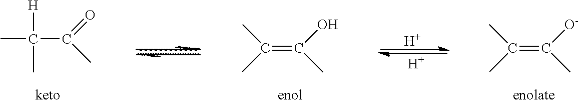

Compounds of the Formula (I), or sub-formulae Ia told, containing an amine function may also form N-oxides. A reference herein to a compound of the Formula (I), or sub-formulae Ia to Id, that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae Ia to Id, may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae Ia to Id, and in-vivo cleavable amide derivatives that may be formed at a carboxy group or an amino group in a compound of the Formula (I), or sub-formulae Ia to Id.

Accordingly, the present invention includes those compounds of the Formula (I), or sub-formulae Ia to Id, as defined hereinbefore, when made available by organic synthesis and when made available within the human or animal body by way of cleavage of a pro-drug thereof. Accordingly, the present invention includes those compounds of the Formula (I), or sub-formulae Ia to Id, that are produced by organic synthetic means and also such compounds that are produced in the human or animal body by way of metabolism of a precursor compound, that is a compound of the Formula (I), or sub-formulae Ia to Id, may be a synthetically-produced compound or a metabolically-produced compound.

A suitable pharmaceutically acceptable pro-drug of a compound of the Formula (I), or sub-formulae Ia to Id, is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae Ia to Id, that possesses a carboxy group is, for example, an in vivo cleavable ester thereof. An in vivo cleavable ester of a compound of the Formula I, or sub-formulae Ia to Id, containing a carboxy group is, for example, a pharmaceutically acceptable ester which is cleaved in the human or animal body to produce the parent acid. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae Ia to Id, that possesses a hydroxy group is, for example, an in vivo cleavable ester or ether thereof. An in vivo cleavable ester or ether of a compound of the Formula (I), or sub-formulae Ia to Id, containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I), or sub-formulae Ia to Id, that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I), or sub-formulae Ia to Id, that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae Ia to Id, may be exerted in part by one or more metabolites that are formed within the human or animal body after administration of a compound of the Formula (I), or sub-formulae Ia to Id. As stated hereinbefore, the in vivo effects of a compound of the Formula (I), or sub-formulae Ia to Id, may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae Ia to Id, will vary depending on the nature of R₁, X₁, X₂, X₃, Q, R₂, R₃ and R₄ and any substituent groups associated therewith. Suitable processes for their preparation are described further in the accompanying Examples.

Once a compound of Formula (I), or sub-formulae Ia to Id, has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups R₁, X₁, X₂, X₃, Q, R₂, R₃ and R₄ may be further reacted to change the nature of the group and provide an alternative compound of Formula (I).

The resultant compounds of Formula (I), or sub-formulae Ia to Id, can be isolated and purified using techniques well known in the art.

The compounds of Formula (I) may be synthesised by the general synthetic routes shown in the Examples section below, specific examples of which are described in more detail in the Examples.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the LMO2 in vitro assay described in the Examples section.

In general, as illustrated by the Example compound data in Table 1, the compounds of the invention demonstrate an IC₅₀ of 17 μM or less with the exception of Abd-L19 in the LMO2-iDAb LMO2_{dm3} BRET assay described in the examples section. Preferred compounds of the invention, such as Abd-L9, Abd-L10 and Abd-L16 demonstrate an IC₅₀ of 2 μM or less in the LMO2-iDAb LMO2_{dm3} BRET assay.

Also illustrated by the Example compound data in Table 1, a compound of the invention demonstrated an IC₅₀ of 2 μM or less in the LMO2-LDB1 BRET assay described in the examples section.

The following BRET assay data were generated for the Examples:

TABLE 1

| | LMO2 - iDAb LMO2_{dm3} | |
|---|---|---|
| Compound ID | IC₅₀ (μM) | % of inhibition at 50 mM |
| Abd-L9 | 1.5 | 46 |
| Abd-L10 | 1.0 | 42 |
| Abd-L15 | 16.6 | 53 |
| Abd-L16 | 1.2 | 44 |
| Abd-L17 | 13.0 | 49 |
| Abd-L18 | 15.8 | 55 |
| Abd-L19 | 48.8 | 52 |
| Abd-L22 | 6.8 | 85 |
| Compound ID | LMO2 - LDB1 IC₅₀ (μM) | % of inhibition at 50 mM |
| Abd-L10 | 1.2 | 35 |

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of LMO2 activity.

The present invention therefore provides a method of inhibiting LMO2 activity in vitro or in vivo, said method comprising contacting a cell with an effective amount of a compound, or a pharmaceutically acceptable salt, hydrate or solvate thereof, as defined herein.

The present invention also provides a method of treating a disease or disorder in which LMO2 activity is implicated in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of inhibiting cell proliferation, in vitro or in vivo, said method comprising contacting a cell with an effective amount of a compound, or a pharmaceutically acceptable salt, hydrate or solvate thereof, as defined herein.

The present invention provides a method of treating a proliferative disorder in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of treating cancer in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use in the treatment of a proliferative condition.

The present invention provides a compound, or a pharmaceutically acceptable salt, hydrate or solvate thereof, or a pharmaceutical composition as defined herein for use in the treatment of cancer.

The present invention provides a compound, or a pharmaceutically acceptable salt, hydrate or solvate thereof, as defined herein, for use in the inhibition of LMO2 activity.

The present invention provides a compound, or a pharmaceutically acceptable salt, hydrate or solvate thereof, as defined herein for use in the treatment of a disease or disorder in which LMO2 activity is implicated.

The present invention provides a use of a compound, or a pharmaceutically acceptable salt, hydrate or solvate thereof, as defined herein in the manufacture of a medicament for the treatment of a proliferative condition.

The present invention provides a use of a compound, or a pharmaceutically acceptable salt, hydrate or solvate thereof, as defined herein in the manufacture of a medicament for the treatment of cancer.

The present invention provides a use of a compound, or a pharmaceutically acceptable salt, hydrate or solvate thereof, as defined herein in the manufacture of a medicament for the inhibition of LMO2 activity.

The present invention provides a use of a compound, or a pharmaceutically acceptable salt, hydrate or solvate thereof, as defined herein in the manufacture of a medicament for the treatment of a disease or disorder in which LMO2 activity is implicated.

The term “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers (including breast cancer, non-small cell lung cancer (NSCLC) and squamous cell carcinomas (SCC) (including SCC of the head and neck, oesophagus, lung and ovary), lymphomas (including diffuse large B-cell lymphoma (DLBCL), B-cell acute lymphoblastic lymphoma (B-ALL), follicular lymphoma (FL), Burkitt lymphoma (BL) and angioimmunoblastic T-cell lymphoma (AITL)), leukaemias (including acute lymphoblastic leukaemia (ALL), which includes T-cell acute lymphoblastic leukaemia (T-ALL), acute myeloid leukaemia (AML) and chronic myeloid leukaemia (CML)), multiple myeloma lymphomas (including acute lymphoblastic leukaemia (ALL) and chronic myeloid leukaemia (CML)), psoriasis, bone diseases, fibroproliferative disorders (e.g., of connective tissues), and atherosclerosis. Any type of cell may be treated, including but not limited to, lymphatic, blood, lung, colon, breast, ovarian, prostate, liver, pancreas, brain, and skin.

Particular proliferative disorders of interest are haematological cancers, such as, for example, lymphomas (including diffuse large B-cell lymphoma (DLBCL), B-cell acute lymphoblastic lymphoma (B-ALL), follicular lymphoma (FL), Burkitt lymphoma (BL) and angioimmunoblastic T-cell lymphoma (AITL)), leukaemias (including acute lymphoblastic leukaemia (ALL), which includes T-cell acute lymphoblastic leukaemia (T-ALL), acute myeloid leukaemia (AML) and chronic myeloid leukaemia (CML)) and multiple myeloma. Diffuse large B-cell lymphoma (DLBCL), B-cell acute lymphoblastic lymphoma (B-ALL), angioimmunoblastic T-cell lymphoma (AITL), T-cell acute lymphoblastic leukaemia (T-ALL), and acute myeloid leukaemia (AML) are of particular interest.

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The compound of Formula (I), or a pharmaceutically acceptable salt thereof, being an inhibitor of LMO2, has potential therapeutic uses in a variety of LMO2-mediated disease states.

According to a further aspect of the specification there is provided a compound of Formula (I), or a pharmaceutically acceptable salt thereof, as defined hereinbefore for use in the treatment of cancer.

According to a further feature of this aspect of the specification there is provided a method for treating cancers in a warm-blooded animal, such as man, that is in need of such treatment, which comprises administering an effective amount of a compound of Formula (I), or a pharmaceutically acceptable salt thereof, as defined hereinbefore.

According to a further feature of this aspect of the specification there is provided the use of a compound of Formula (I), or a pharmaceutically acceptable salt thereof, as defined hereinbefore in the manufacture of a medicament for use in the treatment of cancers.

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5a-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan; (viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy, wherein the chemotherapy may include one or more anti-tumour agents selected from cyclophosphamide, epirubicin, fluorouracil, methotrexate, mitomycin C, doxorubicin, gemcitabine, docetaxel, carbazitaxel and radium-223 dichloride.

In another particular embodiment, the antiproliferative treatment defined hereinbefore may involve, in addition to the compound of the invention, conventional surgery or radiotherapy or chemotherapy, wherein the chemotherapy may include one or more anti-hormonal agents selected from a selective estrogen receptor modulator (SERM) (e.g. tamoxifen or toremifene), an aromatase inhibitor (AI) (e.g. anastrozole, fadrozole, letrozole or exemestane), a selective estrogen receptor degrader (SERD) (e.g. fulvestrant, elacestrant or GDC-0810), a luteinising hormone (LH) blocker (e.g. goserelin), direct androgen receptor (AR) antagonist (e.g. bicalutamide, enzalutamide, apalutamide, darolutamide, cyproterone acetate or flutamide), a non-competitive AR antagonist (e.g. ralaniten acetate), a androgen steroid synthesis inhibitor (e.g. abiraterone acetate), a gonadotropin-releasing hormone (GNRH) modulator (e.g. leuprorelin, goserelin, buserelin, triptorelin, degarelix).

In another particular embodiment, the antiproliferative treatment defined hereinbefore may involve, in addition to the compound of the invention, conventional surgery or radiotherapy or chemotherapy, wherein the chemotherapy may include one or cell-cycle agents selected from a cyclin-dependent kinase 4/6 (CDK4/6) inhibitor (e.g. palbociclib, ribociclib or abemaciclib).

In another particular embodiment, the antiproliferative treatment defined hereinbefore may involve, in addition to the compound of the invention, conventional surgery or radiotherapy or chemotherapy, wherein the chemotherapy may include one or DNA damage response agents agents selected from a poly ADP ribose polymerase (PARP) inhibitor (e.g. olaparib, veliparib, rucaparib or niraparib).

In another particular embodiment, the antiproliferative treatment defined hereinbefore may involve, in addition to the compound of the invention, conventional surgery or radiotherapy or chemotherapy, wherein the chemotherapy may include one or cell signalling agent selected from a phosphatidylinositol 3 kinase (PI3K) inhibitor, (e.g. buparlisib, apitolisib, azd8186, omipalisib, duvelisib, gedatolisib, copanlisib, pictilisib, alpelisib, idelalisib, acalisib, serabelisib, pilaralisib or taselisib), an AKT inhibitor (e.g. MK2206, AZD5363, afuresertib, AT13148, miransertib or ipatasertib); a (mTOR) signaling pathway inhibitor (e.g. everolimus, sirolimus, temsirolimus, vistusertib, sapanisertib or ridaforolimus), an Fibroblast growth factor (FGF) signalling inhibitor (e.g. AZD4547 or dovitinib).

In a particular embodiment, the antiproliferative treatment defined hereinbefore may involve, in addition to the compound of the invention, conventional surgery or radiotherapy or chemotherapy, wherein the chemotherapy may include one or more anti-tumour agents selected from procarbazine, carmustine, lomustine, irinotecan, temozolomide, cisplatin, carboplatin, methotrexate, etoposide, cyclophosphamide, ifosfamide, and vincristine.

In another particular embodiment, the antiproliferative treatment defined hereinbefore may involve, in addition to the compound of the invention, conventional surgery or radiotherapy or chemotherapy, wherein the chemotherapy may include one or more chemotherapeutic agents selected from a BCL-2 family inhibitor (e.g. Venetoclax and/or navitoclax), a BTK inhibitor (e.g. Ibrutinib, Acalabrutinib, Tirabrutinib (ONO/GS-4059), BGB-3111 or Spebrutinib (CC-292) or a TNF inhibitor (e.g. Lenalidomide).

Such conjoint treatment may be achieved byway of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and another anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### Examples

The following examples are provided solely to illustrate the present invention and are not intended to limit the scope of the invention, as described herein.

### Abbreviations

Abd for antibody derived
AITL for angioimmunoblastic T cell lymphoma
ALL for acute lymphoblastic leukaemia
AR for androgen receptor
BL for Burkitt lymphoma
BME for 2-Mercaptoethanol
Boc for tert-butyloxycarbonyl
BRET for bioluminescence resonance energy transfer
CML for chronic myeloid leukaemia
cSPR for competitive surface plasmon resonance
DLBCL for diffuse large B cell lymphoma
DMF for N,N-dimethylformamide
DMSO for dimethylsulfoxide.
EDTA for ethylenediaminetetraacetic acid
ESI for electrospray ionisation
EtOAc for ethyl acetate
FBS for fetal bovine serum
FL for follicular lymphoma
GFP for green fluorescent protein
GNRH for gonadotropin-releasing hormone
GS for glycine serine
h for hours
HATU for N-[(dimethylamino)-1H-1,2,3-triazolo-[4,5-b]pyridin-1-ylmethylene]-N-methylmethanaminium hexafluorophosphate N-oxide
HBSS for Hank's balanced salt solution
HPLC for high performance liquid chromatography
HPLC for High Pressure Liquid Chromatography.
HRMS for high resolution mass spectrometry
HTS for high throughput screening
iDAbs for intracellular domain antibodies
IPTG for isopropyl 1-thio-beta-D-galactopyranosid
LB for lysogeny broth
LC-MS for liquid chromatography mass spectrometry
LH for leutenising hormone
LMO2 for LIM domain only protein
LRMS for low resolution mass spectrometry
mCPBA for meta-chloroperoxybenzoic acid
MI for Molecular Ion
Min for minutes
NMR for Nuclear Magnetic Resonance.
NSCLC for non-small cell lung cancer
PAL for photoaffinity labelling
PAMPA for parallel artificial membrane permeability assay
Papp for apparent permeability
PARP for poly ADP ribose polymerase
PBS for phosphate buffer saline
PCR for polymerase chain reaction
PEG for polyethylene glycol
PPI for protein protein interactions
ppm for parts per million
PS for penicillin/streptomycin
PVDF for polyvinylidene difluoride
RT for Room temperature
RU for response units
SAR for structure activity relationship
SCC for squamous cell carcinoma
SDS-PAGE for sodium dodecyl sulfate polyacrylamide gel electrophoresis
SERM for selective estrogen receptor degrader
SPR for surface plasmon resonance
TFA for trifluoroacetic acid
THF for tetrahydrofuran
TLC for thin layer chromatography

### FIGURES

FIG. 1: cSPR chemical library screen for LMO2 binding compounds

(A) The SPR streptavidin chips used for the cSPR screen were channel 1: blank; channel 2: LMO2-ΔLID; channel 3: KRAS; channel 4: LMO2-GS-iDAb. Competitive SPR screening of the PPI-NET compound library (1,500 compounds in total) was done at a single concentration of 150 μM for each compound (Cruz-Migoni et al., 2019). Response levels were normalised by subtracting the response measured against one of the two control reference proteins: LMO2-iDAb LMO2 fusion protein or KRAS and normalised response units (RUₙₒᵣₘ). Hit compounds Abd-L1, L2, L3 and L4 are indicated by orange dots. Both LMO2-iDAb and KRAS were used as negative reference proteins. (C) chemical structures and molecular weights (MW) of four hits are shown, together with PPI-NET plate locations (P number) and the designated Antibody-derived (Abd) number. Abd-L1 and Abd-L2 are homologues that differ only by the presence of 1H-pyrrolo[2,3-b]pyridine in Abd-L1 or pyrazolo[1,5-a]pyridine in Abd-L2, both indicated by the red oval. Note: additional amounts of Abd-L2 and Abd-L3 were not available commercially. (D) The LMO2-binding hit compound Abd-L1 was assessed in vitro with NMR waterLOGSY with binding to LMO2-LID or to LMO2-ΔLID comparing the spectra with the proton NMR of the compounds. (E) Caco-2 permeability assay that shows cell import and cell export data for Abd-L1. See also related FIG. 7.

FIG. 2: Establishing a BRET-based LMO2-iDAb biosensor amenable for a high throughput screening of small molecule libraries

The BRET2 assay comprises live in-cell generation of signal following interaction of a donor protein (in this case LMO2-RLuc8) and an acceptor protein (in this case GFP²-anti-LMO2 iDAb) and BRET signal (energy transfer from activated RLuc8 to GFP²). (A) BRET donor saturation assay with donor LMO2 and different mutant iDAb LMO2 acceptors, iDAb LMO2_{dm}, LMO2_{dm1-dm6}. (B) BRETₘₐₓ and BRET₅₀ values from the donor saturation curves displayed in A. (C) Western blot data for the expression of the GFP²-iDAb LMO2 and mutants (using anti-GFP antibody) and expression of LMO2-RLuc8 (with anti-LMO2 antibody). α-tubulin is the loading control. (D) BRET competition assay of LMO2-RLuc8 and the different GFP²-iDAb LMO2_{d}mx by expression of a non-relevant control iDAb (anti-RAS (Tanaka et al., 2007); Ctl, white bars) or unmutated iDAb LMO2 (black bars) as competitors. This competition is performed at the lowest dose of competitor (i.e. 0.1 μg). The percentage inhibition by iDAb LMO2 compared to iDAb Ctl is displayed. The iDAb LMO2_{dm3} mutant chose for the cell-based screening assay is coloured in blue. Each experiment was performed twice. Where error bars are presented (A, D), they correspond to mean values±standard deviation (SD) of biological repeats. See also related FIGS. 8-10.

FIG. 3: Cell-based high-throughput screening for inhibitors of LMO2-iDAb LMO2 PPI

(A) Scheme for the cell-based high throughput screening (HTS) where a diverse chemical library of 10,720 compounds was screened using a BRET cell assay to determine diminution of signal generated by interaction of LMO2-RLuc8 and GFP²-iDAb_{dm3}. (B) Scatter plot of the normalised BRET signal from 10,720 compounds tested at 10 μM. 34 compounds (primary hits) caused inhibition of BRET signal below a cut-off of 3 times the SD (minus, −3×SD) of the DMSO BRET signal (Lavoie et al., 2013). Some primary hits are pinpointed in orange. (C, D) Confirmation of inhibition of BRET signal from interaction LMO2 with iDAb LMO2_{dm3} (C) and for interaction of LMO2 with unmutated iDAb (D). Eight hits (depicted by blue bars) were confirmed to decrease LMO2-iDAb LMO2_{dm3} signal by at least 3×SD of the BRET signal with DMSO control (i.e. DMSO BRET signal±3×SD: 12.2±3.6, threshold set at 8.6 and shown with the dotted line) without affecting LMO2-iDAb LMO2 interaction (i.e. DMSO BRET signal±3×SD: 30.3±3.4, threshold set at 26.9 and shown with the dotted line). P24H7 compound highlighted with a red star is an example of compound that was not pursued further as it affects both iDAb LMO2_{dm3} and iDAb LMO2 interaction with LMO2. Experiments in C, D were performed twice. Error bars presented in C, D correspond to mean values±SD of biological repeats. See also related FIG. 11.

FIG. 4: Structure-activity relationship of antibody derived (Abd) LMO2-binding compounds

The chemical structures of the hit matter from the BRET screen were examined and a family of compounds identified. (A) Chemical structures of the 8 re-synthesised hits (Abd-L5 to Abd-L12) with their respective molecular weight (MW). (B) Dose response inhibition of LMO2-iDAb LMO2_{dm3} interaction by compounds Abd-L5 to Abd-L12 (concentration range: 1, 10, 20 M). The data were obtained from duplicate biological experiments. Error bars correspond to mean values±SD of biological repeats. (C) SAR study of Abd-L9 compound as template. The compound was divided into four substituents (named A-D) that were substituted by various other chemical groups to give new compounds. (D) Structures of representative compounds Abd-L15-L23. The new compounds were tested by BRET assays with LMO2-iDAb LMO2_{dm3} interaction. The different SAR-derived compounds are shown with their MW and the percentage of BRET inhibition of the interaction LMO2-iDAb LMO2_{dm3} at 20 μM. See also related FIGS. 12 & 13.

FIG. 5: Abd compounds bind to LMO2 in vitro

The in vitro binding properties of Abd-L compounds confirmed by waterLOGSY NMR and by photoaffinity labelling. (A-C) WaterLOGSY NMR was carried out to determine Abd-L9 (A), Abd-L10 (B) and Abd-L13 (C) binding to LMO2 fused to the LID of LDB1 (LMO2-LID) or to a shortened version of LID in LMO2-ΔLID. Each of these compounds bind to LMO2-ΔLID (green) and not to LMO2-LID (purple) protein. (D) Chemical structure of Abd-L26 designed for photoaffinity labelling (PAL) to LMO2 protein. This is a compound built on Abd-L15/16 template, with a benzophenone photoreactive moiety, a linker and a biotin tag. (E-G) Pulldown of scFv-LMO2 recombinant protein by Abd-L26 with avidin beads, treated or not with UV light. The protein was either incubated alone (lane 1) or with 20 μM of Abd-L26 (lane 2) without UV treatment. The protein was also incubated with 20 μM Abd-L26 alone (lane 3) or with 100 μM Abd-L9 as competitor (lane 4) treated by UV light. The beads were washed and a Western blot showed the quantity of crosslinked LMO2 on the beads with an anti-biotin (E), anti-LMO2 (F) and an anti-HIS antibody (G).

FIG. 6: Evaluation of LMO2-binding Abd compound potency in cells

The potency and specificity of the LMO2 Abd compounds was evaluated in dose response BRET assays (A-C) In BRET assays, Abd-L9, Abd-L10, Abd-L16 and Abd-L22 compounds were assessed in dose inhibition responses for (A) LMO2-iDAb LMO2_{dm3}, (B) LMO2-LDB1 and (C) LMO2-iDAb LMO2 (unmutated iDAb) BRET interactions. (D) Dose response assays of Abd-L15, Abd-L17, Abd-L18 and Abd-L19 with LMO2-iDAb LMO2_{dm3}, LMO2-LDB1 and LMO2-iDAb LMO2 BRET interactions. Each experiment was performed twice. Error bars presented in A-D correspond to mean values±SD of biological repeats. See also related FIG. 14.

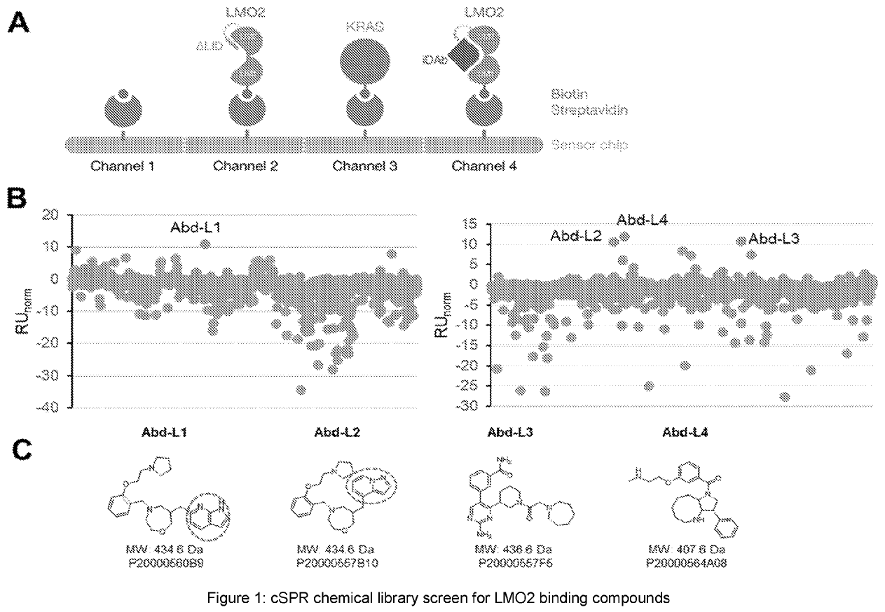

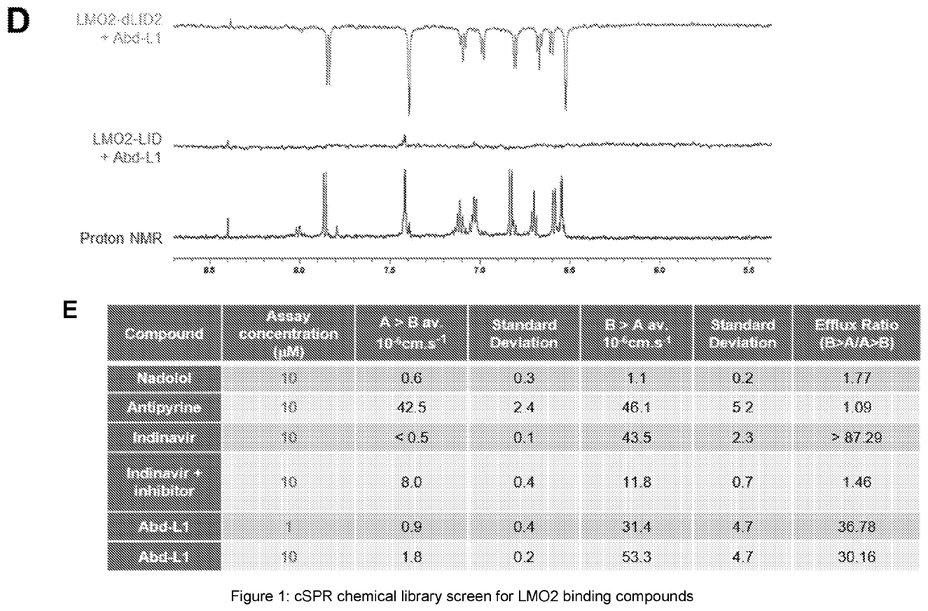

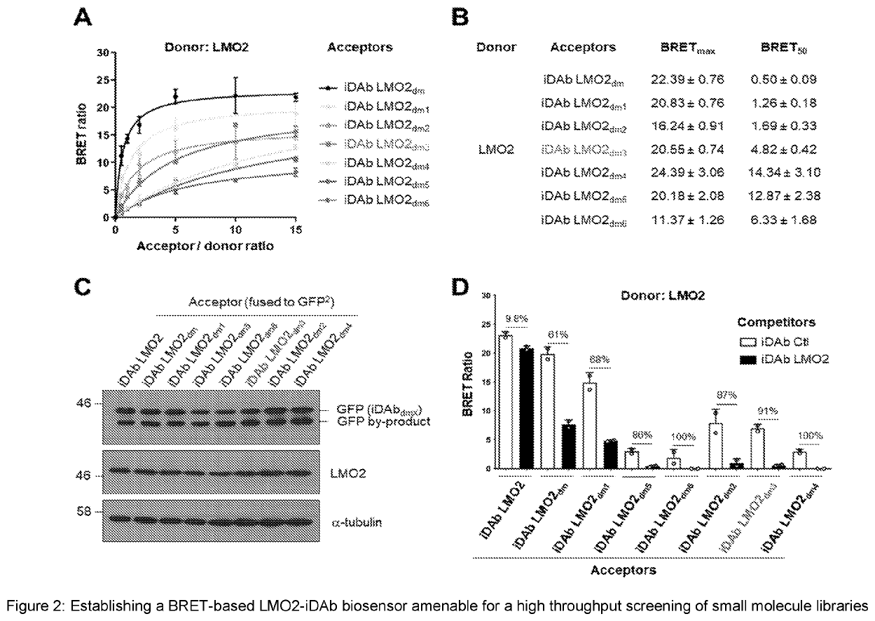

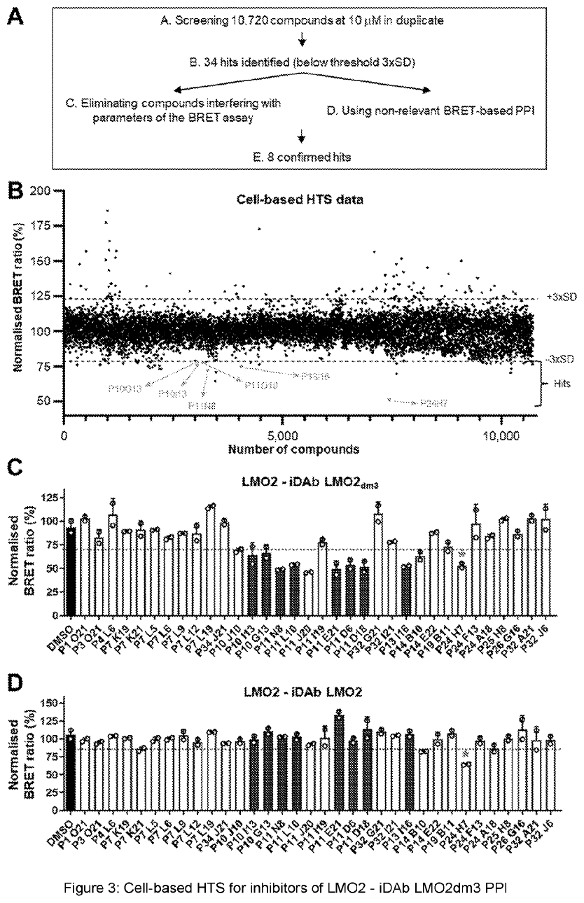

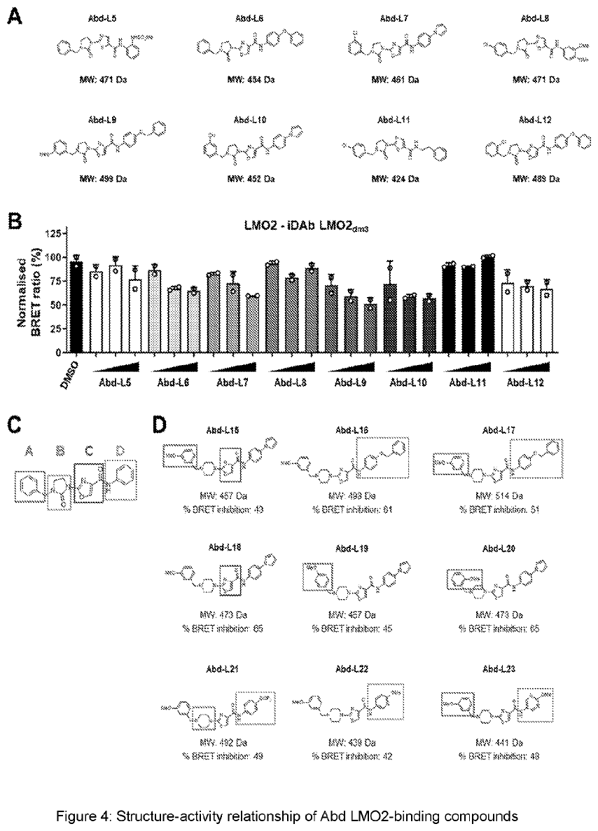

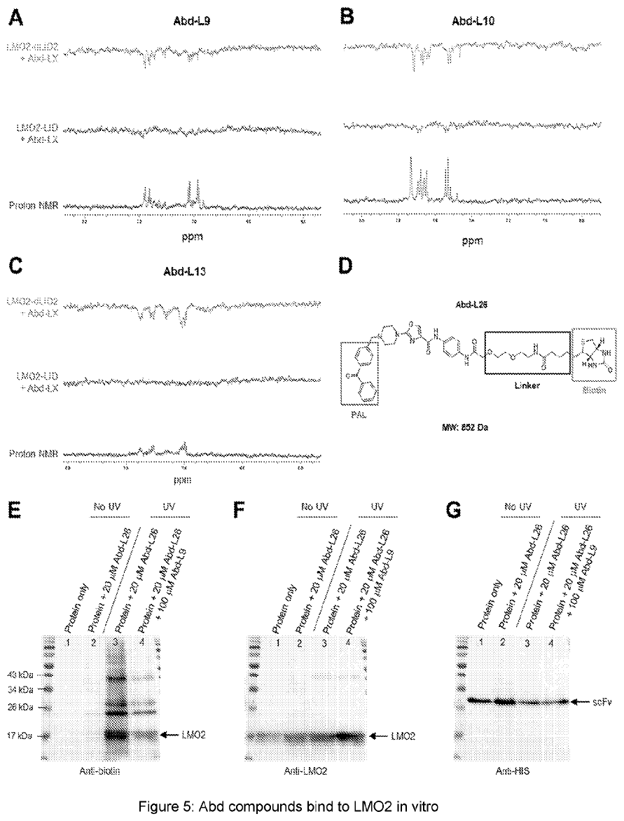

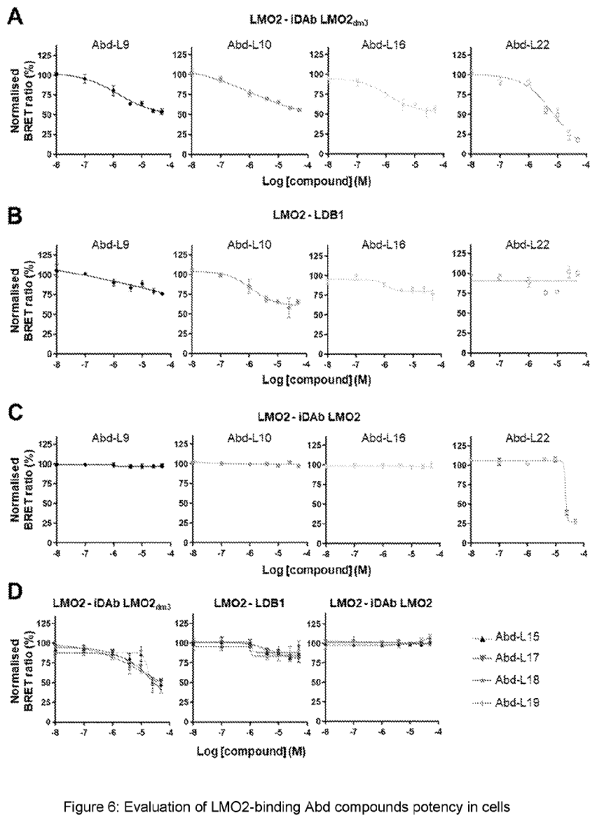

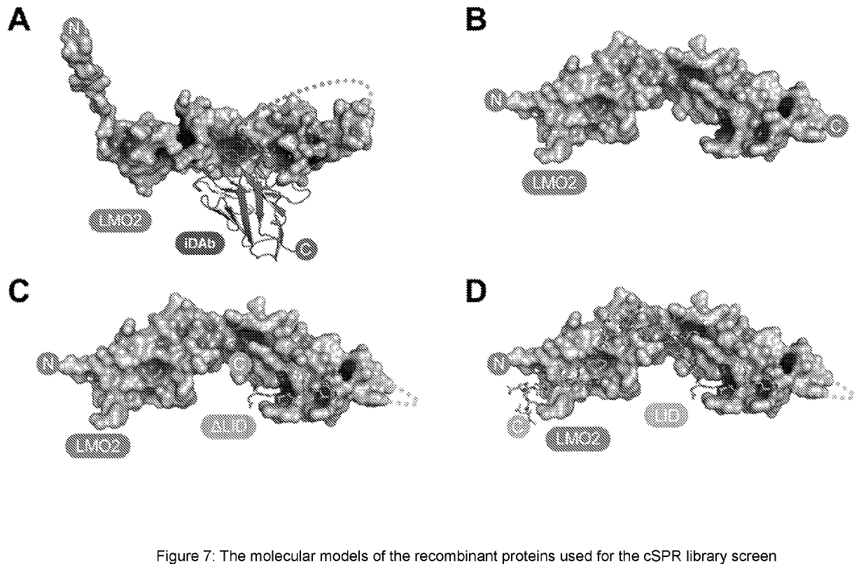

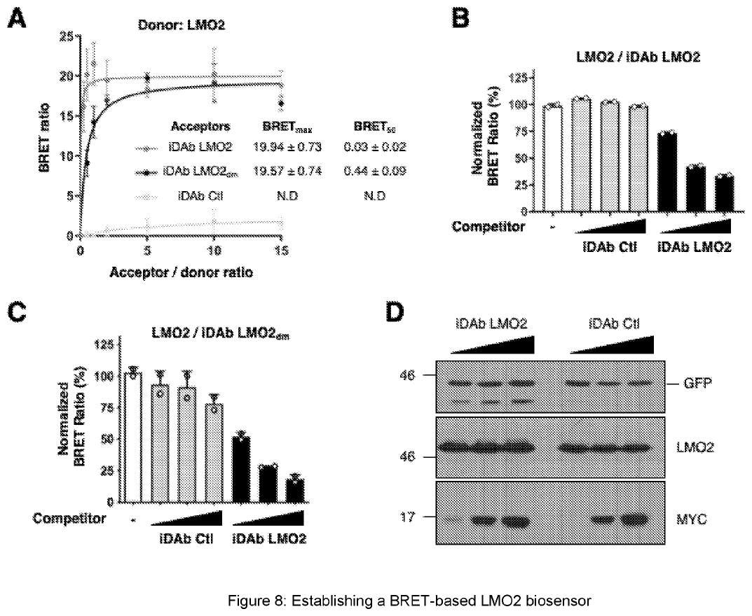

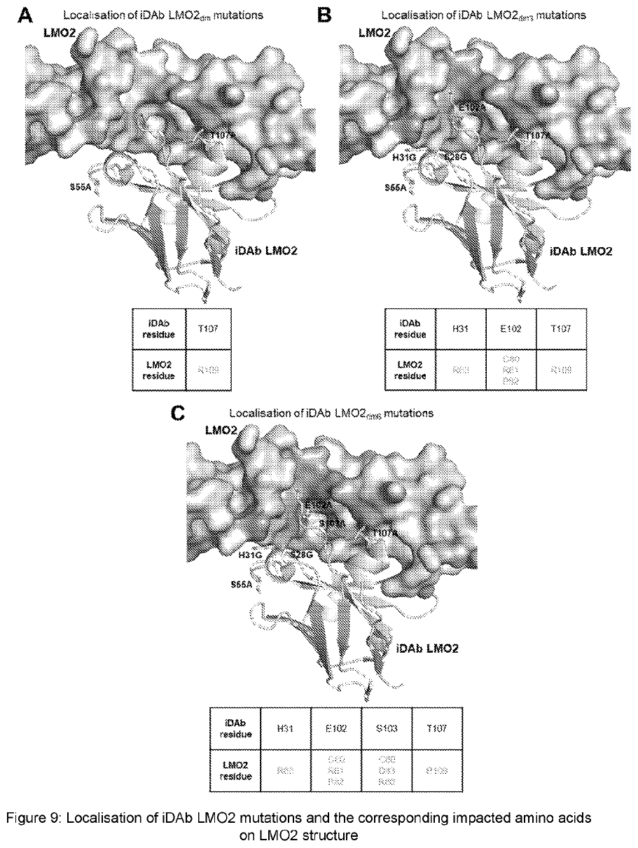

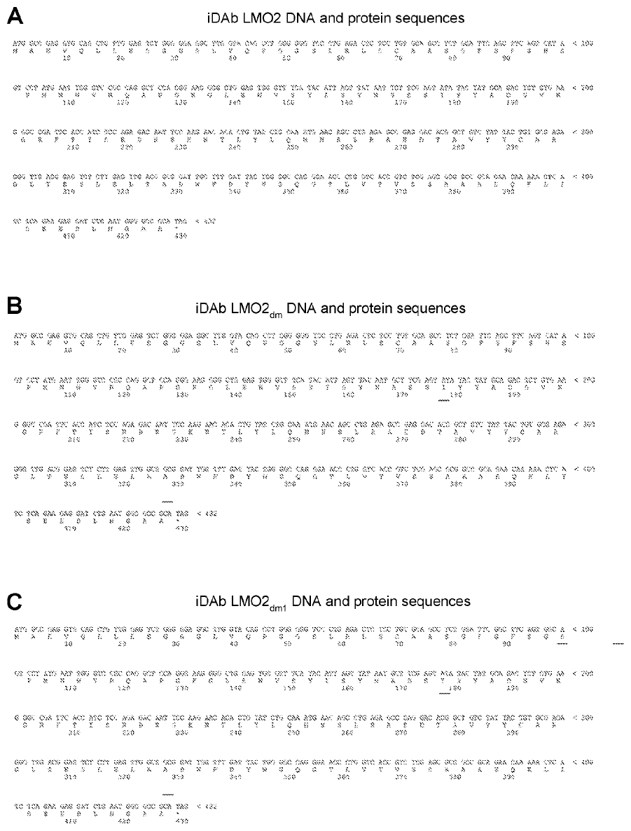

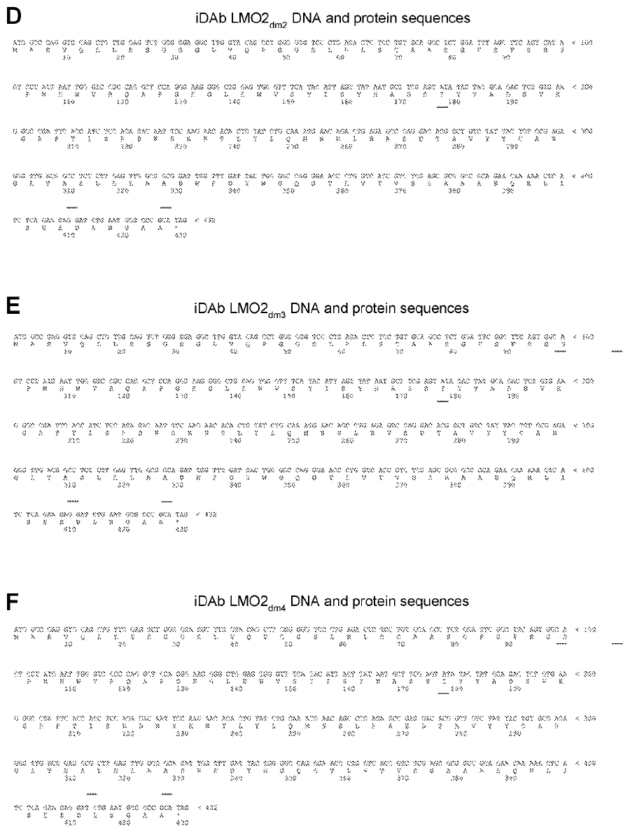

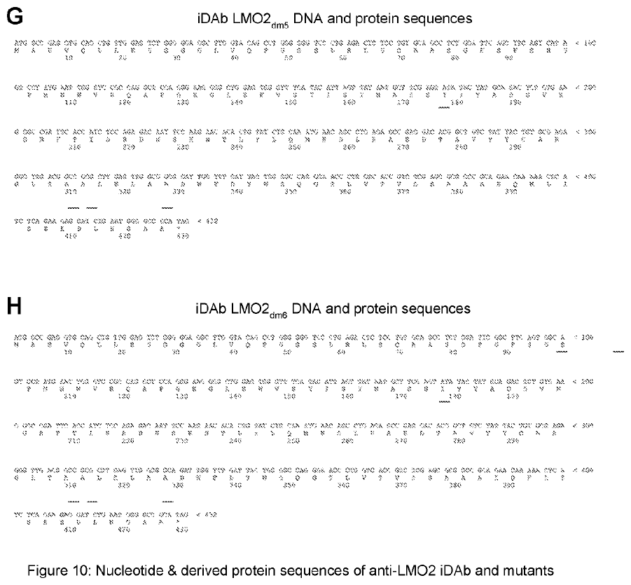

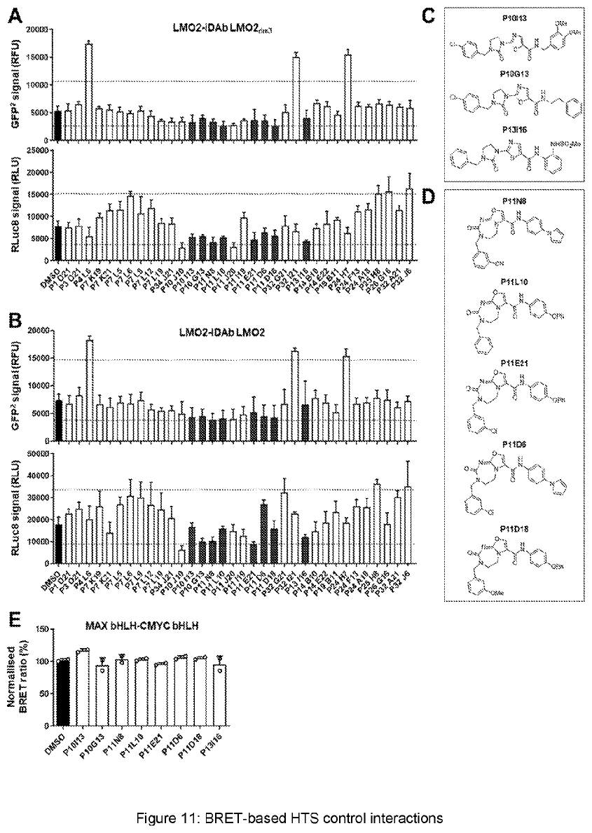

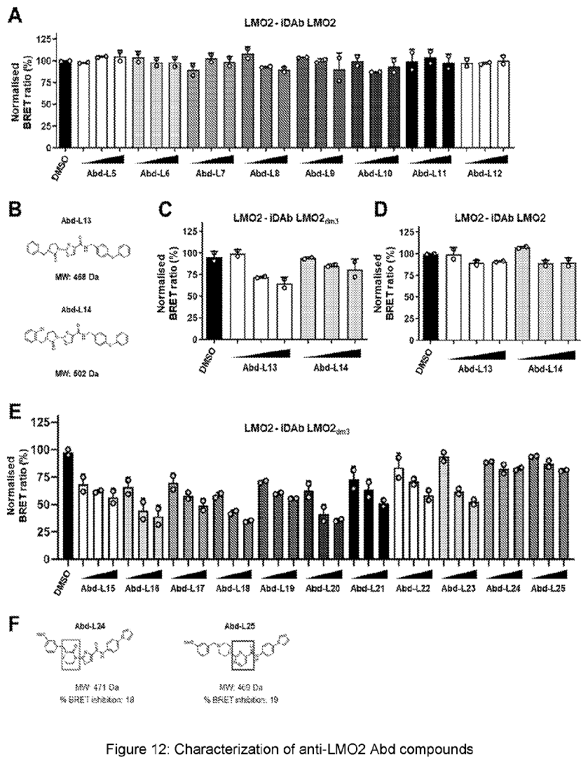

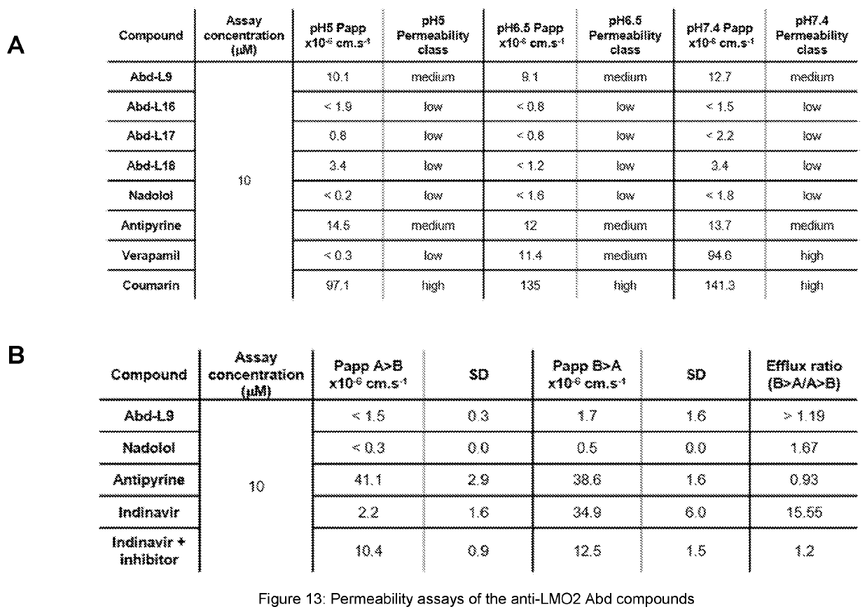

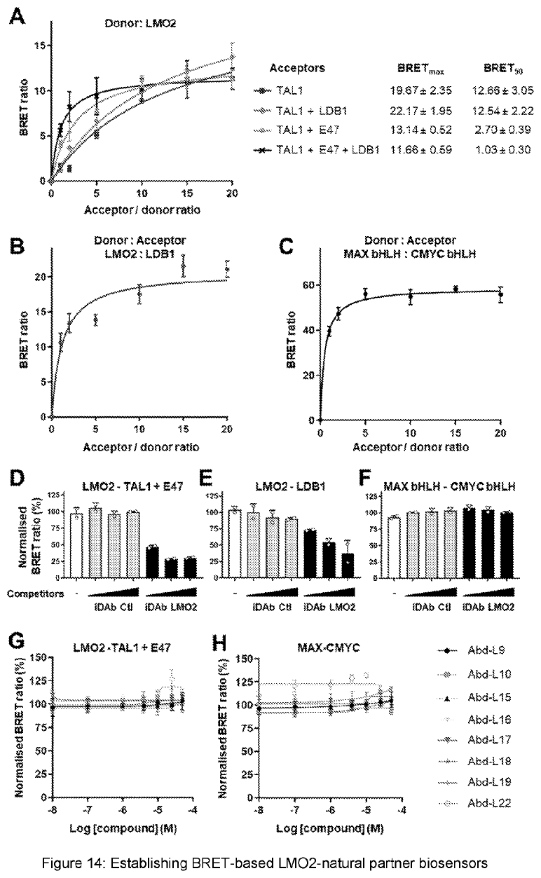

FIG. 7 related to FIG. 1: Molecular models of the recombinant proteins used for the cSPR library screen

(A) LMO2-iDAb LMO2 (PDB ID: 4KFZ), (B) LMO2 only, (C) LMO2-ΔLID and (D) LMO2-LDB1 LID domain fusion protein. LMO2 is shown as a surface representation, the LDB1 LID domain as sticks and iDAb LMO2 in ribbon context with a dotted line representing the linker between LMO2 and iDAb. The LMO2 only, LMO2-LID and LMO2-ΔLID structures are based on the LMO2-LDB1 crystal structure (PDB ID: 2XJY). The N- and C-termini of the proteins are marked for orientation purposes.

FIG. 8 related to FIG. 2: Establishing a BRET-based LMO2 biosensor

(A) BRET donor saturation assay with LMO2-RLuc8 as donor and GFP²-iDAb LMO2, GFP²-iDAb LMO2_{dm} and GFP²-iDAb Ctl (non-relevant anti-RAS iDAb) as acceptors. (B) BRET competition assays between the LMO2-iDAb LMO2 interaction when either iDAb Ctl (grey bars) or iDAb LMO2 (black bars) were used as competitors (the control no competitor (−) is the white bar). (C) BRET competition assay between LMO2-iDAb LMO2_{dm} interaction and the same competitors as panel B. (D) Western blot analysis of proteins from the BRET competition assay cells shown in panel C. Anti-GFP antibody shows iDAb LMO2_{dm} expression, anti-LMO2 expression of LMO2-RLuc8 and anti-CMYC antibody shows expression of the competitors. Each experiment was performed twice. When error bars are presented, they correspond to mean values±SD of biological repeats.

FIG. 9 related to FIG. 2: Localisation of iDAb LMO2 mutations and the corresponding impacted amino acids on LMO2 structure

The part of LMO2 around the hinge region is shown in grey and in each panel, relevant amino-acids interacting with the iDAb LMO2 are shown in red. (A) Localisation of iDAb LMO2_{dm} mutations (S55A and T107A) are shown in yellow on the parental iDAb LMO2 structure (cyan) with the affected, interacting LMO2 residue (R109) shown in red on LMO2 structure. (B) Localisation of iDAb LMO2_{dm3} mutations (S28G, H31G, S55A, E102A and T107A) are shown in yellow on the parental iDAb LMO2 structure with the affected LMO2 residues shown in red on LMO2 structure. (C) Localisation of iDAb LMO2_{dm6} mutations (S28G, H31G, S55A, E102A, S103A and T107A) are shown in yellow on the parental iDAb LMO2 structure with the affected LMO2 residue shown in red on LMO2 structure. LMO2-iDAb LMO2 structure used is PDB 4KFZ. Each panel has a table listing the interacting amino acids of LMO2 and iDAb.

FIG. 10 related to FIG. 2: DNA and proteins sequences of iDAb LMO2 and its mutants

(A) DNA and protein sequences of iDAb LMO2. (B-H) DNA and protein sequences of each iDAb LMO2_{dm1}-LMO2_{dm6}. The mutated amino acids compared to the parental iDAb LMO2 underlined in brown.

FIG. 11 related to FIG. 3: BRET-based HTS control interactions

(A) GFP²-only and RLuc8-only signal controls from FIG. 3C. (B) GFP²-only and RLuc8-only signal controls from the BRET experiment displayed on FIG. 3D. Compounds modifying RLuc8 luminescence or intrinsic GFP² fluorescence by greater than two-fold were discarded (delimitated with the dotted lines calculated to the DMSO controls) (Lavoie et al., 2013). (C, D) Chemical structures of the 8 confirmed hits from the HTS that are divided into two related subfamilies, the 5 membered ring (C) and 7 membered ring (D). (E) BRET competition assay between the 8 hits and the non-relevant PPI MAX bHLH-CMYC bHLH. Each experiment in (A, B, E) was performed twice. The error bars correspond to mean values±SD of biological repeats.

FIG. 12 related to FIG. 4: Characterisation of anti-LMO2 Abd compounds

(A) Dose response effect of the Abd-L5 to Abd-L12 compounds on LMO2-iDAb LMO2 (unmutated) interaction (Abd concentration used: 1, 10, 20 μM). (B) Chemical structures, and their respective molecular weights (MW), of Abd-L13 and Abd-L14, which are two analogues of Abd-L8 and Abd-L21 respectively. (C) Abd-L13 and Abd-L14 were tested by BRET assays with LMO2-iDAb LMO2_{dm3} or (D) LMO2-iDAb LMO2 interactions (Abd concentration used: 1, 10, 20 μM). (E) Dose response effect of the Abd-L15 to Abd-L25 compounds on LMO2-iDAb LMO2_{dm3} interaction (Abd concentration used: 5, 10, 20 μM). (F) Chemical structures of Abd-L24 and Abd-L25 with their respective molecular weights (MW) and their percentage of BRET inhibition of the interaction LMO2-iDAb LMO2_{dm3} at 20 μM. These are two analogues, modified on their positions B and C respectively, which do not affect the BRET interaction LMO2-iDAb LMO2_{dm3}. Experiments in A, C, D, E were performed twice. Error bars presented in A, C, D, E correspond to mean values±SD of biological repeats.

FIG. 13 related to FIG. 4: Permeability assays of the anti-LMO2 Abd compounds (A) Parallel artificial membrane permeability assay (PAMPA) with Abd-L9, Abd-L16-18. (B) CaCo-2 permeability assay with Abd-L9 compound.

FIG. 14 related to FIG. 6: Establishing BRET-based LMO2-natural partner biosensors

(A) BRET donor saturation assay with RLuc8-LMO2 as donor and full-length TAL1-GFP² as acceptor with or without LDB1 and/or E47 co-expression. BRETₘₐₓ and BRET₅₀ values are indicated for each BRET pair. (B) BRET donor saturation assay between LMO2-RLuc8 (donor) and full-length GFP²-LDB1 (acceptor). (C) BRET donor saturation assay with MAX bHLH-RLuc8 as donor and CMYC bHLH-GFP² as acceptor. (D-F) BRET competition assays using iDAb Ctl (grey bars), iDAb LMO2 (black bars) as competitors or no competitor (-, white bar) between the following interactions: (D) LMO2-TAL1+E47, (E) LMO2-LDB1 and (F) MAX bHLH-CMYC bHLH. (G, H) BRET dose response assays with LMO2-TAL1+E47 interaction (G) and MAX bHLH-CMYC bHLH interaction (H) with the indicated Abd-L compounds. Each experiment was performed twice. The error bars correspond to mean values±SD of biological repeats.

### ANALYTICAL METHODS

### Cell Culture

HEK293T cells were grown in DMEM medium (Life Technologies) and supplemented with 10% FBS (Sigma) and 1% Penicillin/Streptomycin (PS) (Life Technologies). Cells were grown at 37° C. with 5% CO₂.

### Molecular Cloning

The DNA sequences encoding for LMO2-ΔLID (UniProt P25791; residues 26-156 and UniProt Q86U70; residues 334-344, joined by a GS linker), and LMO2-iDAb were cloned into the expression vector pOPINS via the restriction sites KpnI and HindIII, incorporating an AviTag into the 5′ primer. The vector encodes an N-terminal hexa-histidine tag and SUMO tag. The final protein expression constructs encoded for a single fusion protein consisting of His-SUMO-Avi-LMO2-GS-ΔLID and His-SUMO-Avi-LMO2-GS-iDAb. A construct encoding KRAS^{G12V}₁₆₆ with N-terminal His tag and TEV protease cleavage site was modified via PCR to include an AviTag between the TEV site and protein-coding sequence. For the SPR screening, all proteins were prepared with biotin tags.

iDAb LMO2 mutations were generated by PCR site-directed mutagenesis using pEF-GFP²-iDAb LMO2_{dm} as template (Bery et al., 2018) (i.e. iDAb LMO2 S55A/T107A). The following mutations were introduced: iDAb LMO2 S28G/H31G/S55A/T107A, iDAb LMO2 S55A/E102A/T107A, iDAb LMO2 S55A/E102A/S103A/T107A, iDAb LMO2 S28G/H31G/S55A/E102A/T107A, iDAb LMO2 S28G/H31G/S55A/S103A/T107A and iDAb LMO2 S28G/H31G/S55A/E102A/S103A/T107A (FIG. 10).

LMO2 cDNA was cloned into the pEF-RLuc8-MCS and pEF-MCS-RLuc8 plasmids, MAX bHLH (amino acids 37-102) was inserted into pEF-MCS-RLuc8 plasmid. iDAb LMO2, mutants iDAb LMO2 and full-length LDB1 were cloned into pEF-GFP²-MCS plasmid and full-length TAL1 and cMYC bHLH (amino acids 354-439) into pEF-MCS-GFP² plasmid.

cSPR Screening of PPI-NET Compound Library

As described previously (Cruz-Migoni et al., 2019) biotinylated LMO2-ΔLID, KRAS and LMO2-iDAb fusion were immobilized on a streptavidin-coated sensor chip SA (GE Healthcare) and the PPI-Net library compounds (comprising 1,500 compounds) were injected over the surface at 150 μM concentration. SPR experiments were carried out, as described, using a Biacore T200 (GE Healthcare) at 10° C. to preserve the protein immobilised on the sensor surface. Control proteins KRAS^{G12V}₁₆₆ and LMO2-iDAb were immobilised at ˜4000 RU and ˜6000 RU respectively to give approximately equimolar immobilisation levels of all three proteins on the sensor surface. Flow cell 1 was blocked by injecting 10 mM biocytin over the surface for 5 minutes at 10 μL·min⁻¹ and used as the reference channel. Immobilisation was carried out in HEPES running buffer (10 mM HEPES pH 7.4, 150 mM NaCl, 0.005% P20, 5 mM MgCl₂, 10 μM ZnCl₂). Compound solutions were prepared by transferring 1.5 μL PPI-Net stock compounds at 10 mM in 100% DMSO into 96-well plates (Greiner) using a multichannel pipette. 98.5 μL running buffer with 3.5% DMSO was added to yield a solution of 150 μM compound in running buffer with 5% DMSO. Compound solutions were injected over all 4 flow channels for 30 seconds at 30 L·min⁻¹ and dissociation monitored for 60 seconds. A negative control of running buffer with 5% DMSO was run after every 24 cycles. Data were referenced, solvent corrected and processed using the T200 evaluation software. Data were baseline-corrected using the negative control binding levels as a reference and binding levels measured against LMO2-ΔLID plotted against binding levels measured against the control proteins. Replacement Abd-L1 (PPI-NET identifier P20000560B9) and Abd-L4 (PPI-NET identifier P20000557F5) were purchased from Asinex. Their molecular weights were confirmed by mass spec: Abd-L1 LRMS m/z (ESI⁺) 435 [M+H]⁺; Abd-L2 LRMS m/z (ESI⁺) 437 [M+H]⁺ recorded on an Agilent 6120 spectrometer using solutions of MeOH.

### BRET2 Titration Curves and Competition Assays

For all BRET experiments (titration curves and competition assays) 650,000 HEK293T were seeded in each well of a 6 well plates. After 24 hours at 37° C., cells were transfected with a total of 1.6 μg of DNA mix, containing the donor+acceptor±competitor plasmids, using Lipofectamine 2000 transfection reagent (Thermo-Fisher). In dose response competition experiments, competitors were transfected with the following amount of DNA: 0.1; 0.5 and 1 μg. In single dose competition experiments, competitors were transfected with 0.1 g of DNA. Cells were detached 24 hours later, washed with PBS and seeded in a white 96-well plate (clear bottom, PerkinElmer, Cat #6005181) in OptiMEM no phenol red medium complemented with 4% FBS and cells were incubated for an additional 20-24 hours at 37° C. before the BRET assay reading. A detailed BRET protocol is provided by Bery and Rabbitts, (2019).

### Cell Treatment

Compounds were prepared in 100% DMSO at 10 mM. For BRET competition assays, cells were treated with the indicated compounds at concentration of 1 (or 5), 10 and 20 μM for 22 h. For BRET-based dose response experiments, cells were treated with compounds at concentration of 0.01, 0.1, 1, 4, 10, 25 and 50 μM for 22 h. The compounds were diluted in the BRET medium: OptiMEM no phenol red (Life Technologies) supplemented with 4% FBS and with a final concentration of 0.2% DMSO.

### BRET2 Measurements

BRET2 signal was determined immediately after injection of coelenterazine 400a substrate (10 μM final) to cells (Cayman Chemicals), using a CLARIOstar instrument (BMG Labtech) with a luminescence module. Total GFP² fluorescence was detected with excitation and emission peaks set at 405 nm and 515 nm respectively. Total RLuc8 luminescence was measured with the Luminescence 400-700 nm-wavelength filter.

The BRET signal or BRET ratio corresponds to the light emitted by the GFP² acceptor constructs (515 nm±30) upon addition of coelenterazine 400a divided by the light emitted by the RLuc8 donor constructs (410 nm±80). The background signal is subtracted from that BRET ratio using the donor-only negative control where only the RLuc8 fusion plasmid is transfected into the cells. The normalized BRET ratio is the BRET ratio normalized to a negative control (iDAb control or DMSO control) during a competition assay. Total GFP² and RLuc8 signals were used to control the protein expression from each plasmid.

### Western Blot Analysis

Cells were washed once with PBS and lysed in SDS-Tris buffer (1% SDS, 10 mM Tris-HCl pH 7.4) supplemented with protease inhibitors (Sigma) and phosphatase inhibitors (Thermo-Fisher). Cell lysates were sonicated with a Branson Sonifier and the protein concentrations determined by using the Pierce BCA protein assay kit (Thermo-Fisher). Equal amounts of protein (20 μg) were resolved on 12.5% SDS-PAGE and subsequently transferred onto a PVDF membrane (GE). The membrane was blocked with 10% non-fat milk (Sigma) in TBS-0.1% Tween20 and incubated overnight with primary antibody at 4° C. After washing the membrane was incubated with HRP conjugated secondary antibody for 1 hour at room temperature (RT, 22° C.). The membrane was washed with TBS-0.1% Tween and developed using Clarity Western ECL Substrate (Bio-Rad) and CL-XPosure films (Thermo-Fisher) or the ChemiDoc XRS+ imaging system (Bio-Rad). Primary antibodies include anti-LMO2 (1/1000, R&D System, Cat #AF2726), anti-GFP (1/500, Santa Cruz Biotechnologies, Cat #sc-9996), anti-biotin (1/1000, CST, Cat #5597S), anti-β-actin (1/5000, Sigma, Cat #A1978) and anti-αtubulin (1/2000, Abcam, Cat #ab4074). Secondary antibodies include anti-CMYC HRP-linked (Novus Biologicals, Cat #NB600-341), anti-mouse IgG HRP-linked (CST), anti-rabbit IgG HRP-linked (CST) and anti-goat IgG HRP-linked (Santa Cruz Biotechnologies).

High Throughput Chemical Screening with LMO2/iDAb LMO2 Mutant BRET Biosensor

The screen was carried out in 384-well plate format. An in-house library of 10,720 compounds (comprising 6991 compounds from BioFocus and 3729 from ChemBridge) were in 96-well plate format. The library was compressed into 384-well plate format for the HTS purpose. The volume and quantities indicated are for 40 assay 384-well plates. The screen was carried out in duplicate at 10 μM. Two sessions of HTS, containing 5,360 compounds each, were screened in 68 assay plates (34 assay plates in duplicate).

Before starting, HEK293T cells were seeded into 2×T175 flask. Three days later, the 2×T175 were split into 6×T175.
Day 1: cell seeding: Cells were harvested from 6×T175 flasks at ˜70% confluency. The cells were resuspended in 110 mL of complete DMEM and 120×10⁶ inoculated into each of two Corning HYPERFlask M cell culture vessels (Corning, Cat #10030) and 560 mL of medium was added to fill one HYPERFlask.
Day 2: cell transfection with pEF-LMO2-RLuc8 and pEF-GFP²-iDAb LMO2_{dm3}: For each HYPERFlask 10 mL of OptiMEM was added together with 19 μg of pEF-LMO2-RLuc8, 37 g of pEF-GFP²-iDAb LMO2_{dm3} and 244 μg of pEF-empty-cyto-myc plasmids. 750 μL of Lipofectamine 2000 was added in 10 mL of OptiMEM, mixed gently. The 10 mL of DNA dilution was added and incubated for 20 minutes. The DNA/Lipofectamine 2000 mix was added in 500 mL of complete DMEM and the medium of the HYPERFlask had been removed. Finally, the medium+transfection mix was carefully poured into the HYPERFlask without creating any bubbles and the flask was filled with medium.
Day 3: cell seeding in 384-well plates: The cells were harvested with 100 mL of trypsin that were added per HYPERFlask, incubated for 2 minutes at 37° C. and the trypsinized cells transferred to a beaker containing 100 mL of complete DMEM. Each flask was washed once with 100 mL of complete DMEM, mixed gently but thoroughly to ensure single cell suspensions (final volume for one flask: 300 mL). 90×10⁵ cells were added per 250 mL Corning centrifuge tube (4×250 mL centrifuge tubes were used which was 360×10⁶ transfected cells in total) and the cells were centrifuged at 220×g for 5 minutes at RT. Each cell pellet (4 in total) was gently resuspended in 200 mL of Opti-MEM without red phenol+4% FBS+1% PS (hereafter called BRET medium) to a final concentration of 0.45×10⁶ of cells/mL. The cells were seeded in white 384-well plates (clear bottom, PerkinElmer, Cat #6007480) with a PerkinElmer Janus liquid handling workstation housed in a Category 2 enclosure (45 μL/well; 20 000 cells). A blank plate was first used to remove any air bubble in the liquid handling workstation.
Day 3: library dilution: 100 μM stock solutions were prepared for each compound in the library (the initial concentration of the library was 10 mM). 150 nL of 10 mM of each compound was added with an Echo Acoustic Dispenser (Labcyte) into 15 μL of BRET medium, giving a final concentration of 100 μM.
Day 3: compounds addition: 1% DMSO was prepared in BRET medium. 5 μL of 1% DMSO solution was dispensed in the columns 1, 2 and 23, 24 as negative controls. Compounds were added to cells in 5 μL (100 μM) in each well using the Perkin Elmer Janus liquid handling workstation (10 μM final concentration, 0.1% DMSO) and the plates Incubated for 20 hours.
Day 4: plate reading: A PHERAstar FSX plate reader (BMG Labtech) was used to read the plates equipped with a BRET2 optic module. The GFP² signal of each plate was first measured to assess the relative cell number in each well. After the GFP² reading, the bottom of each plate was covered with a white tape. 80 mL of 100 μM BRET substrate (i.e. coelenterazine 400a, Cayman Chemicals) was prepared by dissolving 3 mg of coelenterazine 400a in 32 mL of 100% ethanol and the volume brought to 80 mL by adding 48 mL of BRET medium. BRET reading was carried out by adding 5.5 μL of coelenterazine 400a (final concentration of 10 μM) using injectors and reading the BRET signal of each well. The reading time for one 384-well plate was about 8 minutes, therefore ˜4.5 hours to read 34 plates.

### Recombinant Protein Expression

LMO2-LID protein was expressed in BL21(DE3)-Rosetta2 pLysS cells (Novagen) and HIS-SUMO-AVI-LMO2-ΔLID in Lemo21(DE3) cells (NEB, Cat #2528J). Bacterial cells were cultured at 37° C. in LB medium supplemented with 50 μg·mL⁻¹ kanamycin and 32 μg·mL⁻¹ chloramphenicol. At mid-log phase, expression was induced by addition of IPTG to a final concentration of 0.5 mM and supplemented with 0.1 mM ZnCl₂. The cultures were incubated overnight at 18° C. with shaking at 220 rpm. Cells were harvested by centrifugation and resuspended in binding buffer (50 mM HEPES, pH 7.4, 500 mM NaCl, 5% glycerol and 5 mM imidazole). Resuspended pellets were stored at −20° C. Thawed cell pellets were supplemented with complete EDTA-free protease inhibitors at 1× concentration (Roche), 5 μL DNAse per liter of culture volume and MgSO₄ to a final concentration of 2 mM. Cell suspensions were stirred on ice for 15 minutes and lysed using a Constant Systems Cell Disruptor at 23 kpsi, 4° C. Cell extracts were clarified by centrifugation. His-tagged proteins were purified under gravity flow using nickel-Sepharose (GE Healthcare) columns. Bound proteins were washed with 2×50 mL of binding buffer and then 25 mL of wash buffer (50 mM HEPES, pH 7.4, 500 mM NaCl, 5% glycerol and 20 mM imidazole). Bound proteins were eluted with 5 mL of elution buffer (50 mM HEPES, pH 7.4, 500 mM NaCl, 5% glycerol and 50 mM imidazole) and 2×5 mL elution buffer 2 (50 mM HEPES, pH 7.4, 500 mM NaCl, 5% glycerol and 500 mM imidazole). SUMO tags were removed by incubating with SUMO protease overnight at 4° C. Proteins were further purified on a HiLoad 16/60 Superdex 200 Prep Grade column using an ÅKTA Avant system (GE Healthcare) buffered in 10 mM HEPES pH 7.4, 250 mM NaCl. For proteins to be used in SPR experiments, N-terminal AviTags were biotinylated by incubating with BirA enzyme overnight at 4° C. in the presence of 20 mM MgCl₂, 500 μM biotin and 2 mM ATP. Proteins were further purified on a HiLoad 16/60 Superdex 200 Prep Grade column using an ÅKTA Avant system (GE Healthcare) buffered in 10 mM HEPES pH 7.4, 250 mM NaCl and 0.5 mM DTT. KRAS₁₆₆^{G12V} was purified as previously described (Cruz-Migoni et al., 2019), with the addition of the BirA incubation step for biotinylation of the purified protein.

Purification of scFv-LMO2 for PAL Analysis

For co-expression of recombinant LMO2 and anti-LMO2 scFv, the scFv was cloned into an existing bicistronic expression vector (pRK-His-TEV-VH576-LMO2, Sewell et al 2014). DNA encoding the scFv was amplified by PCR and cloned into the pRK vector to replace the VH576 using NcoI and EcoRI restriction sites. Plasmid DNA was transformed into E. coli C41 (DE3) cells for protein co-expression. A single colony was used to inoculate 50 mL of LB media containing 100 μg·mL⁻¹ ampicillin which was grown overnight at 37° C., shaking at 225 rpm. The overnight seed culture was diluted 1:100 in 8×1 L of LB containing 100 μg·mL⁻¹ ampicillin. The cultures were grown at 37° C., shaking at 225 rpm until an OD₆₀₀ of 0.6 was reached. ZnSO₄ was added prior to induction to a final concentration of 0.1 mM. Protein expression was induced by the addition of 0.5 mM IPTG (isopropyl 1-thio-beta-D-galactopyranosid) and the cells were incubated overnight at 16° C., shaking at 225 rpm. Cells were harvested by centrifugation at 6000 rpm, for 20 minutes at 4° C. Cell pellets were resuspended in lysis buffer (20 mM Tris pH 8.0, 250 mM NaCl, 20 mM imidazole, 0.1 mM ZnSO₄, 5 mM 2-mercaptoethanol, 5% glycerol) containing EDTA-free protease inhibitor cocktail tablets (Roche, Germany) prior to lysis at 25 kPSI, 4° C. using a cell disruptor system (Constant Systems Ltd, UK). The cell lysate was incubated with DNase I and 2 mM MgCl₂ for 20 minutes at RT before being clarified by centrifugation at 22,000 rpm for one hour at 4° C. LMO2 and anti-LMO2 scFv were co-purified using a 5 mL HisTrap HP column (GE Healthcare, UK) using a 50 mL imidazole gradient from 20 mM to 300 mM. The protein was concentrated to 1.5 mL and purified further by gel filtration using a HiLoad 16/600 Superdex 75 column (GE Healthcare, UK) in 20 mM Tris pH 8.0, 250 mM NaCl, 1 mM DTT. The co-purification of LMO2 and anti-LM02 scFv was verified by standard Western blotting using anti-LMO2 (R&D Systems, AF2726) and anti-His-HRP (Sigma, A7058).

### WaterLOGSY NMR

WaterLOGSY NMR method was used to measure LMO2 ligand interaction (Bataille et al., 2020). WaterLOGSY experiments were conducted at a ¹H frequency of 600 MHz using a Bruker Avance spectrometer equipped with a BBI probe. All experiments were conducted at RT. 3 mm diameter NMR tubes with a sample volume of 200 μL were used for all experiments. For LMO2-LID, solutions were buffered using a 10 mM NaPO₄, 250 mM NaCl solution. For LMO2-ΔLID, solutions were buffered using a 10 mM NaPO₄, 50 mM NaCl solution. The sample preparation for measuring ligand binding with LMO2-LID is exemplified as follows; the compound (10 μL of a 10 mM solution in DMSO-d₆) was added to an Eppendorf tube before sequential addition of the appropriate buffer (90 μL), D₂O (20 μL), and the protein (80 μL, 25 μM). The resulting solution was vortexed to mix and transferred to a 3 mm NMR tube prior to the NMR analysis. The sample preparation for measuring ligand binding with LMO2-ΔLID is exemplified as follows; the compound (10 μL of a 10 mM solution in DMSO-d₆) was added to an Eppendorf tube before sequential addition of the appropriate buffer (162 μL), D₂O (20 μL), and the protein (8 μL, 250 μM). The resulting solution was vortexed to mix and transferred to a 3 mm NMR tube prior to the NMR analysis. The sample preparation for checking possible aggregation with the ligand alone is exemplified as follows; the compound (10 μL of a 10 mM solution in DMSO-de) was added to an Eppendorf tube before sequential addition of the appropriate buffer (170 μL) and D₂O (20 μL). The resulting solution was vortexed to mix and transferred to a 3 mm NMR tube prior to the NMR analysis.

### PAL Pulldown

20 μM of Abd-L26 with or without 100 μM of Abd-L9 (competitor) are added in a final volume of 400 μL of PBS with 40 μg of purified protein of interest (scFv-LMO2). The same samples are prepared for the no UV controls. The samples are incubated for 25 minutes at RT. The samples to be crosslinked are put into ice and under the UV lamp for crosslinking for 1 hour. The no UV controls are kept on ice. During the 1 hour of crosslinking, the agarose monomeric avidin beads (Cat #20228, Thermofisher) are washed twice with PBS. After the crosslink, 20 μL of washed beads are added in all the samples (crosslinked and non-crosslinked) and incubated for 2 hours at 4° C. on a roller. 2 hours later, wash the beads three times with 400 μL of PBS. The samples are finally denatured with 50 μL of 2× loading buffer with BME added directly on the beads (and boiled at 100° C. for 5 minutes) and loaded for a western blot analysis.

### CACO-2 and PAMPA Assays

Caco-2 apparent permeability (Papp) was determined in the Caco-2 human colon carcinoma cell line as described (Bavetsias et al., 2016). Cells were maintained (DMEM with 10% fetal bovine serum, penicillin, and streptomycin) in a humidified atmosphere with 5% CO₂/95% air for 10 days. Cells were plated out onto a cell culture assembly plate (Millipore, UK), and monolayer confluency was checked using a TEER electrode prior to the assay. Media was washed off and replaced with HBSS buffer (pH7.4) containing compound (10 μM, 1% DMSO) in the appropriate apical and basal donor wells. HBSS buffer alone was placed in acceptor wells. In particular instances a specific P-gp inhibitor, LY335979 (5 μM), was added to the HBSS. The Caco-2 plate was incubated for 2 hours at 37° C. Samples from the apical and basolateral chambers were analysed using a Waters (Milford, MA, US) TQ-S LC-MS/MS system. The cell permeability properties of Abd-L compounds was compared to low (nadolol) and high (antipyrine) permeability compounds and a compound with high export (indinavir).

Apparent permeability (Papp) was determined as follows:

$${P}_{app} = \frac{Vr ( mL )}{A ( {cm}^{2} ) \times C 0 ( μM )} \times Rate of Diffusion ( μM . {sec}^{- 1} )$$

$$\begin{matrix}Where Vr = volume of receptical \\ A = surface area of monolayer \\ C 0 = Initial compound concentration in donor\end{matrix}$$

### PAMPA

The parallel artificial membrane permeability assay (PAMPA) was used to determine compound permeability by passive diffusion. The assay used an artificial membrane consisting of 2% phosphatidyl choline in dodecane (Sigma Aldrich, Dorset, UK). The donor plate was a MultiScreen-IP Plate with 0.45 μm hydrophobe Immobilon-P Membrane (Millipore, UK) and the acceptor plate was a MultiScreen 96-well Transport Receiver Plate (Millipore, UK). The permeability was measured at 3 different pH levels: pH 5, 6.5 and pH 7.4 in buffer containing 1% Bovine Serum Albumin (Sigma Aldrich, Dorset, UK). A 10 mM DMSO stock solution of test compound was used to prepare the 10 μM PAMPA donor solutions and calibration curves in each of the three buffers.

6 μL of the membrane solution was added to each well of the donor plate. Buffer donor solutions (200 μL) were added to the appropriate wells of the PAMPA donor plate. 300 L per well of blank PBS (pH 7.4) was added to the PAMPA acceptor plate.

The donor and acceptor plates were then sandwiched together, covered with a lid and incubated at 30° C. in a humid environment for 16 hours. After the incubation period the plates were removed from the incubator and the sandwich was dismantled. Samples were then transferred into a fresh plate and centrifuged. All sample supernatants were diluted and analysed using a Waters (Milford, MA, US) TQ-S LC-MS/MS system.

Permeability values (cm/s) were calculated using the following equation:

$$\begin{matrix}{P}_{app} = C \times - ln ( 1 - \frac{[ {drug}_{acceptor} ]}{[ {drug}_{equilibrium} ]} ) \\ where C = \frac{{V}_{D} \times {V}_{A}}{( {V}_{D} + {V}_{A} ) \times area \times time}\end{matrix}$$

$$\begin{matrix}{V}_{D} = volume of donor \\ {V}_{A} = volume of acceptor \\ Area = surface area of the membrane \times porosity\end{matrix}$$

### Results

In Vitro cSPR Screening of a Chemical Library with an LMO2-iDAb Fusion Protein

The use of a high affinity intracellular antibody binding to RAS protein in a competitive SPR screening of a chemical library screen has previously been described (Quevedo et al., 2018) and relies on high affinity interaction between antibody and antigen on the SPR chip to select Abd compounds. The same approach was adopted with an intracellular antibody (in the form of an iDAb) binding to the LMO2 protein. Since, in this case, the interaction affinity is the nM range, rather than pM as the anti-RAS, a fusion between LMO2 and the iDAb where the two components were joined in a single polypeptide by a short flexible glycine-serine (GS) linker was used (FIG. 7A compared to the structure of LMO2 from PDB 2XJY in FIG. 7B). LMO2 can also be expressed in E. coli in complex with the LID domain of LIM domain binding 1 (LDB1) as this has been shown to be the only effective way to express soluble LMO2 protein (Ryan et al., 2006) apart for co-expressing with the iDAb. The binding of the iDAb to LMO2 occurs across the LMO2 hinge overlapping to some extent with LDB1 LID binding (Sewell et al., 2014). Consequently, a truncated version of the LID domain was co-expressed with LMO2 (hereafter LMO2-ΔLID) that covers the whole LIM2 of LMO2 but leaves the hinge and LIM1 regions accessible (FIG. 7C compared to the structure of LMO2-LID from PDB 2XJY in FIG. 7D). Therefore, an SPR chip was prepared with the LMO2-iDAb in one channel, LMO2-ΔLID in a second channel and a non-relevant protein, KRAS, was attached to a third channel. The chip format is shown in FIG. 1A. This organization of the SPR channels enabled sub-division of the chemical matter into those that might bind LMO2 in the iDAb binding region (Abd compounds as iDAb surrogates), those that bind LMO2 elsewhere than the LID and iDAb binding regions and those that bind to RAS.

Among the 1,500 screened compounds, four LMO2-ΔLID Abd hits were identified as having response units (RU) above 10 that did not bind the LMO2-iDAb or KRAS (FIG. 1B). The chemical structures and molecular weights of these are shown in FIG. 1C with the RU data from the screen. The compounds designated Abd-L1 and Abd-L4 were available commercially but Abd-L2 (an analogue of Abd-L1) and Abd-L3 were not. Further characterisation of Abd-L1 was undertaken using an orthogonal assay, 1D NMR waterLOGSY (Dalvit et al., 2001, Bataille et al., 2020) with LMO2-LID and LMO2-ΔLID. Proton peaks showed the polarity shifts caused by interaction with the LMO2-ΔLID whereas limited change was observed with the LMO2-LID protein (FIG. 1D).

The purpose of compound library screens is to identify chemical matter that can form the basis of drug development and therefore function in cells. Accordingly, the cell permeability properties of Abd-L1 in a CaCo-2 assay compared to low (nadolol) and high (antipyrine) permeability compounds and a compound with high export were assessed (indinavir) (FIG. 1E). The Caco-2 data show that Abd-L1 has low cellular permeability with high efflux in Caco-2 cells (FIG. 1E), which are poor cell-based drug properties.

### Establishing a BRET-Based LMO2-iDAb Biosensor for a Small Molecule Screen

Since the in vitro selection assays clearly do not necessary yield cell permeable compounds, an alternative approach was designed using a cell-based screening method for iDAb surrogates to improve cell properties of chemical hits (i.e. cell uptake, low export, etc). Such a cell-based screen for compounds that inhibit PPIs requires an assay that generates a signal from the PPI but which does not occur via a high affinity interaction because initial chemical hits would be expected to be weak binders. Accordingly, a BRET-based LMO2/iDAb LMO2 biosensor was engineered based on the strategy of RAS biosensors (Bery et al., 2018). Structural data for LMO2-iDAb LMO2 complex (Sewell et al., 2014) was used to optimise the proximity of donor and acceptor moieties. The donor moiety RLuc8 was fused at the carboxy terminal end of LMO2 and the GFP² acceptor molecule to the amino terminal end of the iDAb LMO2. The interaction between LMO2-RLuc8 and GFP²-iDAb LMO2, the lower affinity GFP²-iDAb LMO2_{dm} (a dematured iDAb LMO2 (Bery et al., 2018)) or the non-relevant GFP²-iDAb RAS (Tanaka et al., 2007) (hereafter named iDAb control or iDAb Ctl) was tested by BRET donor saturation assays (FIG. 8A). These data demonstrate that the dematuration mutagenesis has lowered the affinity of the iDAb LMO2_{dm} since there is 10-fold increase in BRET₅₀ (an approximation to the relative affinity of the acceptor for the donor protein) of iDAb LMO2_{dm} compared to iDAb LMO2 (0.44 versus 0.03 respectively, see FIG. 8A). The specificity of these interactions was assessed with a BRET competition assay in which an untagged competitor (iDAb LMO2) or a non-relevant competitor (iDAb Ctl) were expressed with either the BRET pairs LMO2-iDAb LMO2 (FIG. 8B) or LMO2-iDAb LMO2_{dm} (FIG. 8C). The competitor iDAb LMO2 decreased LMO2-iDAb LMO2 interaction in a dose dependant manner but only to ˜65% at the highest dose of competitor (FIG. 8B). Accordingly, iDAb LMO2 competed the lower affinity iDAb LMO2_{dm}-LMO2 interaction with a stronger inhibition at its highest dose (˜80%, FIG. 8C) and the expression of these proteins was not altered (FIG. 8D).

A dematuration method was employed to decrease iDAb affinity based on CDR sequences (Assi et al., 2010) such as it enabled an Alpha-Screen of RAS^{G12v}-binding compounds and analysis of in vitro derived RAS-binding Abd compounds (Tanaka & Rabbitts, in preparation). Based on the LMO2-iDAb LMO2 structural information (Sewell et al., 2014), we introduced additional mutations on the CDRs of iDAb LMO2_{dm} that would affect the interaction between key amino acids from the iDAb and LMO2 with alanine or glycine substitution while still retaining specific binding (FIG. 9A-C). Most of the modifications on the iDAb LMO2 affected its binding around the hinge region of LMO2 (FIG. 9A-C). Six additional mutants named iDAb LMO2_{dm1-6} (DNA and protein sequences shown in FIG. 10A-H) were constructred and tested in BRET donor saturation assays (FIG. 2A). Each of the iDAb LMO2 mutants (dm1-6) had a decreased BRETₘₐₓ value (an approximation for the total number of complex LMO2/iDAb and the distance between the donor and the acceptor within the dimer) and an increased BRET₅₀ value compared to the template iDAb LMO2_{dm} (FIG. 2B). This suggested an overall decreased affinity of the dematured iDAbs towards LMO2 and the mutations did not affect their expression (FIG. 2C). Finally, a BRET competition experiment was performed with each mutant with the lowest dose of competitor (FIG. 2D) to determine the optimal dematured iDAb for the chemical library screen. The competition data with iDAb LMO2_{dm3} showed it was the best mutant as its interaction with LMO2 was almost completely inhibited by iDAb LMO2 (˜90%) while it retained a relatively high BRET signal (FIG. 2D). Therefore, this mutant was selected for a cell-based high throughput screening of small molecules.

### High-Throughput Screen for Inhibitors of LMO2-iDAb_{dm3} Interaction

The robustness and scalability of the cell-based BRET LMO2-iDAb LMO2_{dm3} interaction assay was tested in a high-throughput screen (HTS) to identify compounds that inhibit this interaction. A library of 10,720 small molecules assembled from Biofocus and Chembridge sources were screened. The flowchart of the HTS is described in FIG. 3A. HEK293T cells were transfected on day 1 with plasmids expressing LMO2-RLuc8 and GFP²-iDAb LMO2_{dm3} and 24 hours later, compounds were added to 10 μM and the BRET signals determined after a further 24 hours. Thirty-four primary hits were identified using a cut-off of 3×SD from DMSO controls (Lavoie et al., 2013) (FIG. 3B). These were re-tested using the original BRET assay to confirm inhibition of signal and also using BRET-based interaction assay between LMO2 and unmutated iDAb LMO2 to eliminate compounds binding to the iDAb (FIG. 3C, D). In addition, initial hits affecting RLuc8 luminescence or intrinsic GFP² fluorescence by greater than two-fold were not considered further (FIG. 11A, B). This rescreen resulted in eight inhibitors of LMO2-iDAb LMO2_{dm3} interaction (FIG. 11C, D). The eight compounds were finally tested with a non-relevant BRET-based interaction assay (MAX bHLH-CMYC bHLH) to provide further confirmation of specific interaction with LMO2 (FIG. 11E). The chemical structures of the selected hits show that the compounds belong to a family that can be divided into two subfamilies due to their chemical similarities. The main difference is the presence of either a 5 or 7 membered ring in each sub-family (FIG. 11C, D, respectively).

### Structure-Activity Relationship Study of Abd Compounds

Samples of the 5-membered ring-containing hits (FIG. 11C) were prepared following literature methods. However, it was found that the core underwent a rearrangement to the 5-membered when attempting to synthesise samples of 7-membered ring-containing compounds (FIG. 11D). Therefore, the corresponding 5-membered ring analogues, which were named Abd-L5 to Abd-L12 were synthesised (FIG. 4A). These analogues were tested in a dose-response BRET assay to verify their ability to bind LMO2 and their potency (FIG. 4B and FIG. 12A). Three of the analogues, Abd-L5, Abd-L8 and Abd-L11, were unable to inhibit LMO2-iDAb LMO2_{dm3} interaction (FIG. 4B). The most potent inhibitor, Abd-L9, was used as a template for a structure-activity relationship study (FIG. 4C, D).

The different moieties of Abd compounds were divided into 4 substituent groups, namely benzyl (position A), imidazolidinone (B), oxazole (C), and aniline (D) (FIG. 4C) and were modified systematically. Representative analogues are shown (Abd-L13 to Abd-L25, FIG. 4D and FIG. 12B-F). Most substituted benzyl groups were found to be well tolerated in position A, the methoxy group could be placed in alpha, meta or para positions on the benzyl ring with minimal effect on the BRET inhibition potency of derivatives (compounds with benzyl modifications (red boxes) and/or aniline (green boxes) modifications are shown in FIG. 4C). It was found that a large array of substituted anilines and benzyl amines were also well tolerated at position D. It is noteworthy that most substituted pyridine-containing compounds did not exhibit activity, which result from the change of basicity in those compounds.

Modifications to positions B and C had more substantial effect on the potency of the analogues. In position B, any replacement of the imidazolidinone was found to bring a loss of activity (as such pyrimidinone and piperazinones, FIG. 12E, F) apart from the corresponding piperazine (pink box in FIG. 4D). Due to the potential chemical instability of the imidazolidinone, the lower yields and a large number of side-products during synthesis, a piperazine moiety was substituted for further SAR investigations. Those issues were eradicated with the change to the piperazine. In position C, it was found that only the 2,4-substituted-thiazole and 2,4-substituted-oxazole were tolerated as a core and the corresponding 2,5-substituted heterocycles and different heterocycle (such as pyrimidine) led to a loss of activity (see blue box on the position C of the analogues in FIG. 4D and FIG. 12E, F). These data suggest that the B/C positions are important for the interaction of the compounds with LMO2 while position A/D could be modified to add new functional groups.

Abd-L9 and some analogues were tested in a Parallel Artificial Membrane Permeability Assay (PAMPA) and Abd-L9 in a Caco-2 permeability assays (FIG. 13A, B). This showed the compounds were permeable through a synthetic membrane (PAMPA) or into cells (Caco-2) as would be expected compounds derived from cell-based screens. Abd-L9 showed the best properties in the PAMPA compared to the analogues and a low transport but low efflux ratio in the Caco-2 assay (FIG. 13A, B). These results suggest that while Abd-L9 enters cells with a relatively low efficiency, it is not actively exported from the cells (low efflux ratio).

### Abd Compounds Bind LMO2 In Vitro

The Abd compounds were identified and verified in cell-based assays. LMO2-LID and LMO2-ΔLID were used in waterLOGSY NMR experiments to assess the binding of small molecules with LMO2. One compound from the cSPR screen (Abd-L1, FIG. 1D) and three LMO2 compounds from the cell-based screen were tested (Abd-L9, Abd-L10 and Abd-L13, FIG. 5A-C) and each showed binding to LMO2-ΔLID but not LMO2-LID. Since the difference between the two proteins is the hinge and LIM1, the data concur with cell data that the compounds bind LMO2 and that they bind on the interface restricted to the LIM1 and the hinge region of LMO2.

These data were further confirmed by using an alternative method: the photoaffinity labelling (PAL), a powerful technique used to study protein-ligand interactions (Smith and Collins, 2015). PAL is the use of a chemical probe that can covalently bind to its target in response to activation by light (Sadakane and Hatanaka, 2006). The extensive SAR data on the LMO2 Abd compounds, suggested attachment sites on the parent ligand. A benzophenone photoreactive group was added in place of the benzyl substituent (position A) and a linker with a biotin tag in position D (FIG. 5D). In order to obtain soluble, recombinant LMO2 protein that has an Abd-L compound-binding site accessible, A phage display screen of scFvs was carried out with LMO2-LID protein antigen and obtained scFv that binds LMO2 and can be co-expressed in E. coli(Miller & Rabbitts, unpublished). By employing the partially purified LMO2-scFv dimer, the PAL technique was performed after inducing crosslinking of a photoaffinity LMO2 PAL compound (designated Abd-L26, that comprises a photo-active substituent and a biotin moiety attached to the compound with a short linker; FIG. 5D) to the scFv-LMO2 complex with UV light for photo-crosslinking. The Abd-L26 in the complex was isolated by interaction of the biotin moiety with avidin beads and the protein analysed by Western blot with either anti-biotin antibody (FIG. 5E), anti-LMO2 antibody (FIG. 5F) or anti-HIS tag (FIG. 5G). The pulldown data show that protein is only crosslinked when the mixture is treated with UV light and we observed a biotin labelled protein (FIG. 5E, lane 3) coincident the size of LMO2 (FIG. 5F). In addition, the recovery of biotinylated LMO2 was inhibited by incubating the protein with Abd-L26 (PAL) in the presence of 5× concentration of Abd-L9 competitor (FIG. 5E, lane 4). The anti-biotin antibody showed that the biotinylated proteins specifically bound to the beads through the PAL Abd-L26 compound while the anti-LMO2 and anti-HIS antibodies show non-specific binding of proteins to the beads. It was noted that the recombinant LMO2 had a tendency to associate non-specifically as well as the scFv with avidin agarose beads used for the pulldown (see lanes 1 & 2, FIG. 5F, G). This is possibly due to partial denaturation of the proteins during the PAL incubation and may explain the apparent partial inability of Abd-L9 to compete the PAL compound (FIG. 5F, lane 4 versus lane 3).

### Activity of LMO2 Abd Compounds in Cells

The specificity and potency of Abd-L compounds in cells by using dose-response BRET assays on different LMO2 PPI was tested. This included LMO2 interaction with the unmutated iDAb and the iDAb_{dm3}, with its natural partner proteins LDB1 and TAL1 (together with E47) (Wadman et al., 1997) and with a non-relevant control PPI which is the interaction of the bHLH regions of CMYC with MAX. In order to develop the various BRET assays, the direct interaction LMO2 with TAL1 by a BRET donor saturation assay was initially tested (FIG. 14A) but this interaction is weak and gave a high BRET₅₀ value. Partner proteins involved in the LMO2 complex (Wadman et al., 1997) were added individually and it was found that co-expression of E47, a heterodimerization partner of TAL1, increased the relative affinity of LMO2-TAL1 and the addition of LDB1 gave the strongest binding between LMO2 and TAL1 (FIG. 14A, see decreasing BRET₅₀ values: from 12.6 to 1). The BRET pairs LMO2-LDB1 (FIG. 14B) were also developed as well as the non-relevant interaction of MAX bHLH with CMYC bHLH (FIG. 14C). Finally, the specificity of these three interactions was tested, with BRET competition assays, by co-expressing non-tagged versions of iDAb Ctl or iDAb LMO2 in the BRET assay cells. iDAb LMO2 inhibited BRET signal from LMO2-TAL1+E47 (FIG. 14D) and from LMO2-LDB1 (FIG. 14E) but not from MAX-CMYC interaction (FIG. 14F).

The anti-LMO2 Abd compounds were assessed in BRET dose-response assays with the various BRET assays (FIG. 6A-D and FIG. 14G, H). None of the compounds inhibited LMO2-iDAb LMO2_{dm3} BRET by more than 40-50%, with the exception of Abd-L22 (˜85%). However, it was found that Abd-L9, Abd-L10 and Abd-L16 had the best IC₅₀ for the interaction LMO2-iDAb LMO2_{dm3} at around 1 μM (FIG. 6A and Table 1) whether or not the compound contained imidazolidinone substituents (Abd-L9 and L10) or a piperazine substituent (Abd-L16). The other compounds tested showed IC₅₀ values ranging from just over 7 μM to nearly 50 μM for Abd-L19 (FIG. 6A, D and Table 1). When this group of compounds were assayed with LMO2-LDB1 BRET, little effect was observed except Abd-L10 which caused only a small inhibition (35% at the highest concentration of Abd-L10 with an IC₅₀ of 1.2 μM, Table 1 and FIG. 6B, D). Testing this group of Abd-L compounds with the BRET assays for LMO2-iDAb LMO2 (unmutated iDAb) (FIG. 6C), LMO2-TAL1+E47 (FIG. 14G) or MAX bHLH-CMYC bHLH (FIG. 14H) failed to show any inhibition, even at the highest concentration of compound as used throughout the series of BRET inhibition assays with the exception of Abd-L22 that inhibits LMO2-iDAb LMO2 interaction only at high concentrations (above 25 μM, FIG. 6C).

### Intracellular Antibodies as Templates for Drug Discovery

Intracellular antibody fragments interact with proteins at any antigenic site or where natural partner proteins are involved in PPI. This gives an opportunity using the intracellular antibody to derive compounds that are surrogates for the specific interaction residues with the intracellular antibody. When the intracellular antibody interferes directly with a PPI, rather than employing the natural partner protein, the intracellular antibody can be obtained with very high affinity binding, as shown for selected compounds binding to the RAS proteins (Quevedo et al., 2018) demonstrating that this so-called undruggable target is in fact druggable. The in vitro method, using intracellular antibodies as tools for drug discovery, employs competitive SPR with a chip carrying a target protein with interacting antibody in place (Quevedo et al., 2018). The RAS-binding compounds were successful due to the very high affinity of the anti-RAS antibody limiting loss of antibody-antigen interaction on the SPR chip. As described herein, the similar approach using an anti-LMO2 iDAb was implemented and circumvented the problem of loss of iDAb during the library screen by linking LMO2 and iDAb with a flexible linker. In this way, LMO2-binding chemical matter was identified. Examination of the cell-based properties of one of these compounds showed disadvantageous features.

Alternatively, in cell-based assays such as the BRET assay described herein, the affinity of the iDAbs for their target is not a limitation. Furthermore, the ability to carry out affinity manipulation on tight binders is facile with antibodies because only the primary sequence identifies the CDRs for mutagenesis in a process called intracellular antibody dematuration (Tanaka & Rabbitts, in preparation). This process does not require structural information and iDAbs of interest with lower binding properties could be directly used in this cell-based approach, which makes this a flexible approach. In addition, cell-based screening is also a more versatile approach as it can be implemented to any protein that is difficult to express, such as LMO2 which has eluded recombinant expression except in co-expression with LDB1 LID (Ryan et al., 2006) or the iDAb (Sewell et al., 2014). Finally, the intrinsic advantage of cell-based assays, in which a signal is generated by the direct interaction of target with iDAb, is that the compounds already have the characteristic of cell entry, which we show here with our LMO2 Abd-L series of compounds.

LMO2 Binding Compounds Derived from a Cell-Based BRET2 Chemical Library Screen

A cell-based intracellular single domain antibody-guided small molecule selection method as described herein allows the direct identification of compounds that bind at the same region of the iDAb. This has been illustrated using the T cell oncogenic protein LMO2. LMO2 encodes a 18 kDa polypeptide that comprises two zinc-binding LIM domains (Chambers and Rabbitts, 2015). These domains are the interface for binding to class II basic helix-loop-helix (bHLH) transcription factors such as TAL1/E2A and GATA (Wadman et al., 1994). Furthermore, these two DNA-binding complexes are bridged by a scaffolding protein, LDB1 that binds LMO2 on a different interface than the transcription factors (Wadman et al., 1997). An anti-LMO2 iDAb has been characterised that inhibits the tumourigenic function of LMO2 in vivo as it prevents LMO2-dependent tumour growth in a transplantation assay mediated by the disruption of the LMO2-multimeric complex by preventing LDB1 interaction (Tanaka et al., 2011). In detail, the anti-LMO2 intracellular antibody functions as an indirect PPI inhibitor by a new mechanism, which is altering the natural structure of LMO2. The iDAb LMO2 induces a change of conformation between the two LIM domains of LMO2 that is not compatible with the interaction of LDB1 and the transcription factors (Sewell et al., 2014). With the Abd-L compounds selected here, no significant modification of the conformation of LMO2 protein was observed as shown with BRET data employing the TAL1/E47: while the iDAb LMO2 impedes the binding of these proteins with LMO2 (FIG. 14D), the Abd-L compounds did not (FIG. 14G).

The iDAb was employed to screen a compound library (10K compounds) that bind to LMO2 in the LMO2-iDAb BRET2 cell-based interaction assay. A number of initial hits were obtained and one chemical series of which Abd-L5 to Abd-L12 were the progenitors. Direct binding of compounds Abd-L9, Abd-L10 and Abd-L13 using recombinant LMO2-ΔLID proteins in waterLOGSY NMR was confirmed (FIG. 5A-C). SAR analysis was carried out on the chemical series to determine if the compounds could tolerate larger groups and linkers, and therefore allowing the use of PAL technology. The use of a benzophenone moiety was tested as a photoaffinity label and observed that analogues bearing this group on the right- or the left-hand sides were still active. The linker however could not be located on the left-hand side as this caused loss of activity. A compound was therefore prepared with the benzophenone photoreactive moiety on the piperazine and the biotin linked to the aniline in the para position via a small polyethylene glycol (PEG) chain, generating compound Abd-L26 (FIG. 5D). The cross-linking of Abd-L26 to the LMO2 protein confirmed binding to LMO2 protein (FIG. 5E-F) and this was inhibited by addition of Abd-L9. These data support the conclusion that the chemical series is an intracellular antibody surrogate that bind to LMO2 where the anti-LMO2 iDAb contacts LMO2. The cell-based selection involved competition by the compounds for the interaction of LMO2 with a dematured iDAb and these compounds do not influence the interaction of LMO2 with unmutated iDAb (except Abd-L22 at the highest concentrations) as would be expected from their M interference IC₅₀. In addition, the compounds do not bind to LMO2-LID fusion whereas they bind to an LMO2-ΔLID fusion (where the LID is truncated for part of the region where the iDAb binds) further defining their binding site on LMO2 as the iDAb interaction region.

### Synthesis

Several methods for the chemical synthesis of heterocyclic carboxamide compounds of the present application are described herein. These and/or other well-known methods may be modified and/or adapted in various ways in order to facilitate the synthesis of additional compounds within the scope of the present application and claims. Such alternative methods and modifications should be understood as being within the spirit and scope of this application and claims. Accordingly, it should be understood that the methods set forth in the following descriptions, schemes and examples are intended for illustrative purposes and are not to be construed as limiting the scope of the disclosure.

All solvents and reagents were used as supplied (analytical or HPLC grade) without prior purification. Water was purified by an Elix® UV-10 system. Thin layer chromatography was performed on aluminium plates coated with 60 F254 silica. Plates were visualised using UV light (254 nm) or 1% aq. KMnO4. Flash column chromatography was performed on Kieselgel 60M silica in a glass column. NMR spectra were recorded on Bruker Avance spectrometers (AVII400, AVIII 400, AVIIIHD 600 or AVIII 700) in the deuterated solvent stated. The field was locked by external referencing to the relevant deuteron resonance. Chemical shifts (δ) are reported in parts per million (ppm) referenced to the solvent peak. ¹H spectra reported to two decimal places, and ¹³C spectra reported to one decimal place, and coupling constants (J) are quoted in Hz (reported to one decimal place). The multiplicity of each signal is indicated by: s (singlet); br. s (broad singlet); d (doublet); t (triplet); q (quartet); dd (doublet of doublets); td (triplet of doublets); qt (quartet of triplets); or m (multiplet). Low-resolution mass spectra (LRMS) were recorded on an Agilent 6120 spectrometer from solutions of MeOH. Accurate mass measurements were run on either a Bruker MicroTOF internally calibrated with polyalanine, or a Micromass GCT instrument fitted with a Scientific Glass Instruments BPX5 column (15 m×0.25 mm) using amyl acetate as a lock mass, by the mass spectrometry department of the Chemistry Research Laboratory, University of Oxford, UK.; m/z values are reported in Daltons.

### General Procedure A: Synthesis of Substituted Imidazolidinones (n=1) and Pyrimidinones (n=2)

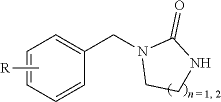

The requisite cyclic urea (1.0 eq.) was dissolved in THF (10 mL) and cooled to 0° C. before portionwise addition of NaH (60% suspension in oil, 1.0 eq.). After 30 min, the suspension was treated with the requisite substituted benzylbromide/chloride (0.9 eq.). The resulting mixture was stirred for 2 h (monitoring by LC-MS and TLC) and warmed to room temperature, before addition of NH₄Cl (sat. aq. sol., 20 mL) and EtOAc (20 mL). The aqueous layer was extracted with EtOAc (2×20 mL), the combined organic phase was washed with water (20 mL), brine (20 mL of saturated aqueous solution of sodium chloride), dried (Na₂SO₄), filtered and concentrated in vacuo (use of a rotary evaporator attached to a diaphragm pump). The crude material was purified on silica gel (5% MeOH in CH₂Cl₂) and the desired compound was obtained as a colourless oil that solidified on standing.

### General Procedure B: Synthesis of Substituted Piperazines

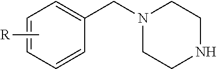

Boc-piperazine (1.1 eq.) was dissolved in MeCN (5 mL) before sequential addition of K₂CO₃ (2.5 eq.) followed by the requisite substituted benzylbromide/chloride (1.0 eq.). The resulting mixture was stirred for 18 h before addition of H₂O/brine (1:1, 20 mL) and EtOAc (20 mL). The aqueous layer was extracted with EtOAc (20 mL), the combined organic phase was dried (Na₂SO₄), filtered and concentrated in vacuo. The crude material was purified on silica gel (10% EtOAc in pentane) and the title compound was obtained as a colourless oil that solidified on standing. The product was dissolved in CH₂Cl₂ (5 mL) before addition of TFA (500 μL). The resulting solution was stirred for 18 h at room temperature and concentrated in vacuo. The compound was used in the next step without further purification.

### General Procedure C: Palladium Coupling of Substituted Cyclic Ureas to Chlorooxazoles (X═O) and Chlorothiazoles (X═S)

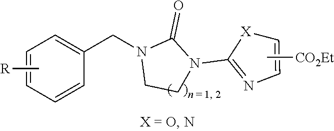

The requisite substituted cyclic urea (1.1 eq.), Cs₂CO₃ (3.0 eq.), the ester substituted chloro heterocycle of choice (1.0 eq.) and X-Phos (10% mol) were added sequentially to a microwave vial followed by degassed 1,4-dioxane (2 mL). The suspension was degassed for 5 min with nitrogen before addition of Pd(OAc)₂ (5% mol); it was degassed further with nitrogen for another 5 min before the vessel was sealed and the suspension heated to 95° C. for 24 h. The reaction was cooled down, diluted with EtOAc (10 mL) and washed with brine/water (1:1, 10 mL). The organic phase was dried (Na₂SO₄), filtered and concentrated in vacuo. The crude material was purified on silica gel to give the desired compound.

### General Procedure D: Synthesis of Substituted Pyperazyl Hererocycles

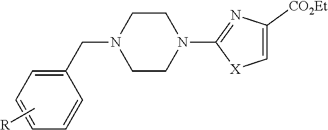

The substituted piperazine (1.2 eq.) was dissolved in 1,4-dioxane/N,N-diisopropylethylamine (4:1, 8 mL) before addition of the requisite chloro-heterocycle (1.0 eq.). The solution was stirred at 60° C. for 48 h, cooled to room temperature, diluted with EtOAc (30 mL) and washed with H₂O/brine (1:1, 20 mL). The organic phase was dried (Na₂SO₄), filtered and concentrated in vacuo. The crude material then purified on silica gel to give the title compound.

### General Procedure E: Basic Hydrolysis of the Ester Moiety and Subsequent HATU Amide Coupling

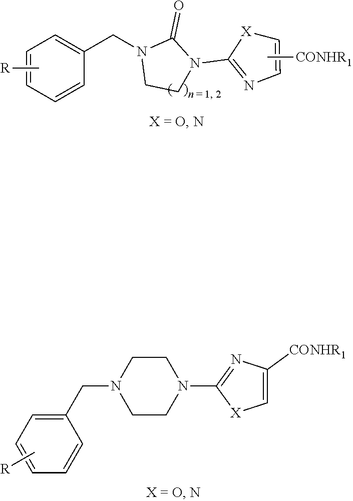

The ester (1.0 eq.) was dissolved in THF/MeOH (4:1) before addition of NaOH (1M aq.) until pH>8. The resulting reaction was stirred for 16 h at room temperature, after which it was acidified with HCl (1M aq.) until pH<5. The solution was concentrated in vacuo and the obtained carboxylic acid was used in the next step without further purification. The acid was dissolved in DMF (2 mL) before sequential addition of N,N-diisopropylethylamine (3.0 eq.), the requisite amine (1.2 eq.) and HATU (1.4 eq.). The resulting solution was stirred for 18 h, diluted with EtOAc (10 mL) and washed with brine/water (1:1, 3×50 mL). The organic phase was dried (Na₂SO₄), filtered and concentrated in vacuo. The crude material was purified on silica gel to give the title compound.

### EXPERIMENTAL DATA

### Abd-L5:1-benzylimidazolidin-2-one (1)

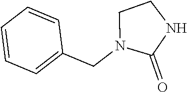 [SMILES: O=C1NCCN1Cc1ccccc1]

Following General Procedure A using 2-imidazolidinone (1.00 g, 11.6 mmol, 1.0 eq.) and benzyl bromide (1.25 mL, 10.4 mmol, 0.9 eq.), the title compound 1 was obtained as a colourless oil that solidified on standing (858 mg, 42%), after purification on silica gel (5% MeOH in CH₂Cl₂).

m/z LRMS (ESI⁺): 177 (100%) [M+H]⁺.

### ethyl 2-(3-benzyl-2-oxoimidazolidin-1-yl)thiazole-5-carboxylate (2)

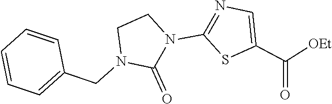 [SMILES: CCOC(=O)c1cnc(N2CCN(Cc3ccccc3)C2=O)s1]

Following General Procedure C using cyclic urea 1 (102 mg, 0.578 mmol, 1.1 eq.) and ethyl 2-chlorothiazole-5-carboxylate (100 mg, 0.525 mmol, 1.0 eq.), the title compound 2 was obtained as a yellow oil (125 mg, 72%), after purification on silica gel (3% MeOH in CH₂Cl₂). m/z LRMS (ESI⁺): 332 (100%) [M+H]⁺.

### 2-(3-benzyl-2-oxoimidazolidin-1-yl)-N-(2-(methylsulfonamido)phenyl)thiazole-5-carboxamide (3) (Abd-L5)

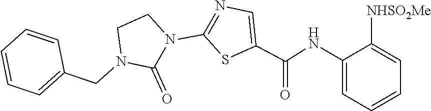

Following General Procedure E using ester 2 and N-(2-aminophenyl)methanesulfonamide, the title product was obtained as a thick yellow oil that solidified on standing (32 mg, 41%), after two purifications on silica gel (5% MeOH in CH₂Cl₂). m/z LRMS (ESI⁺): 472 (100%) [M+H]⁺. HRMS (ESI⁺): calc. for C₂₁H₂₂N₅O₄³²S₂[M+H]⁺ 472.1113. found 472.1120.

### Abd-L6: ethyl 2-(3-benzyl-2-oxoimidazolidin-1-yl)oxazole-4-carboxylate (4)

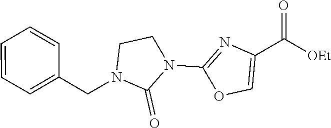 [SMILES: CCOC(=O)c1coc(N2CCN(Cc3ccccc3)C2=O)n1]

Following General Procedure C using cyclic urea 1 (110 mg, 0.629 mmol, 1.1 eq.) and ethyl 2-chlorooxazole-4-carboxylate (100 mg, 0.571 mmol, 1.0 eq.), the title compound 4 was obtained as a yellow oil (106 mg, 59%), after purification on silica gel (3% MeOH in CH₂Cl₂). m/z LRMS (ESI⁺): 316 (100%) [M+H]⁺. 2-(3-benzyl-2-oxoimidazolidin-1-yl)-N-(4-phenoxyphenyl)oxazole-4-carboxamide (5) (Abd-L6)

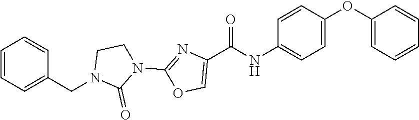 [SMILES: O=C(Nc1ccc(Oc2ccccc2)cc1)c1coc(N2CCN(Cc3ccccc3)C2=O)n1]

Following General Procedure E using ester 4 and 4-phenoxyaniline, the title product was obtained as a thick yellow oil that solidified on standing (27 mg, 52%), after purification on silica gel (5% MeOH in CH₂Cl₂). m/z LRMS (ESI⁺): 455 (100%) [M+H]⁺. HRMS (ESI⁺): calc. for C₂₆H₂₃N₄O₄ [M+H]⁺ 455.1719. found 455.1720.

### Abd-L7:1-(3-chlorobenzyl)imidazolidin-2-one (6)

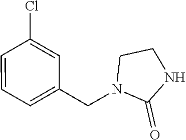 [SMILES: O=C1NCCN1Cc1cccc(Cl)c1]

Following General Procedure A using 2-imidazolidinone (500 mg, 5.80 mmol, 1.0 eq.) and 3-chlorobenzyl bromide (685 μL, 5.20 mmol, 0.9 eq.), the title compound 6 was obtained as a colourless oil that solidified on standing (463 mg, 38%), after purification on silica gel (3% MecOH in CH₂Cl₂). m/z LRMS (ESI⁺): 211 (100%) [M+H]⁺.

### ethyl 2-(3-(3-chlorobenzyl)-2-oxoimidazolidin-1-yl)oxazole-4-carboxylate (7)

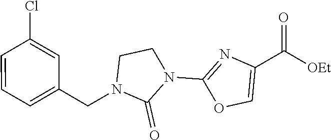 [SMILES: CCOC(=O)c1coc(N2CCN(Cc3cccc(Cl)c3)C2=O)n1]

Following General Procedure C using cyclic urea 6 (132 mg, 0.629 mmol, 1.1 eq.) and ethyl 2-chlorooxazole-4-carboxylate (100 mg, 0.571 mmol, 1.0 eq.), the title compound 7 was obtained as a yellow oil (117 mg, 54%), after purification on silica gel (3% MeOH in CH₂Cl₂). m/z LRMS (ESI⁺): 350 (100%) [M+H]⁺.

### N-(4-(1H-pyrrol-1-yl)phenyl)-2-(3-(3-chlorobenzyl)-2-oxoimidazolidin-1-yl)oxazole-4-carboxamide (8) (Abd-L7)

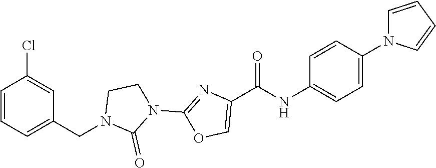 [SMILES: O=C(Nc1ccc(-n2cccc2)cc1)c1coc(N2CCN(Cc3cccc(Cl)c3)C2=O)n1]

Following General Procedure E using ester 7 and 4-pyrroleaniline, the title product was obtained as a thick yellow oil that solidified on standing (27 mg, 52%), after purification on silica gel (7% MeOH in CH₂Cl₂).

m/z LRMS (ESI⁺): 462 (100%) [M+H]⁺. HRMS (ESI⁺): calc. for C₂₄H₂₁³⁵ClN₅O₃[M+H]⁺ 462.1333. found 462.1332.

### Abd-L8:1-(4-chlorobenzyl)imidazolidin-2-one (9)

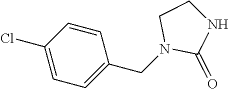 [SMILES: O=C1NCCN1Cc1ccc(Cl)cc1]

Following General Procedure A using 2-imidazolidinone (500 mg, 5.80 mmol, 1.0 eq.) and 4-chlorobenzyl bromide (1.07 g, 5.20 mmol, 0.9 eq.), the title compound was obtained as a colourless oil that solidified on standing (463 mg, 38%), after purification on silica gel (5% MeOH in CH₂Cl₂). m/z LRMS (ESI⁺): 211 (100%) [M+H]⁺.

### ethyl 2-(3-(4-chlorobenzyl)-2-oxoimidazolidin-1-yl)oxazole-5-carboxylate (10)

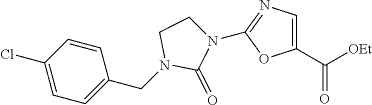 [SMILES: CCOC(=O)c1cnc(N2CCN(Cc3ccc(Cl)cc3)C2=O)o1]

Following General Procedure C using cyclic urea 9 (132 mg, 0.629 mmol, 1.1 eq.) and ethyl 2-chlorooxazole-5-carboxylate (100 mg, 0.571 mmol, 1.0 eq.), the title compound 10 was obtained as a yellow oil (122 mg, 61%), after purification on silica gel (3% MeOH in CH₂Cl₂).

m/z LRMS (ESI⁺): 350 (100%) [M+H]⁺.

### 2-(3-(4-chlorobenzyl)-2-oxoimidazolidin-1-yl)-N-(3,4-dimethoxyphenyl)oxazole-5-carboxamide (11) (Abd-L8)

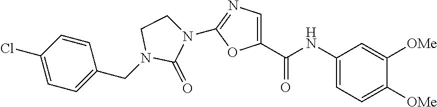 [SMILES: COc1ccc(NC(=O)c2cnc(N3CCN(Cc4ccc(Cl)cc4)C3=O)o2)cc1OC]

Following General Procedure E using ester 10 and 3,4-dimethoxyaniline, the title product was obtained as a thick brown oil that solidified on standing (22 mg, 54%), after purification on silica gel (5% MeOH in CH₂Cl₂). m/z LRMS (ESI⁺): 457 (100%) [M+H]⁺. HRMS (ESI⁺): calc. for C₂₂H₂₂₃5ClN₄O₅[M+H]⁺ 457.1279. found 457.1282.

### Abd-L9:1-(3-methoxybenzyl)imidazolidin-2-one (12)

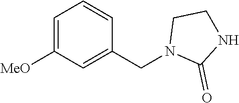 [SMILES: COc1cccc(CN2CCNC2=O)c1]

Following General Procedure A using 2-imidazolidinone (1.00 g, 11.6 mmol, 1.0 eq.) and 3-methoxybenzylbromide (1.47 mL, 10.5 mmol, 0.9 eq.), the title compound was obtained as a colourless oil that solidified on standing (1.02 g, 47%), after purification on silica gel (5% MeOH in CH₂Cl₂).

m/z LRMS (ESI⁺): 207 (100%) [M+H]⁺.

### ethyl 2-(3-(3-methoxybenzyl)-2-oxoimidazolidin-1-yl)oxazole-4-carboxylate (13)

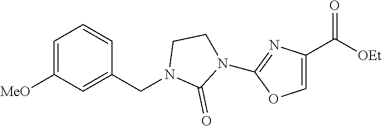 [SMILES: CCOC(=O)c1coc(N2CCN(Cc3cccc(OC)c3)C2=O)n1]

Following General Procedure C using cyclic urea 12 (82 mg, 0.396 mmol, 1.1 eq.) and ethyl 2-chlorooxazole-4-carboxylate (64 mg, 0.360 mmol, 1.0 eq.), the title compound 13 was obtained as a yellow oil (88 mg, 71%), after purification on silica gel (3% MeOH in CH₂Cl₂).

m/z LRMS (ESI⁺): 346 (100%) [M+H]⁺.

### N-(4-(benzyloxy)phenyl)-2-(3-(3-methoxybenzyl)-2-oxoimidazolidin-1-yl)oxazole-4-carboxamide (14) (Abd-L9)

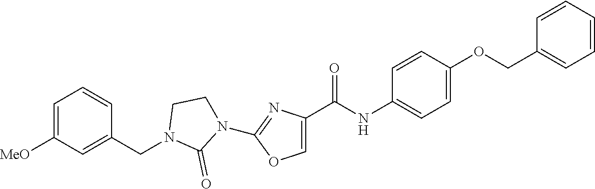 [SMILES: COc1cccc(CN2CCN(c3nc(C(=O)Nc4ccc(OCc5ccccc5)cc4)co3)C2=O)c1]

Following General Procedure E using ester 13 and 4-(benzyloxy)aniline, the title product was obtained as a dark yellow oil that solidified on standing (137 mg, 62%), after purification on silica gel (5% MeOH in CH₂Cl₂).

m/z LRMS (ESI⁺): 499 (100%) [M+H]⁺. HRMS (ESI⁺): calc. for C₂₈H₂₇N₄O₅ [M+H]⁺ 499.1981. found 499.1978.

### Abd-L10: 3-((2-oxoimidazolidin-1-yl)methyl)benzonitrile (15)

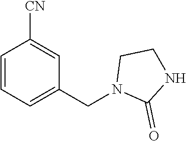 [SMILES: N#Cc1cccc(CN2CCNC2=O)c1]

Following General Procedure A using 2-imidazolidinone (500 mg, 5.80 mmol, 1.0 eq.) and 3-cyanobenzyl bromide (1.01 g, 5.20 mmol, 0.9 eq.), the title compound was obtained as a colourless oil that solidified on standing (463 mg, 41%), after purification on silica gel (5% MeOH in CH₂Cl₂).

m/z LRMS (ESI⁺): 202 (100%) [M+H].

### ethyl 2-(3-(3-cyanobenzyl)-2-oxoimidazolidin-1-yl)oxazole-4-carboxylate (16)

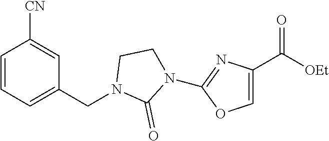 [SMILES: CCOC(=O)c1coc(N2CCN(Cc3cccc(C#N)c3)C2=O)n1]

Following General Procedure C using cyclic urea 15 (126 mg, 0.629 mmol, 1.1 eq.) and ethyl 2-chlorooxazole-4-carboxylate (100 mg, 0.571 mmol, 1.0 eq.), the title compound 16 was obtained as a yellow oil (109 mg, 56%), after purification on silica gel (3% MeOH in CH₂Cl₂). m/z LRMS (ESI⁺): 341 (100%) [M+H]⁺.

### N-(4-(1H-pyrrol-1-yl)phenyl)-2-(3-(3-cyanobenzyl)-2-oxoimidazolidin-1-yl)oxazole-4-carboxamide (17) (Abd-10)

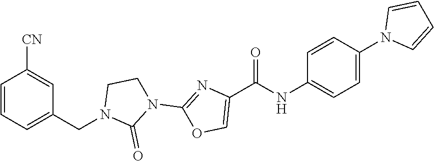 [SMILES: N#Cc1cccc(CN2CCN(c3nc(C(=O)Nc4ccc(-n5cccc5)cc4)co3)C2=O)c1]

Following General Procedure E using ester 16 and 4-pyrroleaniline, the title product was obtained as a thick yellow oil that solidified on standing (27 mg, 52%), after purification on silica gel (7% MeOH in CH₂Cl₂).

m/z LRMS (ESI⁺): 453 (100%) [M+H]⁺. HRMS (ESI⁺): calc. for C₂₅H₂₁N₆O₃ [M+H]⁺ 453.1675. found 453.1674.

### Abd-L11: 2-(3-(4-chlorobenzyl)-2-oxoimidazolidin-1-yl)-N-phenethyloxazole-5-carboxamide (18) (Abd-L11)

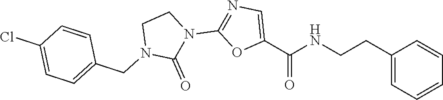 [SMILES: O=C(NCCc1ccccc1)c1cnc(N2CCN(Cc3ccc(Cl)cc3)C2=O)o1]

Following General Procedure E using ester 10 and 2-phenylethan-1-amine, the title product was obtained as a thick yellow oil that solidified on standing (18 mg, 49%), after purification on silica gel (5% MeOH in CH₂Cl₂).

m/z LRMS (ESI⁺): 425 (100%) [M+H]⁺. HRMS (ESI⁺): calc. for C₂₂H₂₂³⁵ClN₄O₃[M+H]⁺ 425.1380. found 425.1382.

### Abd-L12: 1-(2-chlorobenzyl)imidazolidin-2-one (19)

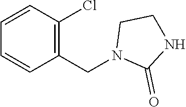 [SMILES: O=C1NCCN1Cc1ccccc1Cl]

Following General Procedure A using 2-imidazolidinone (500 mg, 5.80 mmol, 1.0 eq.) and 2-chlorobenzyl bromide (680 μL, 5.20 mmol, 0.9 eq.), the title compound was obtained as a colourless oil that solidified on standing (463 mg, 38%), after purification on silica gel (5% MeOH in CH₂Cl₂).

m/z LRMS (ESI⁺): 211 (100%) [M+H]⁺.

### ethyl 2-(3-(2-chlorobenzyl)-2-oxoimidazolidin-1-yl)oxazole-4-carboxylate (20)

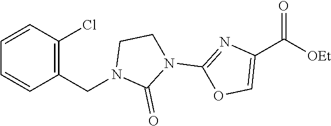 [SMILES: CCOC(=O)c1coc(N2CCN(Cc3ccccc3Cl)C2=O)n1]

Following General Procedure C using cyclic urea 19 (132 mg, 0.629 mmol, 1.1 eq.) and ethyl 2-chlorooxazole-4-carboxylate (100 mg, 0.571 mmol, 1.0 eq.), the title compound 20 was obtained as a yellow oil (98 mg, 49%), after purification on silica gel (3% MeOH in CH₂Cl₂).

m/z LRMS (ESI⁺): 350 (100%) [M+H]⁺.

### 2-(3-(2-chlorobenzyl)-2-oxoimidazolidin-1-yl)-N-(4-phenoxyphenyl)oxazole-4-carboxamide (21) (Abd-L12)

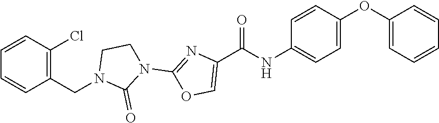 [SMILES: O=C(Nc1ccc(Oc2ccccc2)cc1)c1coc(N2CCN(Cc3ccccc3Cl)C2=O)n1]

Following General Procedure E using ester 20 and 4-phenoxyaniline, the title product was obtained as a dark yellow oil that solidified on standing (32 mg, 61%), after purification on silica gel (5% MeOH in CH₂Cl₂).

m/z LRMS (ESI⁺): 489 (100%) [M+H]⁺. HRMS (ESI⁺): calc. for C₂₆H₂₂³⁵ClN₄O₄[M+H]⁺ 489.1330. found 489.1332.

### Abd-L13: 2-(3-benzyl-2-oxoimidazolidin-1-yl)-N-(4-phenoxybenzyl)oxazole-4-carboxamide (22) (Abd-L13)

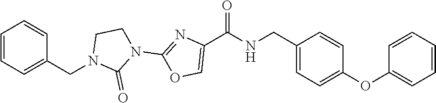 [SMILES: O=C(NCc1ccc(Oc2ccccc2)cc1)c1coc(N2CCN(Cc3ccccc3)C2=O)n1]

Following General Procedure E using ester 4 and (4-phenoxyphenyl)methanamine, the title product was obtained as a thick yellow oil that solidified on standing (17 mg, 42%), after purification on silica gel (5% MeOH in CH₂Cl₂).

m/z LRMS (ESI⁺): 469 (100%) [M+H]⁺. HRMS (ESI⁺): calc. for C₂₇H₂₅N₄O₄ [M+H]⁺ 469.1876. found 469.1874.

### Abd-L14: 2-(3-(2-chlorobenzyl)-2-oxoimidazolidin-1-yl)-N-(4-phenoxybenzyl)oxazole-4-carboxamide (23) (Abd-L14)

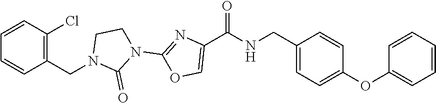 [SMILES: O=C(NCc1ccc(Oc2ccccc2)cc1)c1coc(N2CCN(Cc3ccccc3Cl)C2=O)n1]

Following General Procedure E using ester 20 and (4-phenoxyphenyl)methanamine, the title product was obtained as a dark orange oil that solidified on standing (21 mg, 53%), after purification on silica gel (5% MeOH in CH₂Cl₂).

m/z LRMS (ESI⁺): 503 (100%) [M+H]⁺. HRMS (ESI⁺): calc. for C₂₇H₂₄₃₅ClN₄O₄[M+H]⁺ 503.1486. found 503.1484.

### Abd-L15: 1-(3-methoxybenzyl)piperazine (24)

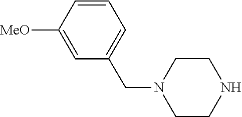 [SMILES: COc1cccc(CN2CCNCC2)c1]

Following General Procedure B using Boc-piperazine (1.00 g, 5.36 mmol, 1.1 eq.), K₂CO₃ (1.70 g, 12.2 mmol, 2.5 eq.) and 3-methoxybenzylbromide (683 μL, 4.88 mmol, 1.0 eq.) the title compound was obtained as a colourless oil that solidified on standing after purification on silica gel (10% EtOAc in pentane).

m/z LRMS (ESI⁺): 307 (100%) [M+H]⁺.

The product was dissolved in CH₂Cl₂ (5 mL) before addition of TFA (500 μL). The resulting solution was stirred for 18 h at room temperature and concentrated in vacuo. The title compound was used in the next step without further purification (987 mg, 98% over two steps).

m/z LRMS (ESI⁺): 207 (100%) [M+H]⁺.

### ethyl 2-(4-(3-methoxybenzyl)piperazin-1-yl)oxazole-4-carboxylate (25)

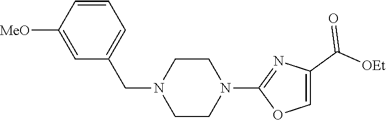 [SMILES: CCOC(=O)c1coc(N2CCN(Cc3cccc(OC)c3)CC2)n1]

Following General Procedure D using piperazine 24 (150 mg, 0.728 mmol, 1.2 eq.) and ethyl 2-chlorooxazole-4-carboxylate (116 mg, 0.661 mmol, 1.0 eq.), the title compound 25 was obtained as a yellow oil (163 mg, 71%), after purification on silica gel (4% MeOH in CH₂Cl₂). m/z LRMS (ESI⁺): 346 (100%) [M+H]⁺.

### N-(4-(1H-pyrrol-1-yl)phenyl)-2-(4-(3-methoxybenzyl)piperazin-1-yl)oxazole-4-carboxamide (26)(Abd-L15)

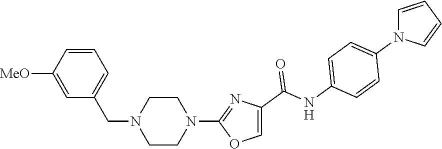 [SMILES: COc1cccc(CN2CCN(c3nc(C(=O)Nc4ccc(-n5cccc5)cc4)co3)CC2)c1]

Following General Procedure E using ester 25 and 4-pyrroleaniline, the title product was obtained as a light brown powder (52 mg, 74%), after purification on silica gel (7% MeOH in CH₂Cl₂).

m/z LRMS (ESI⁺): 458 (100%) [M+H]⁺. HRMS (ESI⁺): calc. for C₂₆H₂₈N₅O₃ [M+H]⁺ 458.2192. found 458.2192.

### N-(4-(benzyloxy)phenyl)-2-(4-(3-methoxybenzyl)piperazin-1-yl)oxazole-4-carboxamide (27) (Abd-L16)

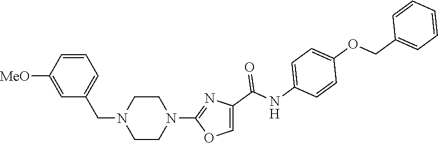 [SMILES: COc1cccc(CN2CCN(c3nc(C(=O)Nc4ccc(OCc5ccccc5)cc4)co3)CC2)c1]

Following General Procedure E using ester 26 and 4-(benzyloxy)aniline, the title product was obtained as a yellow oil that solidified on standing (37 mg, 69%), after purification on silica gel (5% MeOH in CH₂Cl₂). m/z LRMS (ESI⁺): 499 (100%) [M+H]⁺. HRMS (ESI⁺): calc. for C₂₉H₃₁N₄O₄ [M+H]⁺ 499.2345. found 499.2345.

### Abd-L17 ethyl 2-(4-(3-methoxybenzyl)piperazin-1-yl)thiazole-4-carboxylate (28)

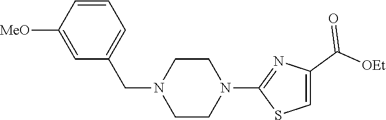 [SMILES: CCOC(=O)c1csc(N2CCN(Cc3cccc(OC)c3)CC2)n1]

Following General Procedure D using piperazine 24 (150 mg, 0.728 mmol, 1.2 eq.) and ethyl 2-chlorothiazole-4-carboxylate (126 mg, 0.661 mmol, 1.0 eq.), the title compound 28 was obtained as a yellow oil (212 mg, 89%), after purification on silica gel (4% MeOH in CH₂Cl₂). m/z LRMS (ESI⁺): 362 (100%) [M+H]⁺.

### N-(4-(benzyloxy)phenyl)-2-(4-(3-methoxybenzyl)piperazin-1-yl)thiazole-4-carboxamide (29) (Abd-L17)

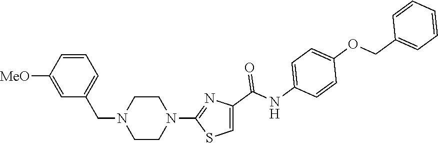 [SMILES: COc1cccc(CN2CCN(c3nc(C(=O)Nc4ccc(OCc5ccccc5)cc4)cs3)CC2)c1]

Following General Procedure E using ester 28 and 4-(benzyloxy)aniline, the title product was obtained as a yellow oil that solidified on standing (42 mg, 78%), after purification on silica gel (4% MeOH in CH₂Cl₂). m/z LRMS (ESI⁺): 515 (100%) [M+H]⁺. HRMS (ESI⁺): calc. for C₂₉H₃₁N₄O₃³²S [M+H]⁺ 515.2117. found 515.2116.

### Abd-L18: N-(4-(1H-pyrrol-1-yl)phenyl)-2-(4-(3-methoxybenzyl)piperazin-1-yl)thiazole-4-carboxamide (30) (Abd-L18)

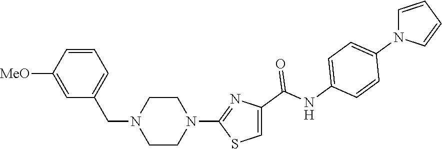 [SMILES: COc1cccc(CN2CCN(c3nc(C(=O)Nc4ccc(-n5cccc5)cc4)cs3)CC2)c1]

Following General Procedure E using ester 28 and 4-pyrroleaniline, the title product was obtained as a beige solid (37 mg, 75%), after purification on silica gel (4% MeOH in CH₂Cl₂).

m/z LRMS (ESI⁺): 474 (100%) [M+H]⁺. HRMS (ESI⁺): calc. for C₂₉H₃₁N₄O₃³²S [M+H]⁺ 474.1964. found 474.1965.

### Abd-L19: 1-(4-methoxybenzyl)piperazine (31)

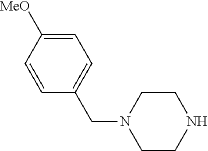 [SMILES: COc1ccc(CN2CCNCC2)cc1]

Following General Procedure B using Boc-piperazine (1.00 g, 5.36 mmol, 1.1 eq.), K₂CO₃ (1.70 g, 12.2 mmol, 2.5 eq.) and 4-methoxybenzylbromide (700 μL, 4.88 mmol, 1.0 eq.) the title compound was obtained as a colourless oil that solidified on standing after purification on silica gel (10% EtOAc in pentane). m/z LRMS (ESI⁺): 307 (100%) [M+H]⁺. The product was dissolved in CH₂Cl₂ (5 mL) before addition of TFA (500 μL). The resulting solution was stirred for 18 h at room temperature and concentrated in vacuo. The title compound was used in the next step without further purification (891 mg, 89% over two steps).

m/z LRMS (ESI⁺): 207 (100%) [M+H]⁺.

### ethyl 2-(4-(4-methoxybenzyl)piperazin-1-yl)oxazole-4-carboxylate (32)

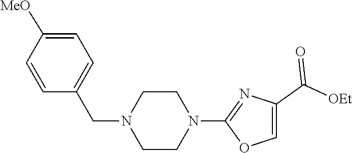 [SMILES: CCOC(=O)c1coc(N2CCN(Cc3ccc(OC)cc3)CC2)n1]

Following General Procedure D using piperazine 31 (150 mg, 0.728 mmol, 1.2 eq.) and ethyl 2-chlorooxazole-4-carboxylate (116 mg, 0.661 mmol, 1.0 eq.), the title compound 32 was obtained as a yellow oil (163 mg, 71%), after purification on silica gel (4% MeOH in CH₂Cl₂). m/z LRMS (ESI⁺): 346 (100%) [M+H]⁺.

### N-(4-(1H-pyrrol-1-yl)phenyl)-2-(4-(4-methoxybenzyl)piperazin-1-yl)oxazole-4-carboxamide (33) (Abd-L19)

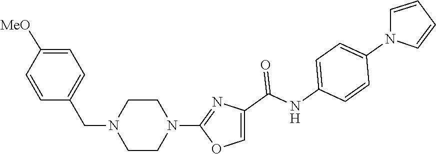 [SMILES: COc1ccc(CN2CCN(c3nc(C(=O)Nc4ccc(-n5cccc5)cc4)co3)CC2)cc1]

Following General Procedure E using ester 32 and 4-pyrroleaniline, the title product was obtained as a brown powder (24 mg, 67%), after purification on silica gel (7% MeOH in CH₂Cl₂).

m/z LRMS (ESI⁺): 458 (100%) [M+H]⁺. HRMS (ESI⁺): calc. for C₂₆H₂₈N₅O₃ [M+H]⁺ 458.2192. found 458.2192.

### Abd-L20 1-(2-methoxybenzyl)piperazine (34)

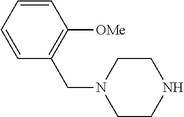 [SMILES: COc1ccccc1CN1CCNCC1]

Following General Procedure B using Boc-piperazine (200 mg, 1.07 mmol, 1.1 eq.), K₂CO₃ (370 mg, 2.68 mmol, 2.5 eq.) and 2-methoxybenzylbromide (195 mg, 0.972 mmol, 1.0 eq.) the title compound was obtained as a colourless oil that solidified on standing after purification on silica gel (10% EtOAc in pentane).

m/z LRMS (ESI⁺): 307 (100%) [M+H]⁺.

The product was dissolved in CH₂Cl₂ (5 mL) before addition of TFA (500 μL). The resulting solution was stirred for 18 h at room temperature and concentrated in vacuo. The title compound was used in the next step without further purification (164 mg, 82% over two steps).

m/z LRMS (ESI⁺): 207 (100%) [M+H]⁺.

### ethyl 2-(4-(2-methoxybenzyl)piperazin-1-yl)thiazole-4-carboxylate (35)

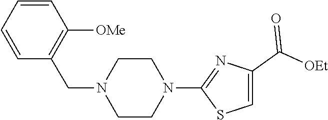 [SMILES: CCOC(=O)c1csc(N2CCN(Cc3ccccc3OC)CC2)n1]

Following General Procedure D using piperazine 34 (100 mg, 0.728 mmol, 1.2 eq.) and ethyl 2-chlorothiazole-4-carboxylate (126 mg, 0.661 mmol, 1.0 eq.), the title compound 28 was obtained as a yellow oil (216 mg, 92%), after purification on silica gel (4% MeOH in CH₂Cl₂). m/z LRMS (ESI⁺): 362 (100%) [M+H]⁺.

### N-(4-(1H-pyrrol-1-yl)phenyl)-2-(4-(2-methoxybenzyl)piperazin-1-yl)thiazole-4-carboxamide (36) (Abd-L20)

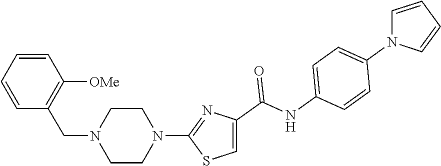 [SMILES: COc1ccccc1CN1CCN(c2nc(C(=O)Nc3ccc(-n4cccc4)cc3)cs2)CC1]

Following General Procedure E using ester 35 and 4-pyrroleaniline, the title product was obtained as a yellow oil that solidified on standing (28 mg, 72%), after purification on silica gel (6% MeOH in CH₂Cl₂).

m/z LRMS (ESI⁺): 474 (100%) [M+H]⁺. HRMS (ESI⁺): calc. for C₂₆H₂₈N₅O₃ [M+H]⁺ 474.1864. found 474.1863.

### 2-(4-(3-methoxybenzyl)piperazin-1-yl)-N-(4-(trifluoromethoxy)phenyl)thiazole-4-carboxamide (37) (Abd-L21)

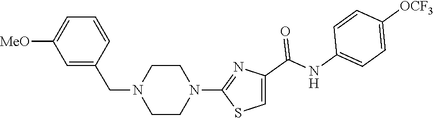 [SMILES: COc1cccc(CN2CCN(c3nc(C(=O)Nc4ccc(OC(F)(F)F)cc4)cs3)CC2)c1]

Following General Procedure E using ester 28 and 4-(trifluoromethoxy)aniline, the title product was obtained as a dark yellow oil that solidified on standing (28 mg, 76%), after purification on silica gel (4% MeOH in CH₂Cl₂).

m/z LRMS (ESI⁺): 493 (100%) [M+H]⁺. HRMS (ESI⁺): calc. for C₂₃H₂₄F₃N₄O₃³²S [M+H]⁺ 493.1521. found 493.1520.

### 2-(4-(3-methoxybenzyl)piperazin-1-yl)-N-(6-methoxypyridin-3-yl)thiazole-4-carboxamide (38) (Abd-L22)

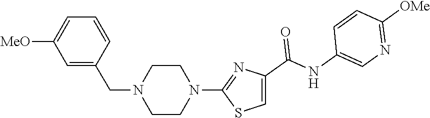 [SMILES: COc1cccc(CN2CCN(c3nc(C(=O)Nc4ccc(OC)nc4)cs3)CC2)c1]

Following General Procedure E using ester 28 and 6-methoxypyridin-3-amine, the title product was obtained as a red oil (23 mg, 71%), after purification on silica gel (4% MeOH in CH₂Cl₂).

m/z LRMS (ESI⁺): 440 (100%) [M+H]⁺. HRMS (ESI⁺): calc. for C₂₂H₂₅N₅O₃³²S [M+H]⁺ 440.1756. found 440.1754.

### 2-(4-(3-methoxybenzyl)piperazin-1-yl)-N-(2-methoxypyrimidin-5-yl)thiazole-4-carboxamide (39) (Abd-L23)

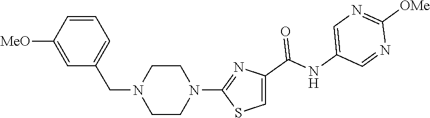 [SMILES: COc1cccc(CN2CCN(c3nc(C(=O)Nc4cnc(OC)nc4)cs3)CC2)c1]

Following General Procedure E using ester 28 and 6-methoxypyridin-3-amine, the title product was obtained as a light yellow oil (23 mg, 71%), after purification on silica gel (4% MeOH in CH₂Cl₂).

m/z LRMS (ESI⁺): 441 (100%) [M+H]⁺. HRMS (ESI⁺): calc. for C₂₁H₂N₆O₃³²S [M+H]⁺ 441.1709. found 441.1710.

### Abd-L24: 1-(3-methoxybenzyl)tetrahydropyrimidin-2(1H)-one (40)

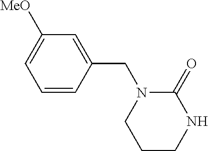 [SMILES: COc1cccc(CN2CCCNC2=O)c1]

Following General Procedure A using tetrahydro-2(1H)-pyrimidinone (500 mg, 5.00 mmol, 1.0 eq.), NaH (60% suspension in oil, 134 mg, 5.00 mmol, 1.0 eq.) and 3-methoxybenzylbromide (630 μL, 4.50 mmol, 0.9 eq.), the title compound was obtained as a colourless oil that solidified on standing (564 mg, 57%), after purification on silica gel (5% MeOH in CH₂Cl₂).

m/z LRMS (ESI⁺): 221 (100%) [M+H]⁺.

### ethyl 2-(3-(3-methoxybenzyl)-2-oxotetrahydropyrimidin-1(2H)-yl)oxazole-4-carboxylate (41)

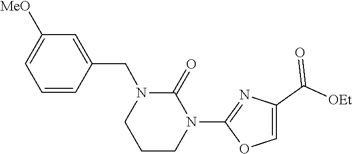 [SMILES: CCOC(=O)c1coc(N2CCCN(Cc3cccc(OC)c3)C2=O)n1]

Following General Procedure C using cyclic urea 40 (132 mg, 0.375 mmol, 1.1 eq.) and ethyl 2-chlorooxazole-4-carboxylate (60 mg, 0.341 mmol, 1.0 eq.), the title compound 41 was obtained as a yellow oil (51 mg, 42%), after purification on silica gel (3% MeOH in CH₂Cl₂). m/z LRMS (ESI⁺): 360 (100%) [M+H]⁺.

### N-(4-(1H-pyrrol-1-yl)phenyl)-2-(3-(3-methoxybenzyl)-2-oxotetrahydropyrimidin-1(2H)-yl)oxazole-4-carboxamide (42) (Abd-L24)

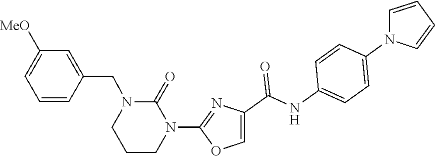 [SMILES: COc1cccc(CN2CCCN(c3nc(C(=O)Nc4ccc(-n5cccc5)cc4)co3)C2=O)c1]

Following General Procedure E using ester 41 and 4-pyrroleaniline, the title product was obtained as a dark yellow oil (12 mg, 41%), after two purifications on silica gel (5% MeOH in CH₂Cl₂).

m/z LRMS (ESI⁺): 472 (100%) [M+H]⁺. HRMS (ESI⁺): calc. for C₂₆H₂₆N₅O₄[M+H]⁺ 472.1985. found 472.1986.

### Abd-L25: ethyl 2-(4-(3-methoxybenzyl)piperazin-1-yl)pyrimidine-4-carboxylate (43)

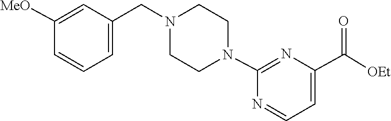 [SMILES: CCOC(=O)c1ccnc(N2CCN(Cc3cccc(OC)c3)CC2)n1]

Following General Procedure D using piperazine 24 (133 mg, 0.645 mmol, 1.2 eq.) and ethyl 2-chloropyrimidine-4-carboxylate (100 mg, 0.538 mmol, 1.0 eq.), the title compound 43 was obtained as a pale yellow solid (178 mg, 93%), after purification on silica gel (4% MeOH in CH₂Cl₂). m/z LRMS (ESI⁺): 357 (100%) [M+H]⁺.

### N-(4-(1H-pyrrol-1-yl)phenyl)-2-(4-(3-methoxybenzyl)piperazin-1-yl)pyrimidine-4-carboxamide (44) (Abd-L25)

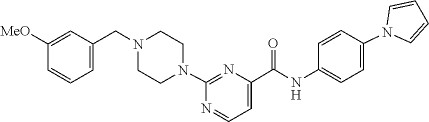 [SMILES: COc1cccc(CN2CCN(c3nccc(C(=O)Nc4ccc(-n5cccc5)cc4)n3)CC2)c1]

Following General Procedure E using ester 43 and 4-pyrroleaniline, the title product was obtained as a beige solid (35 mg, 89%), after purification on silica gel (4% MeOH in CH₂Cl₂). m/z LRMS (ESI⁺): 469 (100%) [M+H]⁺. HRMS (ESI⁺): calc. for C₂₇H₂₉N₆O₂ [M+H]⁺ 469.2352. found 469.2353.

### Abd-L26 (PAL compound): phenyl(4-(piperazin-1-ylmethyl)phenyl)methanone (45)

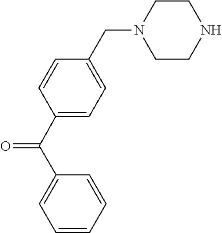 [SMILES: O=C(c1ccccc1)c1ccc(CN2CCNCC2)cc1]

Following General Procedure B using Boc-piperazine (1.00 g, 5.36 mmol, 1.1 eq.), K₂CO₃ (1.70 g, 12.2 mmol, 2.5 eq.) and 4-bromomethylbenzophenone (1.34 g, 4.88 mmol, 1.0 eq.) the title compound was obtained as a colourless oil that solidified on standing after purification on silica gel (8% EtOAc in pentane). m/z LRMS (ESI⁺): 381 (100%) [M+H]⁺. The product was dissolved in CH₂Cl₂ (10 mL) before addition of TFA (1 mL). The resulting solution was stirred for 18 h at room temperature and concentrated in vacuo. The title compound was used in the next step without further purification (1.32 g, 88% over two steps).

m/z LRMS (ESI⁺): 281 (100%) [M+H]⁺.

### Ethyl 2-(4-(4-benzoylbenzyl)piperazin-1-yl)oxazole-4-carboxylate (46)

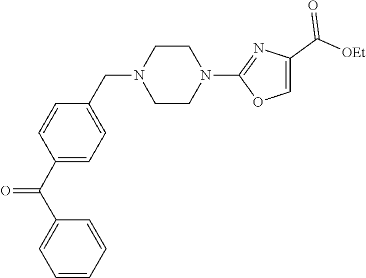 [SMILES: CCOC(=O)c1coc(N2CCN(Cc3ccc(C(=O)c4ccccc4)cc3)CC2)n1]

Following General Procedure D using piperazine 45 (580 mg, 2.07 mmol, 1.2 eq.) and ethyl 2-chlorooxazole-4-carboxylate (326 mg, 1.86 mmol, 1.0 eq.), the title compound 46 was obtained as a yellow oil (630 mg, 81%), after purification on silica gel (3% MeOH in CH₂Cl₂).

m/z LRMS (ESI⁺): 420 (100%) [M+H]⁺.

### N-(4-aminophenyl)-2-(4-(4-benzoylbenzyl)piperazin-1-yl)oxazole-4-carboxamide (47)

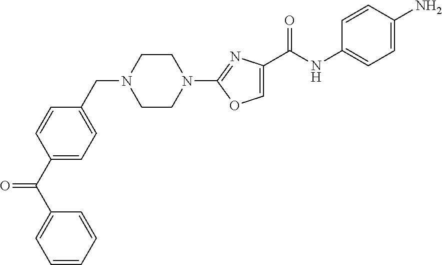 [SMILES: Nc1ccc(NC(=O)c2coc(N3CCN(Cc4ccc(C(=O)c5ccccc5)cc4)CC3)n2)cc1]

Following General Procedure E using ester 46 and tert-butyl-4-aminophenylcarbamate, the Boc protected product was obtained as a pale yellow oil that solidified on standing (82 mg, 73%), after purification on silica gel (25% EtOAc in CH₂Cl₂). m/z LRMS (ESI⁺): 582 (100%) [M+H]⁺. The product was dissolved in CH₂Cl₂ (2 mL) before addition of TFA (150 μL). The resulting solution was stirred for 18 h at room temperature and concentrated in vacuo. The title compound was used in the next step without further purification (66 mg, 99%).

m/z LRMS (ESI⁺): 482 (100%) [M+H]⁺.

### benzyl(2-(2-(2-((4-(2-(4-(4-benzoylbenzyl)piperazin-1-yl)oxazole-4-carboxamido)phenyl)amino)-2-oxoethoxy)ethoxy)ethyl)carbamate (48)

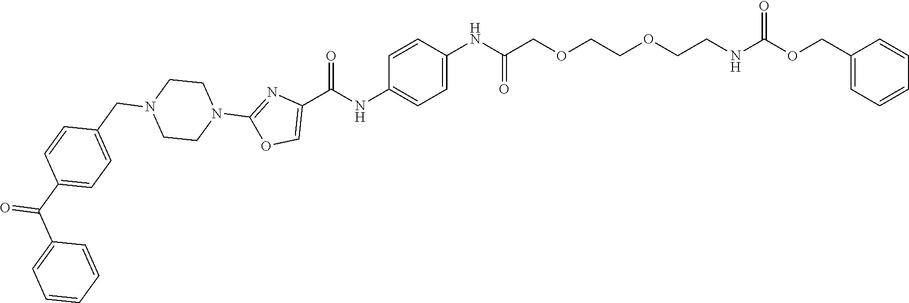 [SMILES: O=C(COCCOCCNC(=O)OCc1ccccc1)Nc1ccc(NC(=O)c2coc(N3CCN(Cc4ccc(C(=O)c5ccccc5)cc4)CC3)n2)cc1]

Aniline 47 (60 mg, 0.125 mmol, 1.0 eq.) was dissolved in DMF (2 mL) before sequential addition of N,N-diisopropylethylamine (65 μL, 0.375 mmol, 3.0 eq.), 3-oxo-1-phenyl-2,7,10-trioxa-4-azadodecan-12-oic acid (45 mg, 0.150 mmol, 1.2 eq.) and HATU (67 mg, 0.175 mmol, 1.4 eq.). The resulting solution was stirred for 18 h, diluted with EtOAc (10 mL) and washed with brine/water (1:1, 3×50 mL). The organic phase was dried (Na₂SO₄), filtered and concentrated in vacuo. The crude material was then purified on silica gel (9% MeOH in CH₂Cl₂) to afford the title compound as a yellow oil (72 mg, 76%).

m/z LRMS (ESI⁺): 761 (100%) [M+H]⁺.

### 2-(4-(4-benzoylbenzyl)piperazin-1-yl)-N-(4-(2-(2-(2-(5-((3aR,4R,6aS)-2-oxohexahydro-1H-thieno[3,4-d]imidazol-4-yl)pentanamido)ethoxy)ethoxy)acetamido)phenyl)oxazole-4-carboxamide (49) (Abd-L26)

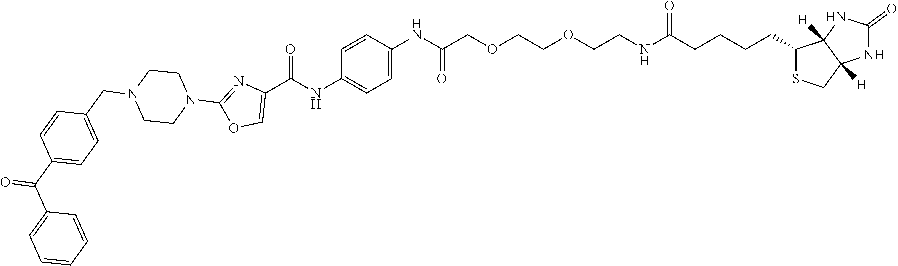 [SMILES: O=C(CCCC[C@H]1SC[C@H]2NC(=O)N[C@H]21)NCCOCCOCC(=O)Nc1ccc(NC(=O)c2coc(N3CCN(Cc4ccc(C(=O)c5ccccc5)cc4)CC3)n2)cc1]

Carbamate 48 was dissolved in THE (2 mL) before addition of MeOH (200 μL). The solution was degassed with nitrogen for 5 min before addition of a catalytic amount of Pd/C. The suspension was degassed with nitrogen for a further 5 min before replacing the atmosphere with hydrogen (by mean of a balloon). The reaction was monitored by TLC and MS; after 2 h, the reaction was complete. The balloon was removed and the reaction was passed through a bed of Celite™ using EtOAc and MeOH as an eluent. The product was used in the next step without further purification. The obtained amine (32 mg, 0.050 mmol, 1.0 eq.) was dissolved in DMF (1 mL) before sequential addition of N,N-diisopropylethylamine (26 μL, 0.150 mmol, 3.0 eq.), D-biotin (15 mg, 0.060 mmol, 1.2 eq.) and HATU (27 mg, 0.070 mmol, 1.4 eq.). The resulting solution was stirred for 18 h, diluted with EtOAc (10 mL) and washed with brine/water (1:1, 3×20 mL). The organic phase was dried (Na₂SO₄), filtered and concentrated in vacuo. The crude material was purified on silica gel (10% MeOH in CH₂Cl₂) and further via prep TLC (15% MeOH in CH₂Cl₂) to afford the title compound as a yellow oil (12 mg, 28%).

m/z LRMS (ESI⁺): 853 (100%) [M+H]⁺.

While specific embodiments of the invention have been described herein for the purpose of reference and illustration, various modifications will be apparent to a person skilled in the art without departing from the scope of the invention as defined by the appended claims.

### REFERENCES

Assi, S. A., Tanaka, T., Rabbitts, T. H., and Fernandez-Fuentes, N. (2010). PCRPi: Presaging Critical Residues in Protein interfaces, a new computational tool to chart hot spots in protein interfaces. Nucleic Acids Res 38, e86.
Bataille, C. J. R., Rabbitts, T. H., and Claridge, T. D. (2020). NMR waterLOGSY as an assay in drug development programmes for detecting protein-ligand interactions. Bio-Protocols in press.
Bavetsias, V., Lanigan, R. M., Ruda, G. F., Atrash, B., McLaughlin, M. G., Tumber, A., Mok, N. Y., Le Bihan, Y. V., Dempster, S., Boxall, K. J., et al. (2016). 8-Substituted Pyrido[3,4-d]pyrimidin-4(3H)-one Derivatives As Potent, Cell Permeable, KDM4 (JMJD2) and KDM5 (JARID1) Histone Lysine Demethylase Inhibitors. J Med Chem 59, 1388-1409.
Bery, N., Cruz-Migoni, A., Bataille, C. J., Quevedo, C. E., Tulmin, H., Miller, A., Russell, A., Phillips, S. E., Carr, S. B., and Rabbitts, T. H. (2018). BRET-based RAS biosensors that show a novel small molecule is an inhibitor of RAS-effector protein-protein interactions. Elife 7.
Bery, N., Legg, S., Debreczeni, J., Breed, J., Embrey, K., Stubbs, C., Kolasinska-Zwierz, P., Barrett, N., Marwood, R., Watson, J., et al. (2019). KRAS-specific inhibition using a DARPin binding to a site in the allosteric lobe. Nat Commun 10, 2607.
Bery, N., and Rabbitts, T. H. (2019). Bioluminescence Resonance Energy Transfer 2 (BRET2)-Based RAS Biosensors to Characterize RAS Inhibitors. Curr Protoc Cell Biol, e83.
Boehm, T., Foroni, L., Kaneko, Y., Perutz, M. F., and Rabbitts, T. H. (1991). The rhombotin family of cysteine-rich LIM-domain oncogenes: distinct members are involved in T-cell translocations to human chromosomes 11p15 and 11p13. Proc Natl Acad Sci USA 88, 4367-4371.
Chambers, J., and Rabbitts, T. H. (2015). LMO2 at 25 years: a paradigm of chromosomal translocation proteins. Open Biol 5, 150062.
Cochet, O., Kenigsberg, M., Delumeau, I., Virone-Oddos, A., Multon, M. C., Fridman, W. H., Schweighoffer, F., Teillaud, J. L., and Tocque, B. (1998). Intracellular expression of an antibody fragment-neutralizing p21 ras promotes tumor regression. Cancer Res 58, 1170-1176.
Cruz-Migoni, A., Canning, P., Quevedo, C. E., Bataille, C. J. R., Bery, N., Miller, A., Russell, A. J., Phillips, S. E. V., Carr, S. B., and Rabbitts, T. H. (2019). Structure-based development of new RAS-effector inhibitors from a combination of active and inactive RAS-binding compounds. Proc Natl Acad Sci USA 116, 2545-2550.
Dalvit, C., Fogliatto, G., Stewart, A., Veronesi, M., and Stockman, B. (2001). WaterLOGSY as a method for primary NMR screening: practical aspects and range of applicability. J Biomol NMR 21, 349-359.
Ferrando, A. A., and Look, A. T. (2003). Gene expression profiling in T-cell acute lymphoblastic leukemia. Semin Hematol 40, 274-280.
Gupta, A., Xu, J., Lee, S., Tsai, S. T., Zhou, B., Kurosawa, K., Werner, M. S., Koide, A., Ruthenburg, A. J., Dou, Y., et al. (2018). Facile target validation in an animal model with intracellularly expressed monobodies. Nat Chem Biol 14, 895-900.
Lavoie, H., Thevakumaran, N., Gavory, G., Li, J. J., Padeganeh, A., Guiral, S., Duchaine, J., Mao, D. Y., Bouvier, M., Sicheri, F., et al. (2013). Inhibitors that stabilize a closed RAF kinase domain conformation induce dimerization. Nat Chem Biol 9, 428-436.
McCormack, M. P., Forster, A., Drynan, L., Pannell, R., and Rabbitts, T. H. (2003). The LMO2 T-cell oncogene is activated via chromosomal translocations or retroviral insertion during gene therapy but has no mandatory role in normal T-cell development. Mol Cell Biol 23, 9003-9013.
Nam, C. H., Lobato, M. N., Appert, A., Drynan, L. F., Tanaka, T., and Rabbitts, T. H. (2008). An antibody inhibitor of the LMO2-protein complex blocks its normal and tumorigenic functions. Oncogene 27, 4962-4968.
Quevedo, C. E., Cruz-Migoni, A., Bery, N., Miller, A., Tanaka, T., Petch, D., Bataille, C. J. R., Lee, L. Y. W., Fallon, P. S., Tulmin, H., et al. (2018). Small molecule inhibitors of RAS-effector protein interactions derived using an intracellular antibody fragment. Nat Commun 9, 3169.
Rabbitts, T. H. (2009). Commonality but diversity in cancer gene fusions. Cell 137, 391-395.
Royer-Pokora, B., Loos, U., and Ludwig, W. D. (1991). TTG-2, a new gene encoding a cysteine-rich protein with the LIM motif, is overexpressed in acute T-cell leukaemia with the t(11;14)(p13;q11). Oncogene 6, 1887-1893.
Ryan, D. P., Sunde, M., Kwan, A. H., Marianayagam, N. J., Nancarrow, A. L., Vanden Hoven, R. N., Thompson, L. S., Baca, M., Mackay, J. P., Visvader, J. E., et al. (2006). Identification of the key LMO2-binding determinants on Ldb1. J Mol Biol 359, 66-75.
Sadakane, Y., and Hatanaka, Y. (2006). Photochemical fishing approaches for identifying target proteins and elucidating the structure of a ligand-binding region using carbene-generating photoreactive probes. Anal Sci 22, 209-218.
Scott, D. E., Bayly, A. R., Abell, C., and Skidmore, J. (2016). Small molecules, big targets: drug discovery faces the protein-protein interaction challenge. Nat Rev Drug Discov 15, 533-550.
Sewell, H., Tanaka, T., El Omari, K., Mancini, E. J., Cruz, A., Fernandez-Fuentes, N., Chambers, J., and Rabbitts, T. H. (2014). Conformational flexibility of the oncogenic protein LMO2 primes the formation of the multi-protein transcription complex. Sci Rep 4, 3643.
Smith, E., and Collins, I. (2015). Photoaffinity labeling in target- and binding-site identification. Future Med Chem 7, 159-183.
Spencer-Smith, R., Koide, A., Zhou, Y., Eguchi, R. R., Sha, F., Gajwani, P., Santana, D., Gupta, A., Jacobs, M., Herrero-Garcia, E., et al. (2017). Inhibition of RAS function through targeting an allosteric regulatory site. Nat Chem Biol 13, 62-68.
Tanaka, T., and Rabbitts, T. H. (2003). Intrabodies based on intracellular capture frameworks that bind the RAS protein with high affinity and impair oncogenic transformation. EMBO J 22, 1025-1035.
Tanaka, T., and Rabbitts, T. H. (2008). Interfering with protein-protein interactions: potential for cancer therapy. Cell Cycle 7, 1569-1574.
Tanaka, T., and Rabbitts, T. H. (2010). Interfering with RAS-effector protein interactions prevent RAS-dependent tumour initiation and causes stop-start control of cancer growth. Oncogene 29, 6064-6070.
Tanaka, T., Sewell, H., Waters, S., Phillips, S. E., and Rabbitts, T. H. (2011). Single domain intracellular antibodies from diverse libraries: emphasizing dual functions of LMO2 protein interactions using a single VH domain. J Biol Chem 286, 3707-3716.
Tanaka, T., Williams, R. L., and Rabbitts, T. H. (2007). Tumour prevention by a single antibody domain targeting the interaction of signal transduction proteins with RAS. EMBO J 26, 3250-3259.
Visintin, M., Tse, E., Axelson, H., Rabbitts, T. H., and Cattaneo, A. (1999). Selection of antibodies for intracellular function using a two-hybrid in vivo system. Proc Natl Acad Sci USA 96, 11723-11728.
Wadman, I., Li, J., Bash, R. O., Forster, A., Osada, H., Rabbitts, T. H., and Baer, R. (1994). Specific in vivo association between the bHLH and LIM proteins implicated in human T cell leukemia. EMBO J 13, 4831-4839.
Wadman, I. A., Osada, H., Grutz, G. G., Agulnick, A. D., Westphal, H., Forster, A., and Rabbitts, T. H. (1997). The LIM-only protein Lmo2 is a bridging molecule assembling an erythroid, DNA-binding complex which includes the TAL1, E47, GATA-1 and Ldb1/NLI proteins. EMBO J 16, 3145-3157.

1. A compound, or a pharmaceutically acceptable salt, hydrate or solvate thereof, having the structural Formula (I) shown below: 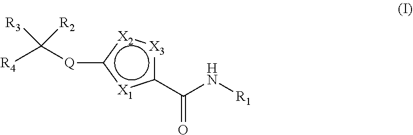
wherein:
R₁ is selected from:
(i) (1-6C)alkyl which is optionally substituted by one or more Rₐ;
(ii) a group of the formula: 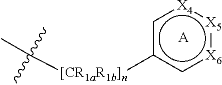
wherein [symbol]
denotes the point of attachment;
n is 0 or 1;
R₁ₐ and R_{1b} are selected from hydrogen or methyl;
X₄, X₅ and X₆ are selected from C—H, C—Rₐ or N;
(iii) a group of the formula: 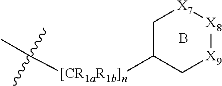
wherein [symbol]
denotes the point of attachment;
n, R₁ₐ and R_{1b} are as defined above;
Ring B is a saturated or partially unsaturated ring;
X₇, X₈ and X₉ are selected from C—H, C—Rₐ or N if a bond connecting them to an adjacent atom is an unsaturated double bond, or C—H₂, C—HRₐ, C—(Rₐ)₂, N—H, N—R_{b}, S or O if the bonds attaching them to adjacent atoms are single bonds;
wherein each Rₐ is independently selected from (1-4C)alkyl, halo, (1-4C)haloalkyl, (1-4C)haloalkoxy, cyano, nitro, (3-6C)cycloalkyl, (3-6C)cycloalkyl(1-2C)alkyl, phenyl, (CH₂)_{q1}NR_{ab}R_{ac}, (CH₂)_{q1}OR_{ab}, (CH₂)_{q1}C(O)R_{ab}, (CH₂)_{q1}C(O)OR_{ab}, (CH₂)_{q1}OC(O)R_{ab}, (CH₂)_{q1}C(O)N(R_{ac})R_{ab}, (CH₂)_{q1}N(R_{ac})C(O)R_{ab}, (CH₂)_{q1}S(O)ₚR_{ab} (where p is 0, 1 or 2), (CH₂)_{q1}SO₂N(R_{ac})R_{1ab}, or (CH₂)_{q1}N(R_{ac})SO₂R_{ab},
and wherein:
q1 is 0, 1, 2 or 3;
R_{ab} is selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl and heterocyclyl(1-2C)alkyl,
and wherein R_{ab} is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, nitro, NR_{ad}Rₐₑ, OR_{ad}, C(O)R_{ad}, C(O)OR_{ad}, OC(O)R_{ad}, C(O)N(Rₐₑ)R_{ad}, N(Rₐₑ)C(O)R_{ad}, S(O)ₚR_{ad} (where p is 0, 1 or 2), SO₂N(Rₐₑ)R_{ad}, N(Rₐₑ)SO₂R_{ad}, or (CH₂)_{q2}NR_{ad}Rₐₑ (where q2 is 1, 2 or 3); wherein R_{ad} and Rₐₑ are each independently selected from hydrogen or (1-6C)alkyl;
and R_{ac} is selected from hydrogen or (1-2C)alkyl;
or where R_{ab} and R_{ac} are linked to a common N atom, they may be linked such that, together with the N atom to which they are attached, they form a 5 or 6 membered heteroaryl ring or a 5 to 7 membered heterocyclic ring, each of which is optionally substituted as for R_{ab} above;
wherein R_{b} is independently selected from (1-4C)alkyl, (1-4C)haloalkyl or —C(O)R_{ba}, wherein R_{ba} is selected from (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl and heterocyclyl(1-2C)alkyl, and wherein R_{ba} is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, nitro, NR_{bd}R_{be}, OR_{bd}, C(O)R_{bd}, C(O)OR_{bd}, OC(O)R_{bd}, C(O)N(R_{be})R_{bd}, N(R_{be})C(O)R_{bd}, S(O)ₚR_{bd} (where p is 0, 1 or 2), SO₂N(R_{be})R_{bd}, N(R_{be})SO₂R_{bd}, or (CH₂)_{q3}NR_{bd}R_{be} (where q3 is 1, 2 or 3); wherein R_{bd} and R_{b}e are each independently selected from hydrogen or (1-6C)alkyl;
X₁, X₂ and X₃ are selected from N, N—R₅, O, S and CR₆, wherein R₅ is hydrogen or methyl and R₆ is hydrogen, methyl or halo, with the proviso that at least one of X₁, X₂ and X₃ is selected from N, N—R₅, O and S;
Q is a group of the formula: 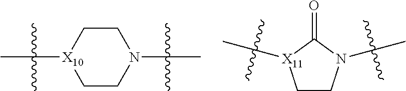
or —X₁₂—CH₂—CH₂—NR₇— or —X₁₃—C(O)—NR₈—;
wherein:
X₁₀ and X₁₁ are selected from N or CH;
X₁₂ and X₁₃ are selected from NR₉, CH₂, CHR₉ or C(R₉)₂; and
R₇, R₈ and R₉ are selected from hydrogen or (1-2C)alkyl;
R₂ and R₃ are selected from hydrogen or (1-2C)alkyl;
R₄ is a phenyl, heteroaryl, or heterocyclyl ring optionally substituted by (1-4C)alkyl, halo, (1-4C)haloalkyl, (1-4C)haloalkoxy, (1-4C)aminoalkyl, (1-4C)hydroxyalkyl, cyano, nitro, NR₄ₐR_{4b}, OR₄ₐ, C(O)R₄ₐ, C(O)OR₄ₐ, OC(O)R₄ₐ, C(O)N(R_{4b})R₄ₐ, N(R_{4b})C(O)R₄ₐ, S(O)ₚR₄ₐ (where p is 0, 1 or 2), SO₂N(R_{4b})R₄ₐ, N(R_{4b})SO₂R₄ₐ, or (CH₂)_{q4}NR₄ₐR_{4b} (where q4 is 1, 2 or 3); wherein R₄ₐ is selected from hydrogen, (1-4C)alkyl, (3-6C)cycloalkyl, (3-6C)cycloalkyl(1-2C)alkyl, phenyl aryl(1-2C)alkyl, heteroaryl, heteroaryl(1-2C)alkyl, heterocyclyl and heterocyclyl(1-2C)alkyl, and wherein:
R₄ₐ is optionally further substituted by (1-4C)alkyl, halo, (1-4C)haloalkyl, (1-4C)haloalkoxy, cyano, nitro, NR₄ₐₐR_{4ab}, OR₄ₐₐ, C(O)R₄ₐₐ, C(O)OR₄ₐₐ, OC(O)R₄ₐₐ, C(O)N(R_{4ab})R₄ₐₐ, N(R_{4ab})C(O)R₄ₐₐ, S(O)ₚR₄ₐₐ (where p is 0, 1 or 2), SO₂N(R_{4ab})R₄ₐₐ, N(R_{4ab})SO₂R₄ₐₐ, or (CH₂)_{q5}NR₄ₐₐR_{4ab} (where q5 is 1, 2 or 3) and R₄ₐₐ and R_{4ab} are hydrogen or (1-2C)alkyl;
R_{4b} is selected from hydrogen or (1-2C)alkyl;
or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above.

2. A compound according to claim 1, or a pharmaceutically acceptable salt, hydrate or solvate thereof, wherein R₁ is selected from:
(i) (1-4C)alkyl which is optionally substituted by one or more Rₐ;
(ii) a group of the formula: 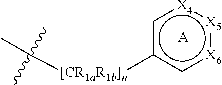
wherein [symbol]
denotes the point of attachment;
n is 0 or 1;
R₁ₐ and R_{1b} are selected from hydrogen;
X₄, X₅ and X₆ are selected from C—H, C—Rₐ or N;
(iii) a group of the formula: 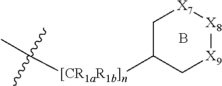
wherein [symbol]
denotes the point of attachment;
n, R₁ₐ and R_{1b} are as defined above;
Ring B is a saturated ring;
X₇, X₈ and X₉ are selected from C—H;
wherein each Rₐ is independently selected from methyl, halo, (CH₂)_{q1}NR_{ab}R_{ac}, (CH₂)_{q1}OR_{ab}, (CH₂)_{q1}C(O)R_{ab} or (CH₂)_{q1}C(O)OR_{ab},
and wherein:
q1 is 0;
R_{ab} is selected from hydrogen, (1-4C)alkyl, aryl, aryl(1-2C)alkyl,
and wherein R_{ab} is optionally further substituted by one or more substituent groups independently selected from, halo;
and R_{ac} is selected from hydrogen;
or where R_{ab} and R_{ac} are linked to a common N atom, they may be linked such that, together with the N atom to which they are attached, they form a 5 membered heteroaryl ring or a 5 or 6 membered heterocyclic ring, each of which is optionally substituted as for R_{ab} above.

3. A compound according to any one of the preceding claims, or a pharmaceutically acceptable salt, hydrate or solvate thereof, wherein R₁ is selected from:
(i) a group of the formula: 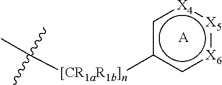
wherein [symbol]
denotes the point of attachment;
n is 0 or 1;
R₁ₐ and R_{1b} are selected from hydrogen;
X₄, X₅ and X₆ are selected from C—H, C—Rₐ or N;
(ii) a group of the formula: 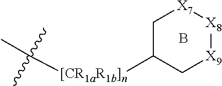
wherein [symbol]
denotes the point of attachment;
n is 0,
R₁ₐ and R_{1b} are selected from hydrogen
Ring B is a saturated ring;
X₇, X₈ and X₉ are selected from C—H;
wherein each Rₐ is independently selected from methyl, halo, (CH₂)_{q1}NR_{ab}R_{ac}, (CH₂)_{q1}OR_{ab}, (CH₂)_{q1}C(O)R_{ab} or (CH₂)_{q1}C(O)OR_{ab},
and wherein:
q1 is 0;
R_{ab} is selected from hydrogen, (1-2C)alkyl, phenyl, benzyl,
and wherein R_{ab} is optionally further substituted by one or more substituent groups independently selected from, halo;
and R_{ac} is selected from hydrogen;
or where R_{ab} and R_{ac} are linked to a common N atom, they may be linked such that, together with the N atom to which they are attached, they form a 5 membered heteroaryl ring or a 5 or 6 membered heterocyclic ring, each of which is optionally substituted as for R_{ab} above.

4. A compound according to any one of the preceding claims, or a pharmaceutically acceptable salt, hydrate or solvate thereof, wherein R₁ is selected from: 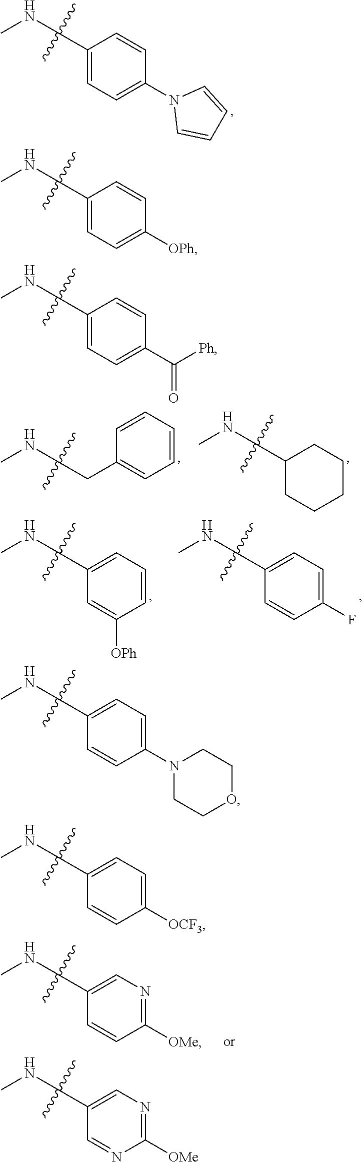
wherein [symbol]
denotes the point of attachment.

5. A compound according to any one of the preceding claims, or a pharmaceutically acceptable salt, hydrate or solvate thereof, wherein X₁, X₂ and X₃ are selected from N, O, S and CR₆, wherein R₆ is hydrogen, methyl or halo, with the proviso that at least one of X₁, X₂ and X₃ is selected from N, O and S.

6. A compound according to any one of the preceding claims, or a pharmaceutically acceptable salt, hydrate or solvate thereof, wherein X₁ is N; X₂ is O or S; and X₃ is CR₆, wherein R₆ is hydrogen.

7. A compound according to any one of the preceding claims, or a pharmaceutically acceptable salt, hydrate or solvate thereof, wherein Q is a group of the formula: 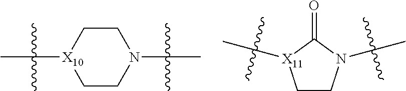
or —X₁₂—CH₂—CH₂—NR₇— or —X₁₃—C(O)—NR₈—;
wherein:
X₁₀ and X₁₁ are selected from N;
X₁₂ and X₁₃ are selected from NRs; and
R₇, R₈ and R₉ are selected from hydrogen.

8. A compound according to any one of the preceding claims, or a pharmaceutically acceptable salt, hydrate or solvate thereof, wherein Q is selected from 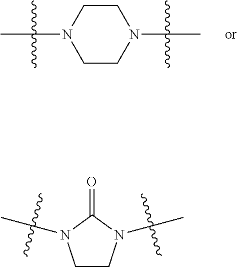
wherein [symbol] denotes the point of attachment.

9. A compound according to any one of the preceding claims, or a pharmaceutically acceptable salt, hydrate or solvate thereof, wherein R₂ and R₃ are selected from hydrogen or methyl.

10. A compound according to any one of the preceding claims, or a pharmaceutically acceptable salt, hydrate or solvate thereof, wherein R₂ and R₃ are hydrogen.

11. A compound according to any one of the preceding claims, or a pharmaceutically acceptable salt, hydrate or solvate thereof, wherein R₄ is a phenyl, heteroaryl, or heterocyclyl ring optionally substituted by (1-2C)alkyl, halo, (1-2C)haloalkyl, (1-2C)haloalkoxy, (1-2C)aminoalkyl, (1-2C)hydroxyalkyl, cyano, nitro, NR₄ₐR_{4b}, OR₄ₐ, C(O)R₄ₐ, C(O)OR₄ₐ, OC(O)R₄ₐ, C(O)N(R_{4b})R₄ₐ, N(R_{4b})C(O)R₄ₐ, S(O)ₚR₄ₐ (where p is 0, 1 or 2), SO₂N(R_{4b})R₄ₐ, N(R_{4b})SO₂R₄ₐ, or (CH₂)_{q4}NR₄ₐR_{4b} (where q4 is 1, 2 or 3); wherein R₄ₐ is selected from hydrogen, (1-2C)alkyl, phenyl aryl(1-2C)alkyl, heteroaryl, heteroaryl(1-2C)alkyl, heterocyclyl and heterocyclyl(1-2C)alkyl, and wherein:
R₄ₐ is optionally further substituted by (1-2C)alkyl, halo, (1-2C)haloalkyl, (1-2C)haloalkoxy, cyano, nitro;
R_{4b} is selected from hydrogen or (1-2C)alkyl;
or R₄ₐ and R_{4b} are linked to a common N atom, they may be linked such that, together with the N atom to which they are attached, they form a 5 or 6 membered heteroaryl ring or a 5 to 7 membered heterocyclic ring, each of which is optionally substituted as for R₄ above.

12. A compound according to any one of the preceding claims, or a pharmaceutically acceptable salt, hydrate or solvate thereof, wherein R₄ is a phenyl ring optionally substituted by halo, cyano, nitro, NR₄ₐR_{4b}, OR₄ₐ, C(O)R₄ₐ, N(R_{4b})C(O)R₄ₐ; wherein R₄ₐ is selected from hydrogen, (1-2C)alkyl, phenyl aryl(1-2C)alkyl; and R_{4b} is selected from hydrogen.

13. A compound according to any one of the preceding claims, or a pharmaceutically acceptable salt, hydrate or solvate thereof, wherein R₄ is selected from 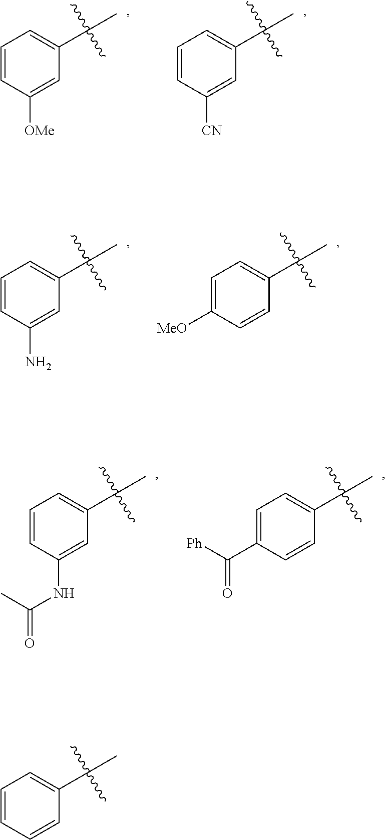
wherein [symbol]
denotes the point of attachment.

14. A compound of the formula: 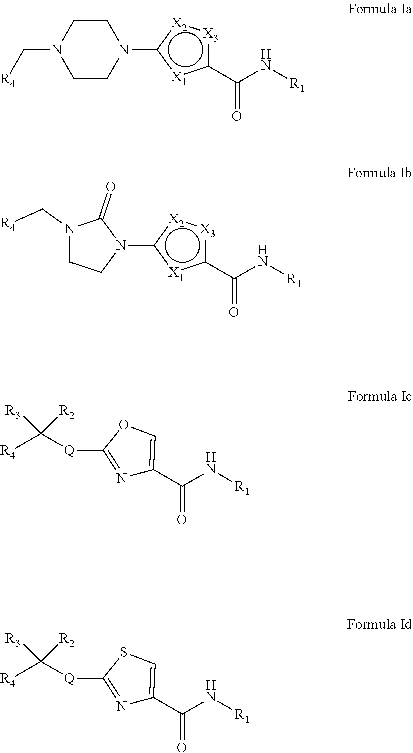
wherein R₁, X₁, X₂, X₃, Q, R₂, R₃ and R₄ are each as defined in any one of claims 1 to 13.

15. A compound, or a pharmaceutically acceptable salt, hydrate or solvate thereof, selected from any one of the following:
2-(3-benzyl-2-oxoimidazolidin-1-yl)-N-(4-phenoxyphenyl)oxazole-4-carboxamide (Abd-L6);
N-(4-(1H-pyrrol-1-yl)phenyl)-2-(3-(3-chlorobenzyl)-2-oxoimidazolidin-1-yl)oxazole-4-carboxamide (Abd-L7);
2-(3-(4-chlorobenzyl)-2-oxoimidazolidin-1-yl)-N-(3,4-dimethoxyphenyl)oxazole-5-carboxamide (Abd-L8);
N-(4-(benzyloxy)phenyl)-2-(3-(3-methoxybenzyl)-2-oxoimidazolidin-1-yl)oxazole-4-carboxamide (Abd-L9);
N-(4-(1H-pyrrol-1-yl)phenyl)-2-(3-(3-cyanobenzyl)-2-oxoimidazolidin-1-yl)oxazole-4-carboxamide (Abd-L10);
2-(3-(2-chlorobenzyl)-2-oxoimidazolidin-1-yl)-N-(4-phenoxyphenyl)oxazole-4-carboxamide (Abd-L12);
2-(3-benzyl-2-oxoimidazolidin-1-yl)-N-(4-phenoxybenzyl)oxazole-4-carboxamide (Abd-L13);
2-(3-(2-chlorobenzyl)-2-oxoimidazolidin-1-yl)-N-(4-phenoxybenzyl)oxazole-4-carboxamide (Abd-L14);
N-(4-(1H-pyrrol-1-yl)phenyl)-2-(4-(3-methoxybenzyl)piperazin-1-yl)oxazole-4-carboxamide (Abd-L15);
N-(4-(benzyloxy)phenyl)-2-(4-(3-methoxybenzyl)piperazin-1-yl)oxazole-4-carboxamide (Abd-L16);
N-(4-(benzyloxy)phenyl)-2-(4-(3-methoxybenzyl)piperazin-1-yl)thiazole-4-carboxamide (Abd-L17);
N-(4-(1H-pyrrol-1-yl)phenyl)-2-(4-(3-methoxybenzyl)piperazin-1-yl)thiazole-4-carboxamide (Abd-L18);
N-(4-(1H-pyrrol-1-yl)phenyl)-2-(4-(4-methoxybenzyl)piperazin-1-yl)oxazole-4-carboxamide (Abd-L19);
N-(4-(1H-pyrrol-1-yl)phenyl)-2-(4-(2-methoxybenzyl)piperazin-1-yl)thiazole-4-carboxamide (Abd-L20);
2-(4-(3-methoxybenzyl)piperazin-1-yl)-N-(4-(trifluoromethoxy)phenyl)thiazole-4-carboxamide (Abd-L21);
2-(4-(3-methoxybenzyl)piperazin-1-yl)-N-(6-methoxypyridin-3-yl)thiazole-4-carboxamide (Abd-L22); and
2-(4-(3-methoxybenzyl)piperazin-1-yl)-N-(2-methoxypyrimidin-5-yl)thiazole-4-carboxamide (Abd-L23).

16. A pharmaceutical composition comprising a compound according to any one claims 1 to 15, or a pharmaceutically acceptable salt, hydrate or solvate thereof, in admixture with a pharmaceutically acceptable diluent or carrier.

17. A compound according to any one of claims 1 to 15, or a pharmaceutically acceptable salt or hydrate thereof, for use in therapy.

18. A compound according to any one of claims 1 to 15, or a pharmaceutically acceptable salt, hydrate or solvate thereof, or a pharmaceutical composition according to claim 16, for use in a method of inhibiting cell proliferation, such as the treatment of cancer.

19. The compound or pharmaceutical composition according to claim 18, wherein said cancer is a haematological cancer.

20. A method of treating a proliferative disorder in a patient in need of such treatment, the method comprising administering a therapeutically effective amount of a compound according to any one of claims 1 to 15, or a pharmaceutically acceptable salt, hydrate or solvate thereof, or a pharmaceutical composition according to claim 16.

21. A method of treating cancer in a patient in need of such treatment, the method comprising administering a therapeutically effective amount of a compound according to any one of claims 1 to 15, or a pharmaceutically acceptable salt, hydrate or solvate thereof, or a pharmaceutical composition according to claim 16.

22. The method according to claim 21, wherein said cancer is a haematological cancer.
